(19) United States
(12) Patent Application Publication
(10) Pub. No.: US 2023/0085555 A1
(43) Pub. Date: Mar. 16, 2023

(54) A NON-INVASIVE CONTINUOUS BLOOD GLUCOSE MONITOR

(71) Applicant: OPUZ PTY LTD, St. Peters, New South Wales (AU)

(72) Inventors: Mobin Nomvar, St. Peters, New South Wales (AU); Shane Cox, St. Peters, New South Wales (AU); Thomas Telfer, St. Peters, New South Wales (AU); David Wang, St. Peters, New South Wales (AU)

(21) Appl. No.: 17/795,451

(22) PCT Filed: Jan. 29, 2021
(86) PCT No.: PCT/AU2021/050057

§ 371 Date: Jul. 26, 2022

(30) Foreign Application Priority Data
Jan. 29, 2020 (AU) 2020900228

(51) Int. Cl. A61B 5/145; A61B 5/00; A61B 5/0537

(52) U.S. Cl. CPC A61B 5/14532; A61B 5/7264; A61B 5/6843; A61B 5/0537; A61B 2562/0209; A61B 2562/04

(57) ABSTRACT

Provided herein is a non-invasive device for measuring glucose levels (i.e., concentration) in a subject, preferably a human subject. The present invention relates to a wearable device, a kit and a method thereof for measuring blood glucose concentrations/levels. The non-invasive devices of the present invention can be used as wearable devices such as a smart band, ring, bracelet, watch and the like to monitor the blood glucose levels in diabetics without discomfort and stress due to finger pricks by measuring bio-impedance data.

This application claims priority from Australian Provisional Patent Application No. 2020900228 filed 29 Jan. 2020, the contents of which should be understood to be incorporated.

### FIELD OF THE INVENTION

The present invention relates to a non-invasive device for measuring glucose levels (i.e., concentration) in a subject, preferably a human subject.

In particular, the present disclosure relates to a wearable device, a kit and a method thereof for measuring blood glucose concentrations/levels. These non-invasive devices can be used as wearable devices, such as a smart band or watch, to monitor the blood glucose levels in diabetics without discomfort and stress due to finger pricks by measuring bio-impedance data. However, it will be appreciated that the invention is not limited to this particular field of use.

### BACKGROUND OF THE INVENTION

The following discussion of the prior art is provided to place the invention in an appropriate technical context and enable the advantages of it to be more fully understood. It should be appreciated, however, that any discussion of the prior art throughout the specification should not be considered as an express or implied admission that such prior art is widely known or forms part of the common general knowledge in the field.

In the past 30 years, the rates of obesity have grown significantly as a result of more processed foods and higher levels of sugars in beverages. Global obesity rates among adults have increased by over 25% and even more dramatic increases have been observed among children and young adults by almost 50%. To date, the number of overweight and obese people worldwide have increased from about 800 million in 1980 to over 2 billion people in 2013. Currently, there are over 600 million obese people globally.

Studies have shown that obesity can increase the likelihood of developing diabetes (diabetes mellitus). Diabetes is a chronic disease characterised by high levels of glucose in the blood. Blood sugar levels are controlled by insulin, a hormone produced by the pancreas. Diabetes occurs when the pancreas is (i) unable to produce enough insulin, (ii) the body becomes resistant to insulin, or (iii) both. The two common forms of diabetes are:
Type 1 diabetes: an auto-immune disease where the body's immune system attacks the insulin producing cells of the pancreas. Type 1 diabetes is a result of the pancreas's failure to produce enough insulin due to loss of beta cells. People with type 1 diabetes cannot produce insulin and require lifelong insulin injections for survival; and
Type 2 diabetes: a condition in which cells fail to respond to insulin appropriately and typically begins with insulin resistance. In some cases or as the disease progresses, a lack of insulin may occur. Type 2 diabetes is typically related to hereditary factors and lifestyle risk factors including poor diet, insufficient physical activity and being overweight or obese.

Diabetics who need treatment try to maintain blood glucose levels within a specified range prescribed by a health professional. Currently, the only reliable way to self-measure blood glucose levels is to use a conventional blood glucose monitor. However, conventional blood glucose monitors are invasive, inconvenient, painful and can cause discomfort. To monitor blood glucose concentration, a user pricks their finger with a lancet and a droplet of blood is added onto a blood glucose checking strip. This strip is then inserted into the meter, which reads the strip and displays the blood glucose concentration.

Non-invasive approaches have been developed for measuring blood glucose concentrations in a subject. These approaches typically measure impedance of skin tissue. However, commercial applications of bio-impedance to measure blood glucose levels have been limited.

PCT/US1998/002037 discloses a method and apparatus for non-invasively determining glucose level in fluid of subject, typically blood glucose level. Impedance of skin tissue is measured and the measurement is used with impedance measurements previously correlated with directly determined glucose levels to determine the glucose level from the newly measured impedance.

PCT/RU2013/000144 discloses a method for measuring the impedance of a human body skin tissue region at a high frequency and a low frequency with the aid of electrodes fastened to the human body and measuring the blood glucose concentration by determining the value of the volume of extracellular fluid.

However, these methods and devices discussed above have been limited to measuring bio-impedance on the skin tissue. As such, the reproducibility, repeatability and accuracy of the blood glucose measurements of bio-impedance based devices have been poor which is a critical drawback; and incorrect measurement of glucose concentration in blood can have serious health consequences to diabetics.

It is an object of the present invention to overcome or ameliorate one or more of the disadvantages of the prior art, or at least to provide a useful alternative.

It is an object of at least one preferred form of the present invention to provide a method or device which can accurately measure blood glucose concentration in a subject.

Although the invention will be described with reference to specific examples it will be appreciated by those skilled in the art that the invention may be embodied in many other forms.

### SUMMARY OF THE INVENTION

Bio-electrical impedance (bio-impedance) measurements have been used to measure physiological parameters in biological applications to characterise cells. These measurements include measuring body composition (such as body fat and muscle mass), total body water and other applications. Bioimpedance measurements have also been used for disease diagnostic applications.

However, there has been limited application of bioimpedance measurements to measure blood glucose concentration. In fact, the Applicant believes there are currently no commercial products on the market that use impedance to measure blood glucose concentration in a subject.

The development of non-invasive blood glucose devices using impedance measurements has been challenging as the measurements typically have poor quality or weak signals resulting in poor reproducibility, repeatability and accuracy of the blood glucose concentration measurements. Therefore, the development of a suitable non-invasive blood glucose device using impedance for commercialisation has remained a significant challenge. This is because electrodes in these devices have been limited to measuring impedance only on the skin tissue which results in poor quality or weak signals.

According to one aspect, the present invention provides a non-invasive device for determining blood glucose concentration in a subject, the device comprising:
at least two electrodes for contacting the subject's skin and adapted to be connected to a receiver for measuring an impedance signal; and
a housing adapted to receive the electrodes;
   wherein the electrodes are configured such that an electrical current passes through a portion of a subject in use.

According to another aspect, the present invention provides a non-invasive device for determining blood glucose concentration in a subject, the device comprising:
at least two electrodes for contacting the subject's skin and adapted to be connected to a receiver for measuring an impedance signal;
a housing adapted to receive the electrodes; and
a probe for measuring at least one additional physiological parameter.

The present Applicant has surprisingly found that by placing electrodes in a configuration which provides electrical current to pass through a portion of the body rather than just on the surface of the skin provides a device which can measure high quality signals for reproducible, repeatable and accurate measurement of blood glucose concentration.

Without being bound by any one theory, the present Applicant has surprisingly found that the electrical current can pass through a portion of the body (for example a finger) through at least one of dermis layers, fat layers, muscles, bone and the like. The electrical current can pass through different portions of the body, for example, portions of the electrical current can pass through the dermis layers, fat layers, body fluid and combinations thereof. Further, electrical currents of different frequencies will have different path combinations. In some embodiments, the non-invasive device comprises three, four, five, six, seven, eight, nine or ten electrodes. In preferred embodiments, the non-invasive device comprises four electrodes.

In certain embodiments, when the non-invasive device comprises two or three electrodes, there can be in some embodiments the possibility that the positive and negative current injecting and voltage measurement electrodes short circuit. However, in a four-electrode non-invasive device, the risk of short circuiting can be reduced or prevented as the current injecting and voltage measurement electrodes are independent (separated). In this preferred embodiment, since there is no current flow passing through the voltage measurement electrodes, there is no loading effect on the electrodes that would otherwise be caused by the current injecting electrodes.

As would be appreciated by a skilled addressee, a non-invasive device comprising two or three electrodes can be used in the present invention because it can still function equivalently to a preferred four electrode system, however, since biological systems are more complex (such as for measuring blood glucose level), there is a possibility that a two or three electrode device can under- or overestimate measurement values because an electrode can be both a current injecting and/or voltage measurement electrode. Four electrodes can prevent any electrical issues (short-circuiting) and provide greater sensitivity because each electrode can be independently a separate current injecting electrode (i− and i+) and voltage measurement electrode (v− and v+). As such, a higher sensitivity and reducing or preventing a short circuit can be provided using a preferred embodiment device of four electrodes.

For example, if the device comprises two or three electrodes, there is no even distribution of the i−, i+, v− and v+ electrodes/probes. In an embodiment comprising two or three electrodes, four contact points (i−, i+, v− and v+) will have to be distributed across the number of contact points/electrodes which can potentially increase the probability of the device short circuiting because the probes can be on the same terminal.

An intrinsic property of bioimpedance is sensitivity. A higher level of sensitivity can be achieved with a four-electrode device. The present inventors surprisingly found that four electrodes have improved effectiveness for measuring bioimpedance from a narrow specific range in biological systems compared to a two or three electrode device.

In some embodiments, a single electrode can inject current to the skin of the subject and measure voltage. In other embodiments, the electrodes independently inject current and measure voltage in separate circuits. In preferred embodiments, the non-invasive device comprises a stimulating electrode and a sensing electrode. That is, one electrode will inject current and another electrode will measure the voltage response. For example, for a non-invasive device comprising four electrodes, two electrodes can inject current while two electrodes can measure the voltage.

It was surprisingly found that when making impedance measurements, the benefit of separating the current injecting (stimulating) electrode from the voltage measurement (sensing) electrodes was that any loading or polarisation of the current injecting electrodes would not affect the voltage measurement performance. In these embodiments, there should be no current flowing in to or out of the voltage sensing paths as only the voltage (or potential) response of the subject due to the stimulating current should be sensed.

In some embodiments, the non-invasive device comprises at least two component devices such that each component device comprises at least one electrode. In these embodiments, one component device comprising at least one electrode can be a stimulating device and another component device comprising at least one electrode can be a sensing device.

As would be appreciated, the electrodes can be positioned in any suitable configuration provided such that an electrical current passes through a portion of a subject. In some embodiments, the electrodes are substantially distributed evenly over the portion of the subject. In some embodiments, the electrodes are substantially opposed to each other. In some embodiments, the electrodes are configured to be radially spaced between about greater than about 20° to less than about 180°, greater than about 30° to less than about 180°, between about 40° to less than about 180°, about 50° to less than about 180°, about 70° to less than about 180°, about 90° to less than about 180°, about 120° to less than about 180°, about 150° to less than about 180°. In some embodiments, the electrodes are configured to be spaced less than about 180°, less than about 150°, less than about 120°, less than about 90°, less than about 45°, less than about 30° about a point of reference. In some embodiments, a current injecting (stimulating) electrode is substantially opposed to a voltage measurement (sensing) electrode. In some embodiments, a positive electrode is substantially opposed to a negative electrode.

In preferred embodiments, the device comprises four electrodes. In this embodiment, two electrodes are substantially opposed to each other along an axis. For example, when the device is in the form a ring, the two electrodes are positioned about 180° from each other. In this embodiment, each of the additional electrodes (i.e., the additional two electrodes) are configured to be radially spaced between about greater than about 5° to less than about 80°, between about greater than about 5° to less than about 60°, between about greater than about 5° to less than about 50°, between about greater than about 20° to less than about 40°, preferably about 30° or about 60° relative to each of the electrodes. In preferred embodiments, each of the additional electrodes (i.e., the additional two electrodes) are configured to be radially spaced between about greater than about 5° to less than about 80°, between about greater than about 5° to less than about 60°, between about greater than about 5° to less than about 50°, between about greater than about 20° to less than about 40°, preferably about 30° relative to each of the electrodes and the additional electrodes are substantially opposed to each other. As would be appreciated by a skilled addressee, the term “substantially opposed” means that the centre of mass of the electrode and/or additional electrodes are configured to be about 180° to each other, however, the contact angle of the electrode surface can be any suitable angle.

In certain embodiments, the two electrodes are current injecting electrodes and the two additional electrodes are voltage measurement electrodes. In other embodiments, the two electrodes are voltage measurement electrodes and the two additional electrodes are current injecting electrodes. In certain embodiments, at least one of the electrodes is a current injecting electrode and at least one of the additional electrodes is a voltage measurement electrode.

The present inventors have found that a four-electrode non-invasive device is preferable to measure bioimpedance. Prior to the present application, it was generally believed that use of a four-electrode device to measure bioimpedance was prone to errors compared to one or two electrode configurations.

The present inventors also found that using four electrodes can avoid common mode voltage and as such reduce or prevent the electrode polarisation effect which would be experienced in a two-electrode system. Two electrode systems are the most common systems typically used for bioimpedance measurements.

In some embodiments, a voltage measurement electrode of the invention can be spaced to provide a gap between about 0.2 mm to about 1 cm, between about 0.2 mm to about 10 mm, between about 0.2 mm to about 3 mm, between about 0.2 mm to about 2 cm, between about 0.5 mm to about 1.5 mm, preferably about 1 mm relative to a current injecting electrode.

It should be appreciated by the skilled addressee that the electrode or electrodes can take any geometry or size depending on optimising the impedance signal. The electrode may take any suitable shape and may be for example in the shape of a circle, square, triangle, rhomboid, trapezoid, rectangle, pentagon, hexagon, octagon or an irregular shape. In preferred embodiments, the electrode is substantially square shaped, preferably square shaped. The present inventors surprisingly found that substantially square shaped electrodes reduced the impedance at the skin-electrode interface and were more sensitive to changes in bioimpedance than circular electrodes of similar cross-sectional surface area.

It should be appreciated that the electrode can be made from any suitable conductive material. In some embodiments, the electrode is made from a metal or salt thereof, a metal alloy, or conductive polymer. In some embodiments, the electrode is made from a material selected from the group consisting of an electroceramic, copper, aluminium, platinum, titanium, gold, silver, iron, steel, stainless steel, brass, bronze, nickel, silver/silver chloride, conductive rubber, conductive carbon such as graphite, graphene and reduced graphene oxide, and combinations thereof. In preferred embodiments, the electrode is a gold electrode or a silver/silver chloride electrode. In some embodiments, the electrode is a patch.

In some embodiments, the electrode can comprise a coating of another conductive material. In these embodiments, use of a cheaper electrode such as aluminium, stainless steel and copper can be used in combination with a coating of a desired conductive material such as gold to improve conductive contact between the skin of the subject and the electrode. The present Applicant surprisingly found that use of gold or gold coated electrode improved the signal quality of impedance measurements.

In some embodiments, the coating is made from a metal or salt thereof, a metal alloy, or conductive polymer. In some embodiments, the coating is made from a material selected from the group consisting of an electroceramic, copper, aluminium, platinum, titanium, gold, silver, iron, steel, stainless steel, brass, bronze, nickel, silver/silver chloride, conductive rubber, conductive carbon, and combinations thereof. In preferred embodiments, the coating is a gold coating.

In certain embodiments, the gold or gold-plated electrode is at least about 99%, at least about 99.5%, at least about 99.9%, at least about 99.99% or at least about 99.999% gold. In preferred embodiments, the gold or gold-plated electrode is at least about 99.99% gold.

In certain embodiments, the coating of the electrode is gold or gold-plated and is at least about 99%, at least about 99.5%, at least about 99.9%, at least about 99.99%, or at least about 99.999% gold. In preferred embodiments, the coating of the electrode is at least about 99.99% gold.

The present inventors have surprisingly found that use of a gold or gold-plated electrode can provide the least impedance at the skin-electrode interface for monitoring biometric information of a user, such as, blood glucose levels.

The coating can be applied using any suitable technique such as sputtering, electroplating, dip coating, spray coating, spin coating, adhesion and combinations thereof.

It should be appreciated by the skilled addressee that the coating of the electrode can be any suitable thickness to provide sufficiently conductive contact. In certain embodiments, the coating has a thickness of about 10 nm to 500 micron, about 100 nm to 500 micron, about 300 nm to 500 micron, about 10 to 500 micron, about 50 to 500 micron, about 100 to 500 micron, about 200 to 500 micron. In certain embodiments, the coating has a thickness less than about 500 micron, 400 micron, 300 micron, 200 micron, or 100 micron. In some embodiments, the coating has a thickness of about 0.5 mm to about 5 mm, about 0.5 mm to about 3 mm, about 0.5 mm to about 2 mm, preferably about 1 mm.

It should be appreciated by the skilled addressee that the electrode can be any suitable size depending on the size of the non-invasive device. The size of each electrode can depend on at least two factors: (i) from a physics standpoint, the electrode-skin contact area should be as large as permissible for higher quality impedance signals; and (ii) from a device and comfort standpoint, the electrode should be as small as possible.

In some embodiments, the surface area of an electrode is between about 2 to 100 mm², between about 5 to 80 mm², between about 2 to 60 mm², between about 2 to 50 mm², between about 2 to 40 mm², between about 5 to 40 mm², between about 10 to 40 mm², between about 15 to 40 mm², between about 20 to 40 mm² and preferably between about 19mm² and 36mm², more preferably about 25 mm². In preferred embodiments, each electrode has substantially about the same surface area. In certain embodiments, the surface area of an electrode is greater than about 15 mm², greater than about 20 mm², preferably greater than about 25 mm², greater than about 50 mm² and greater than about 64 mm².

These surface area of the electrode should be chosen such that they are large enough to produce a signal, but small enough as to be sufficiently spaced apart for a range of non-invasive device sizes. IEC 60601 provides international technical standards for the safety and performance of medical electrical equipment and limits current for DC and AC frequencies less than 1 kHz to 10 μA, and for AC currents above 1 kHz as per equation 1. This standard specifies the limits of patient leakage currents and patient auxiliary currents under normal conditions and single fault conditions. These current limits are important parameters in the circuit design of an electrical medical device.

$$Maximum AC current for frequencies above 1 kHz . \qquad Equation 1.$$

$${I}_{{AC}_{MAX}} = \frac{{F}_{E}}{1000 Hz} \cdot 10 u {A}_{RMS}$$

wherein I_{AC}_{_{MAX}} is the maximum AC current, 10 μA_{RMS} is 10 μA (root mean square value) and F_{E} is the excitation frequency.

For comfort of a subject when using the non-invasive device, the electrode should in certain embodiments have no surface or textural inconsistencies which can be tactually felt on a surface by a finger. This can prevent or ameliorate skin sensitisations which may occur during use.

It should be appreciated by the skilled addressee that the housing can take any geometry or size depending on the size of the electrode and the ultimate configuration of the non-invasive device. The housing may take any suitable shape and may be for example in the shape of a cube, cylinder, prism, tetrahedron or an irregular shape. In preferred embodiments, the housing is adapted to minimise electrical interference to improve signal quality such as physical and/or electrical isolation. For example, when the device is physically connected to a receiver, the housing can be adapted such that the electrical leads are positioned away from the electrodes.

In some embodiments, the housing is made from a material selected from the group consisting of a ceramic, stone, leather, silicone rubber, rubber, copper, aluminium, platinum, titanium, gold, silver, iron, steel, stainless steel, brass, bronze, nickel, wood, bone, polymer, and combinations thereof. In certain embodiments, the polymer is selected from the group consisting of polyvinyl chloride (PVC), high-density polyethylene (HDPE), high Impact Polystyrene (HIPS), polyurethane (PU), acrylonitrile butadiene styrene (ABS), polyhydroxyalkanoates (PHA), polyhydroxybutyrate (PHB), polyvinylalcohol-polycaprolactone (PVOH-PCL), polyglycolic acid (PGA), polycaprolactone (PCL), polylactic acid (PLA), polyethylene (PE), polystyrene (PS), polypropylene (PP) and combinations thereof.

It should be appreciated by the skilled addressee that any suitable ceramic can be used. Suitable ceramics can be selected from the group consisting of an inorganic or non-metallic (such as oxide, nitride or carbide) material. Suitable ceramic materials can be selected from the group consisting of a earthenware (such as porcelain and clay), barium titanate, bismuth strontium calcium copper oxide, boron oxide, boron nitride, ferrite, lead zirconate titanate, magnesium diboride, silicon aluminium oxynitride, silicon carbide, silicon nitride, steatite, titanium carbide, yttrium barium copper oxide, zinc oxide, zirconium dioxide, partially stabilised zirconia, calcium sulfate, hydroxyapatite, Bioglass®, calcium silicate, Bioverit®, Ceraverit® and combinations thereof. It should be appreciated by the skilled addressee that any suitable stone can be used. In certain embodiments, the stone is a gemstone or the like used for jewelry. In certain embodiments, the stone is selected from the group consisting of amber, amethyst, emerald, jade, jasper, onyx, diamond, quartz, ruby, sapphire, turquoise, cubic zirconia and combinations thereof.

It should be appreciated by the skilled addressee that any suitable wood can be used. In certain embodiments, the wood is a hardwood or softwood. In some embodiments, the wood is a heartwood or sapwood. In some embodiments, the wood is selected from the group consisting of bamboo, timber, pine, teak, spruce, larch, juniper, aspen, hornbeam, birch, alder, beech, oak, elm, cherry, pear, maple, linden, ash, cedar, fir, mahogany, walnut and combinations thereof.

It should be appreciated by the skilled addressee that any suitable bone can be used. Typical bones are those which are used for decorative purposes. In certain embodiments, the bones can be obtained from cow, sheep, fish, whale, seal, dolphin, bird, deer, ox, moose, kangaroo, alligator, rabbit, guinea pig and combinations thereof.

In embodiments wherein the housing is made of polymer, the housing can further comprise an additive. The addition of additives to the housing can be used to tailor the physical and chemical properties of the resulting materials formed therefrom.

In one embodiment, the additives can be selected from the group consisting of an antioxidant, a thermostabiliser, a plasticiser, a filler, a surfactant, a lubricant, a pigment, a tackifier, a stabiliser and combinations thereof.

The antioxidant can be of any suitable compound to prevent or minimise oxidative degradation reactions of the housing including phenols and phosphites. In one embodiment, the antioxidant is selected from the group consisting of pentaerythritol tetrakis, octadecyl-3-(3,5-di-tert-butyl-4-hydroxyphenyl)-propionate, benzenepropanoic acid, 3,5-bis(1,1-dimrhtyl-ethyl)-4-hydroxy-C7-C9 branched alkyl esters, 3′,3′,3′,5,5′,5′-hexa-tert-butyl-a,a′,a′-(mesitylene-2,4,6-trityl)tri-p-cresol, tris-(3,5-di-tert-butyl-4-hydroxybenzyl) isocyanurate, 2′,3-bis[3-(3,5-di-tert-butyl-4-hydroxyphenyl)propionyl]-propionohydrazide, N,N′-hexane-1,6-diylbis(3-(3,5-di-tert-butyl-4-hydroxyphenyl-propionamide)), 4,6-bis(dodecylthiomethyl)-o-cresol, 4,6-bis(octylthiomethyl)-o-cresol, 2,2′-methylenebis(4-methyl-6-tert-butylphenol), 2,6-di-tert-butyl-4-[[4,6-bis(octylthio)-1,3,5-triazin-2-yl]amino]phenol, tris(2,4-di-tert-butylphenyl)-phosphite, bis(2,4-di-tert-butylphenyl)pentaerythritol diphosphate and combinations thereof.

The thermostabiliser can be of any suitable compound to improve the resistance of the housing to discoloration. The thermostabiliser can be a lead compound, organotin compound, other metal compound and organic stabiliser. In one embodiment, the thermostabiliser is selected from the group consisting of lead sulphite, lead carbonate, lead stearate, dibutyl tin maleate, barium-cadmium stearate, barium-cadmium-zinc stearate, methyl tin mercaptide, methyl tin ester, butyl tin thioglycolate, n-octyl tin mercaptide, butyl tin mercaptide, butyl tin carboxylate, 3-(2,4-dichlorophenylazo)-9-(2,3-epoxypropane)carbazole, barbituric acid, thiobarbituric acid, poly(hexamethyleneadipate), poly(ethyleneadipate), poly(hexamethylene-terephthalate) and poly(ethyleneterephthalate) and combinations thereof.

Plasticisers can be added to the housing to improve the processing characteristics, while also providing flexibility in the end-use product. Plasticisers can be selected from the group consisting of ester plasticisers, sebacates, adipates, terephthalates, dibenzoates, gluterates, phthalates, azelates and combinations thereof.

The filler can be of any suitable compound to decrease the amount of polymer required in the housing. In one embodiment, the filler is selected from the group consisting of aluminium silicate, potassium silicate, calcium silicate, silica, sodium silicate, clays, kaolin clay, aluminium oxide, limestone, barium sulfate, strontium sulfate/selestite, magnesium oxide, calcium carbonate, dolomite, metal power or flakes, ceramic beads, magnesium silicate and combinations thereof.

The surfactant can be of any suitable compound to provide a surface active film. In one embodiment, the surfactant is anionic, cationic, zwitterionic or non-ionic. In one embodiment, the surfactant comprises a functional group selected from the group consisting of sulfate, sulfonate, phosphate, carboxylate, amine, ammonium, alcohol, ether and combination thereof. In one embodiment, the surfactant is selected from the group consisting of sodium stearate, 4-(5-dodecyl) benzenesulfonate, 3-[(3-cholamidopropyl)dimethylammonio]-1-propanesulfonate, phosphatidylserine, phosphatidylethanolamine, phosphatidylcholine, octaethylene glycol monododecyl ether, pentaethylene glycol monododecyl ether, decyl glucoside, lauryl glucoside, octyl glucoside, triton X-100, nonoxynol-9, glyceryl laurate, polysorbate, dodecyldimethylamine oxide, polysorbate, cocamide monoethanolamine, cocamide diethanolamine, poloxamer, polyethoxylated tallow amine and combinations thereof.

The lubricant can be of any suitable compound to reduce the internal and/or external friction of the housing during processing. In one embodiment, the lubricant is an acid amide, acid ester, fatty acid, hydrocarbon wax, metallic soap or combination thereof. In one embodiment, the lubricant is selected from the group consisting of zinc laurate, zinc stearate, calcium laurate, calcium stearate, lead stearate, magnesium stearate, aluminium stearate, sodium stearate, tin stearate, barium stearate, cobalt stearate, paraffin wax, mineral oil, erucamide, oleamide, stearamide, ethylene bis stearamide, ethylene bis-oleamide, montan wax, stearyl stearate, distearyl pthalate, lauric acid, myristic acid, palmitic acid, stearic acid, oleic acid, erucic acid, molybdenum disulphide, mica, niobium(IV) selenide, graphite intercalated with bromine or a metal chloride, and combinations thereof.

The pigment can be of any suitable compound to impart colour to the resulting housing. In one embodiment, the pigment is an inorganic pigment or an organic pigment. In one embodiment, the pigments are derived from compounds selected from the group consisting of an acridine, anthraquinone, diarylmethane, triarylmethane, azo, diazonium, nitro, nitroso, phthalocyanine, quinone, thiazine, oxazone, oxazin, indophenol, thiazole, safranin, xanthene, fluorene, fluorone and combinations thereof. In one embodiment, the pigment is selected from the group consisting of cadmium yellow, cadmium red, cadmium green, cadmium orange, cadmium sulfoselenide, chrome yellow, chrome green, cobalt violet, cobalt blue, cerulean blue, aureolin, azurite, han purple, han blue, egyptian blue, malachite, paris green, phthalocyanine blue BN, phthalocyanine green G, verdigris, viridian, sanguine, caput mortuum, oxide red, red ochre, venetian red, prussian blue, lead white, cremnitz white, naples yellow, red lead, manganese violet, vermilion, titanium yellow, titanium beige, titanium white, titanium black, zinc white, zinc ferrite, carbon black, ivory black, yellow ochre, raw sienna, burnt sienna, raw umber, burnt umber, ultramarine, ultramarine green shade, alizarin, alizarin crimson, gamboge, cochineal red, rose madder, indigo, indian yellow, tyrian purple, quinacridone, magenta, phthalo green, phthalo blue, pigment red 170, diarylide yellow and combinations thereof.

The tackifier can be of any suitable compound to impart adhesiveness to the resulting housing. In one embodiment, the tackifier is selected from the group consisting of a rosin resin, hydrocarbon resin, terpene resin and combinations thereof. In one embodiment, the rosin resin is selected from the group consisting of rosin ester, hydrogenated rosin resin, dimerised rosin resin and combinations thereof. In one embodiment, the rosin resin is derived from wood rosin, gum rosin, tall oil rosin or combination thereof.

In one embodiment, the hydrocarbon resin is a C₅ alkyl resin, C₅ alkenyl resin, C₉ aryl resin or combination thereof. In one embodiment, the terpene resin is a terpene phenol resin, alkyl terpene resin, alkenyl terpene resin, aryl terpene resin or combination thereof.

The stabiliser can be of any suitable compound which can directly or indirectly reduce the impact of UV radiation. In one embodiment, the stabiliser is a UV absorber, hindered amine light stabiliser and combination thereof. In one embodiment, the UV absorber is a hindered phenol. In one embodiment, the stabiliser is selected from the group consisting of 4-allyloxy-2-hydroxybenzophenone, 1-aza-3,7-dioxabicyclo[3.3.0]octane-5-methanol, tris(nonylphenyl) phosphite, 1,3,5-tris(2-hydroxyethyl)isocyanurate, tris(2,4-di-tert-butylphenyl) phosphite, tris(4-tert-butyl-3-hydroxy-2,6-dimethylbenzyl) isocyanurate, 1,3,5-trimethyl-2,4,6-tris(3,5-di-tert-butyl-4-hydroxybenzyl)-benzene, triisodecyl phosphite, tetrachloro-1,4-benzoquinone, sodium D-isoascorbate monohydrate, poly[[6-[(1,1,3,3-tetramethylbutyl)amino]-s-triazine-2,4-diyl]-[(2,2,6,6-tetramethyl-4-piperidyl)imino]-hexamethylene-[(2,2,6,6-tetra-methyl-4-piperidyl)imino], 2-phenyl-5-benzimidazolesulfonic acid, pentaerythritol tetrakis(3,5-di-tert-butyl-4-hydroxyhydrocinnamate), octadecyl 3-(3,5-di-tert-butyl-4-hydroxyphenyl)propionate, 4-nitrophenol sodium salt, methylhydroquinone, 5,5′-methylenebis(2-hydroxy-4-methoxybenzophenone), 2,2′-methylenebis(6-tert-butyl-4-methylphenol), 2,2′-methylenebis(6-tert-butyl-4-ethylphenol), 2,2′-methylenebis[6-(2H-benzotriazol-2-yl)-4-(1,1,3,3-tetramethylbutyl)phenol], methyl-p-benzoquinone, 2-methoxyhydroquinone, menthyl anthranilate, 2-hydroxy-4-(octyloxy)benzophenone, 2,2′-ethylidene-bis(4,6-di-tert-butylphenol), 2-ethylhexyl salicylate, 2-ethylhexyl trans-4-methoxycinnamate, 2-ethylhexyl 2-cyano-3,3-diphenylacrylate, ethyl 2-cyano-3,3-diphenylacrylate, 5-ethyl-1-aza-3,7-dioxabicyclo[3.3.0]octane, ditridecyl 3,3′-thiodipropionate, 2-(4,6-diphenyl-1,3,5-triazin-2-yl)-5-[(hexyl)oxy]-phenol, 4,4-dimethyloxazolidine, 2,3-dimethylhydroquinone, 2′,4′-dihydroxy-3′-propylacetophenone, 2,2′-dihydroxy-4-methoxybenzophenone, 2,2′-dihydroxy-4,4′-dimethoxybenzophenone, 2,4-dihydroxybenzophenone, didodecyl 3,3′-thiodipropionate, 3′,5′-dichloro-2′-hydroxyacetophenone, 2,6-di-tert-butyl-4-(dimethylaminomethyl)phenol, 2,4-di-tert-butyl-6-(5-chloro-2H-benzotriazol-2-yl)phenol, 5-chloro-2-hydroxy-4-methyl benzophenone, 5-chloro-2-hydroxybenzophenone, 2-tert-butyl-4-ethylphenol, 2-tert-butyl-6-(5-chloro-2H-benzotriazol-2-yl)-4-methylphenol, bis(2,2,6,6-tetramethyl-4-piperidyl) sebacate, bis(1-octyloxy-2,2,6,6-tetramethyl-4-piperidyl) sebacate, 3,9-bis(octadecyloxy)-2,4,8,10-tetraoxa-3,9-diphosphaspiro[5.5]undecane, bis(octadecyl)-hydroxylamine, 3,9-bis(2,4-dicumylphenoxy)-2,4, 8, 10-tetraoxa-3,9-diphosphaspiro[5.5]undecane, 2-(4-benzoyl-3-hydroxyphenoxy)ethyl acrylate, 2-(2H-benzotriazol-2-yl)-4-(1,1,3,3-tetramethylbutyl)phenol, 2-(2H-Benzotriazol-2-yl)-4-methyl-6-(2-propenyl)phenol, 2-[3-(2H-benzotriazol-2-yl)-4-hydroxyphenyl]-ethyl methacrylate, 2-(2H-benzotriazol-2-yl)-6-dodecyl-4-methylphenol, 2-(2H-benzotriazol-2-yl)-4,6-di-tert-pentylphenol, 2-(2H-benzotriazol-2-yl)-4,6-bis(1-methyl-1-phenylethyl)phenol and combination thereof.

It should be understood that the additives as discussed above can be added to the housing in any suitable amount to provide the desired properties. In one embodiment, the additive is added to the housing in an amount of from about 0.01 to about 50 wt %, in an amount of from about 1 to about 50 wt %, in an amount of from about 10 to about 40 wt %, in an amount of from about 10 to about 30 wt % or in an amount of from about 20 to about 30 wt %.

The housing can be made using any suitable technique. For example, the housing can be made by injection moulding, carving, extrusion, blowing, rotational moulding, thermoforming, calendering, stamping, CNC machining, embossing, 3D printing, casting and extrusion.

In certain embodiments, wherein the electrode and housing are both conductive materials, the non-invasive device comprises an insulator disposed between the electrode and housing to prevent or ameliorate the risk of a short circuit or electrical interference.

As would be appreciated by a skilled addressee, it is preferable that the electrodes of the non-invasive device should substantially be in contact with the surface of the skin of a subject under constant pressure during use to minimise artefacts and poor data measurements. In certain embodiments, the non-invasive device comprises an adjustable electrode contact mechanism to ensure measurement of high-quality impedance signals while maintaining comfort to the subject. In this embodiment, the contact area of the electrode can be automatically adjusted to ensure sufficient contact between the electrode and skin of the subject to receive high quality impedance signals. For example, the adjustable electrode contact mechanism can be a screw and/or spring fastener. This can ensure that the electrodes protrude from the housing to improve contact between the electrode and skin of the subject to receive high quality impedance signals. In other embodiments, device such as a ring can be made a of resilient material and optionally comprise a break or webbing to accommodate different sizes of a portion of a body such as a finger. For example, the device such as a ring can in some embodiments accommodate expansion over a knuckle and then contraction at the base of the finger to ensure sufficient contact.

In certain embodiments, the function of the electrode can be adjusted without physical modification using a printed circuit board (PCB) which is connected to the non-invasive device such that the electrodes can be controlled by the PCB to function as a stimulating electrode, a sensing electrode or a sink. In these embodiments, adjustment of the function of the electrodes on-the-fly by the PCB can ensure measurement of high-quality impedance signals.

It should be appreciated by the skilled addressee that the non-invasive device can be in any suitable form such as a wearable device. In some embodiments, the non-invasive device can be a smart watch, belt, band (such as a waist or arm band), bracelet, ring, clip (such as for the ear or finger) or benchtop device. For example, if the non-invasive device is in the form of a waist band, the electrode can be provided as a patch which is inserted into the waist band for use on a subject. If the non-invasive device is a smart watch or ring, the electrodes can be fitted into the housing such that the device can be connected to a mobile electronic device (such as a mobile/cell phone, tablet, laptop, personal computer and the like).

In certain embodiments, the non-invasive device comprises a notification indicator. The notification indicator can be in the form of a light (such as an LED), a screen, a visual alarm, a tactile alarm, an audio alarm and combinations thereof. The indicator can show for example the operating status of the non-invasive device such as if the device is powered on/off, normal operating status, error status and the like. In some embodiments, the notification indicator can alert a subject or remote user if the blood glucose concentration is outside a predetermined range such as above or below a normal threshold range. In certain embodiments where the indicator is a screen, the indicator can provide information such as duration of operation, real-time blood glucose concentration, impedance signal strength and quality, connection status and the like.

In some embodiments, the receiver is an electrochemical impedance spectroscopy (EIS) device, a microprocessor or a microcontroller to receive the impedance signal from the electrode of the non-invasive device. In certain embodiments, non-invasive device comprises a receiver (i.e., the receiver is integral to the device). In certain embodiments, the receiver is external to the non-invasive device. In these embodiments, the receiver can be connected to the non-invasive device using a wired connection or a wireless connection to transmit the impedance data.

In certain embodiments, the non-invasive device comprises a Faraday shield to reduce interference and improve impedance signal quality.

In some embodiments, the non-invasive device comprises a probe to measure an additional physiological parameter (i.e., biometric) of a subject. For example, the probe can be used to measure body fat, muscle mass, body composition, body temperature, skin pH, skin temperature, blood pressure, heart rate and the like. In these embodiments, the probe can be an electrode, a thermocouple or a spectrophotometer. For example, if measuring heart rate, an LED source can be provided and the light signal can be measured using an LED sensor with the difference in signals compared using an algorithm to output a subject's heart rate.

In some embodiments, the non-invasive device can be formed integrally with, attached to, or at least partially surround or encompass a third-party device. Any suitable third-party device can be used which can contact the skin of a subject such that an impedance signal can be measured. For example, the third-party device can be a phone; a phone case; a computer peripheral such as a keyboard or mouse; furniture such as a chair, couch or recliner; audio equipment such as headphones; eyewear; clothing; footwear; a container such as a beverage or food container.

In certain embodiments, the non-invasive device comprises a communication device. The communication device can be a communication transmitter or communication receiver to transmit or receive data. In these embodiments, the communication device can transmit or receive data with a wireless or cellular network. Advantageously, the communication device can transmit the raw impedance data to a remote or cloud-based computer such as a supercomputer, base station, server or another device such as a smart phone, laptop or tablet to compute and determine blood glucose concentration remotely. In this embodiment, the blood glucose concentration of a subject can be monitored even without access to a computer or phone, such as children, the elderly or at-risk individuals. This could be used to provide an alarm to a remote user that the subject had passed a pre-determined blood glucose concentration threshold. In other embodiments, the computation can be processed by the non-invasive device and the data can be transmitted to a remote or cloud-based computer.

In use, the non-invasive device is worn such that the electrodes make conductive skin contact with the subject. For example, the skin site can be located on the volar forearm, down to the wrist, behind an ear, on an ear, on an earlobe, or the finger of a subject. In some cases, the skin can be pre-treated, such as using a saline or alcohol solution (such as isopropanol solution) or shaved, prior to the measuring step or before being worn. An electrically conductive gel can be optionally applied to the skin to enhance the conductive contact of the electrodes with the skin surface during the measuring step.

The electrodes can be in operative connection with a microprocessor programmed to determine the amount of blood glucose based upon the measured impedance. There can be an indicator operatively connected to the microprocessor for indication of the determined amount of blood glucose to the subject. The indicator can provide a visual display to the subject.

In certain embodiments, the microprocessor can be operatively connected to an insulin pump and the microprocessor is programmed to adjust the amount of insulin flow via the pump to the subject in response to the measured amount of blood glucose.

The microprocessor can be programmed to compare the measured impedance with a predetermined correlation between impedance and blood glucose concentration. The non-invasive device can include a receiver for measuring impedance at a plurality of frequencies.

In operation, the non-invasive device can calibrate the device against a directly measured glucose concentration of a subject. The device can input the value of the directly measured glucose concentration in conjunction with impedance measured about the same time, for use by the operating software to determine the blood glucose level of that subject at a later time based solely on subsequent impedance measurements.

In some embodiments, data produced by the non-invasive device can be collected, stored (for example remotely), and compiled for analysis.

According to another aspect, the present invention provides a method for non-invasively determining blood glucose concentration in a subject, the method comprising the steps of:
measuring impedance through a portion of the subject using at least one electrode in conductive contact with the subject's skin; and
determining the amount of blood glucose in the subject based upon the measured impedance,
wherein the at least two electrodes are in a configuration which passes electrical current through the portion of the subject.

According to a further aspect, the present invention provides a method for non-invasively determining blood glucose concentration of in a subject, the method comprising the steps of:
measuring impedance through a portion of the subject using at least two electrodes in conductive contact the subject's skin;
determining the amount of blood glucose in the subject based upon the measured impedance; and
measuring at least one additional physiological parameter of the subject.

It should be appreciated by the skilled addressee that any suitable frequency can be used to measure the impedance. In some embodiments, the impedance is measured at a plurality of frequencies. In some embodiments, the amount of blood glucose concentration is determined by determining the ratio of the impedance at a plurality of frequencies, such as the ratio of two frequencies. In certain embodiments, the method is performed at a frequency range of between about 0.1 Hz to about 1 MHz, between about 5 Hz to about 1 MHz, between about 20 Hz to about 1 MHz, between about 5 Hz to about 800 kHz, between about 5 Hz to about 500 kHz, between about 2 Hz to about 500 kHz.

In some embodiments, the method of the present invention is performed using alternating current (AC). In some embodiments, the method of the present invention is performed using direct current (DC).

In some embodiments, the portion of the subject is a body part of a subject. In some embodiments, the portion of the subject is selected from the group consisting of a finger, an ear, a waist, a leg, an arm, a wrist and combinations thereof.

In certain embodiments, the method of the present invention is continuous. In some embodiments, the method of the present invention is measured at intervals. In some embodiments, the duration of each single measurement of blood glucose concentration is between about 2 seconds to about 10 minutes, between about 2 seconds to about 5 minutes, between about 2 seconds to about 3 minutes, between about 2 seconds to about 2 minutes. In some embodiments, the duration of each single measurement of blood glucose concentration is less than about 10 minutes, less than about 5 minutes, less than about 3 minutes, less than about 90 seconds, less than about 60 minutes, less than about 30 seconds. In some embodiments, the duration of each single measurement of blood glucose concentration is less than 48 hours, less than 30 hours, less than 24 hours, less than 12 hours, less than 8 hours, less than 4 hours, less than 2 hours, less than 1 hour.

In some embodiments, the method of the present invention measures impedance at intervals between about 2 seconds to 60 minutes, between about 2 seconds to 30 minutes, between about 2 seconds to 10 minutes, between about 2 seconds to 5 minutes, between about 2 seconds to 3 minutes, between about 2 seconds to 1 minute, between about 2 seconds to 30 seconds, between about 2 seconds to 15 seconds, between about 2 seconds to 10 seconds, between about 2 seconds to 5 seconds. In some embodiments, the method of the present invention measures impedance at about 2 seconds, about 5 seconds, about 10 seconds, about 15 seconds, about 30 seconds, about 1 minute, about 3 minutes, about 5 minutes, about 10 minutes. In some embodiments, the method of the present invention measures impedance continuously or repeatedly to provide substantially continuous measurements at intervals.

In certain embodiments, the method of the present invention further comprises measurement of at least one additional physiological parameter of a subject. In certain embodiments, the physiological parameter (i.e., biometric) is selected from the group consisting of body fat, muscle mass, body composition, body temperature, skin pH, skin temperature, blood pressure, heart rate and combinations thereof.

In some embodiments, the method comprises a pre-treatment step, wherein the pre-treatment step involves shaving and/or cleaning the skin. The skin can be cleaned with a saline or alcohol (such as isopropanol solution) solution prior to the measuring step or before being worn. In some embodiments, an electrically conductive layer (such as in the form of a gel, paste, ointment or cream) can be applied to the skin to enhance the conductive contact of the electrodes with the skin surface during the measuring step.

In certain embodiments, the method of the present invention comprises use of an artificial neural network. In certain embodiments, the method of the present invention comprises use of an artificial neural network (ANN) to process the impedance signal to improve signal quality. In certain embodiments, the method of the present invention comprises use of an artificial neural network to perform a non-linear regression. In certain embodiments, the method of the present invention comprises use of an artificial neural network to predict and/or determine blood glucose concentration of a subject. In certain embodiments, the artificial neural network (ANN) model correlates the measured biometrics (including but not limited to bioimpedance, body temperature, skin pH, blood pressure and the like) to blood glucose concentration. In certain embodiments, a different ANN architecture or model can be used depending on the form factor of the non-invasive device such as whether the device is a ring, a bracelet, a smart watch or other form). In certain embodiments, the method of the present invention comprises a dynamic adaptive ANN. In this embodiment, the dynamic adaptive ANN enables the non-invasive device to adapt to the specific physiological parameter patterns of the subject which increases the accuracy of the blood glucose concentration measurement while in use and being worn by the subject.

As discussed previously, the present invention provides a non-invasive device which can measure impedance with high-quality signals. This enables a user (which can also be the subject) to monitor the quality of the output electrical current signals before using the data to determine the blood glucose concentration. This allows selection of quality data by removing the noisy and low-quality signals and only using the high-quality data for the ANN to increase the accuracy or enable the ANN's functionality when determining the blood glucose concentration.

According to another aspect, the present invention provides a kit comprising:
at least two electrodes adapted to be connected to a receiver for measuring an impedance signal; and
a housing adapted to receive the electrode.

In some embodiments, the kit comprises a receiver. In some embodiments, the receiver is an electrochemical impedance spectroscopy (EIS) device. In some embodiments, the kit comprises an insulin pump.

### Definitions

In describing and claiming the present invention, the following terminology will be used in accordance with the definitions set out below. It is also to be understood that the terminology used herein is for the purpose of describing particular embodiments of the invention only and is not intended to be limiting. Unless defined otherwise, all technical and scientific terms used herein have the same meaning as commonly understood by one having ordinary skill in the art to which the invention pertains.

Unless the context clearly requires otherwise, throughout the description and the claims, the words “comprise”, “comprising”, and the like are to be construed in an inclusive sense as opposed to an exclusive or exhaustive sense; that is to say, in the sense of “including, but not limited to”.

As used herein, the phrase “consisting of” excludes any element, step, or ingredient not specified in the claim. When the phrase “consists of” (or variations thereof) appears in a clause of the body of a claim, rather than immediately following the preamble, it limits only the element set forth in that clause; other elements are not excluded from the claim as a whole. As used herein, the phrase “consisting essentially of” limits the scope of a claim to the specified elements or method steps, plus those that do not materially affect the basis and novel characteristic(s) of the claimed subject matter.

With respect to the terms “comprising”, “consisting of”, and “consisting essentially of”, where one of these three terms is used herein, the presently disclosed and claimed subject matter may include the use of either of the other two terms. Thus, in some embodiments not otherwise explicitly recited, any instance of “comprising” may be replaced by “consisting of” or, alternatively, by “consisting essentially of”.

Other than in the operating examples, or where otherwise indicated, all numbers expressing quantities of ingredients or reaction conditions used herein are to be understood as modified in all instances by the term “about”, having regard to normal tolerances in the art. The examples are not intended to limit the scope of the invention. In what follows, or where otherwise indicated, “%” will mean “weight %”, “ratio” will mean “weight ratio” and “parts” will mean “weight parts”.

The term “substantially” as used herein shall mean comprising more than 50% by weight, where relevant, unless otherwise indicated.

The recitation of a numerical range using endpoints includes all numbers subsumed within that range (e.g., 1 to 5 includes 1, 1.5, 2, 2.75, 3, 3.80, 4, 5, etc.).

The terms “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the invention.

It must also be noted that, as used in the specification and the appended claims, the singular forms “a”, “an” and “the” include plural referents unless the context clearly dictates otherwise.

The prior art referred to herein is fully incorporated herein by reference.

Although example embodiments of the disclosed technology are explained in detail herein, it is to be understood that other embodiments are contemplated. Accordingly, it is not intended that the disclosed technology be limited in its scope to the details of construction and arrangement of components set forth in the following description or illustrated in the drawings. The disclosed technology is capable of other embodiments and of being practiced or carried out in various ways.

### BRIEF DESCRIPTION OF THE DRAWINGS

A preferred embodiment(s) of the invention will now be described, by way of example only, with reference to the accompanying drawings(s) in which:

FIG. 1 shows the setup of Keysight E4990A to measure impedance of a human arm using ImpediMed gel electrodes.

FIG. 2 shows the result of a 4T (four-electrode) human arm measurement using the Inphaze system compared to ImpediMed SFB7.

FIG. 3 shows an embodiment of a non-invasive device in the form of a wearable ring having eight apertures adapted for receiving up to 8 electrodes.

FIG. 4 shows gold plated copper electrodes.

FIG. 5 shows an embodiment of a non-invasive device in the form of a wearable ring having eight electrodes.

FIG. 6 shows different housing configurations for the non-invasive device which were 3D printed.

FIG. 7 shows a 4T (four electrode) non-invasive device in the form of a wearable ring having an alternative configuration.

FIG. 8 shows the result of 4T ring measurements over 7 repeats having the embodiment of FIG. 7. a) average impedance vs frequency, b) average phase vs frequency, c) average conductance vs frequency, and d) average capacitance vs frequency.

FIG. 9 shows the measurement signal quality of the ring embodiment of FIG. 7.

FIG. 10 shows a fully assembled non-invasive device in the form of a bracelet.

FIG. 11 shows 4T bracelet measurement (4 repeat runs) using an EIS of the embodiment of FIG. 10. a) average impedance vs frequency), and b) average phase vs frequency.

FIG. 12 shows 4T bracelet measurement (4 repeat runs) using an Inphaze system (4 repeat runs) of the embodiment of FIG. 10. a) average impedance vs frequency), and b) average phase vs frequency.

FIG. 13 shows the measurement signal quality of the bracelet embodiment of FIG. 10.

FIG. 14 shows a 4-Terminal (4T) configuration of a non-invasive device in the form of a wearable ring to be used for human trials.

FIG. 15 shows the measurement signal quality of 4T ring of the embodiment of FIG. 14.

FIG. 16 shows 4T impedance wrist measurements using ImpediMed SFB7 and ImpediMed gel electrodes.

FIG. 17 shows electrical interferences on bioimpedance signals.

FIG. 18 shows an embodiment of a Faraday cage for bioimpedance measurements.

FIG. 19 shows the effect on impedance signals between before and after introducing electrical interference on resistors, showing no interference.

FIG. 20 shows a) ring impedance measurements outside the Faraday cage with electrical interference nearby and b) ring impedance measurements inside the Faraday cage with electrical interference placed directly on top and side of the Faraday cage.

FIG. 21 shows results from a first clinical oral glucose tolerance test (OGTT) performed on participant 1 comparing the clinical blood glucose concentration to the blood glucose concentration measured using Accu-Chek and FreeStyle Libre devices.

FIG. 22 shows results from the second clinical oral glucose tolerance test (OGTT) performed on participant 1 comparing the clinical blood glucose concentration to the blood glucose concentration measured using Accu-Chek and FreeStyle Libre devices.

FIG. 23 shows results from the clinical oral glucose tolerance test (OGTT) performed on participant 2 comparing the clinical blood glucose concentration to the blood glucose concentration measured using an Accu-Chek device.

FIG. 24 shows a Clarke Error Grid (CEG).

FIG. 25 shows a Parkes Error Grid (PEG) for type 1 (A) and type 2 (B) diabetes.

FIG. 26 shows a Surveillance Error Grid (SEG).

FIG. 27 shows examples from the literature of the ranges of BGLs for different participant groups.

FIG. 28 shows a PEG plot (type 1 diabetes) showing the early Accu-Chek and FreeStyle Libre data collected from participant 1 and participant 2. The range on the x- and y-axes has been adjusted for better visualisation.

FIG. 29 shows early bioimpedance results through the full body using the ImpediMed device. Shown are measurements made at 3 distinct timepoints, where 5 measurements were made at each of timepoint.

FIG. 30 shows ImpediMed gel electrode configurations used to measure bioimpedance through the a) upper-side, b) under-side of the forearm and c) through the finger.

FIG. 31 shows typical Cole plots for bioimpedance measurements made through (A) the full body, (B) the forearm (upper-side and underside), and (C) the finger using the ImpediMed device.

FIG. 32 shows sites of electrode placement on (a, b) participant 1 and (c, d) participant 2 for bioimpedance measurements through the full body using the ImpediMed device.

FIG. 33 shows plots comparing each blood glucose concentration (BGL) measurement (Accu-Chek) with the bioimpedance value (ImpediMed) at 51.172 Hz for (A) participant 1 and (B) participant 2. Shown are all 5 measurements taken during a single bioimpedance measurement. Where multiple measurements were taken at the same BGL value, a different colour/symbol (BGL #1-#5) was used to distinguish between these.

FIG. 34 shows an example of a Parkes Error Grid (PEG) plot.

FIG. 35 shows a) EIS instrument, b) prototype dry electrodes in a ring housing used for c) bioimpedance measurements through the finger.

FIG. 36 shows sites of electrode placement on participant 1 for bioimpedance measurements (a, b) through the full body or (c, d) through the wrist.

FIG. 37 shows sites of electrode placement on participant 2 for bioimpedance measurements (a, b) through the full body or (c, d) through the wrist.

FIG. 38 shows sites of electrode placement on participant 3 for bioimpedance measurements (a, b) through the full body or (c, d) through the wrist.

FIG. 39 shows sites of ring and electrode placement on participant 2 for bioimpedance measurements through the finger.

FIG. 40 shows photographs of each stage of human testing. Shown are the following measurements: bioimpedance through (a) the full body, (d) the wrist, and (e) the finger, skin temperature across (b) the full body, (c) the wrist, and (f) the finger, BGL made with (g, i) Accu-Chek and (h) FreeStyle Libre, (j) blood pressure and heart rate, and skin pH across (k, l) the full body, (m) the wrist, and (n) the finger.

FIG. 41 shows a photograph exemplifying correct lead placement for bioimpedance measurements.

FIG. 42 shows a flowchart for determining clinical trials of medical devices. BGL stands for blood glucose concentration/level, and BI stands for bioimpedance. ¹At least one component of the testing rig is not TGA approved. For example, using a non-medical EIS machine with prototype electrodes, a TGA approved EIS machine with prototype electrodes, or any other combination. ²This product is ready for sale except that it does not have TGA approval at this stage.

FIG. 43 shows (a) top and (b) bottom view of an alternative electrode configuration for an embodiment of a non-invasive device in the form of a ring.

FIG. 44 shows (a) 8 different configurations of electrodes with arrows showing current flow path and (b) the bioimpedance data of the 8 configurations for an embodiment of a non-invasive device in the form of a ring.

FIG. 45 (a) and (b) show alternative configurations of embodiments of a non-invasive device in the form of a ring or wearable device.

FIG. 46 shows a representative bioimpedance result for a square and circular electrode for an embodiment of a non-invasive device in the form of a ring.

FIG. 47 shows a representative bioimpedance result using electrodes of different sizes for an embodiment of a non-invasive device in the form of a ring.

FIG. 48 shows the effect of electrode spacing on bioimpedance measurements for an embodiment of a non-invasive device in the form of a ring.

### DETAILED DESCRIPTION OF THE INVENTION

The skilled addressee will understand that the invention comprises the embodiments and features disclosed herein as well as all combinations and/or permutations of the disclosed embodiments and features.

### EXAMPLE 1

### Electrical Impedance Spectroscopy (EIS) Instrument Validation and Selection

For impedance measurements on a subject, preferably, a human subject, a non-invasive device will be worn and the device will collect bioimpedance data in intense sessions.

An embodiment of the device consists of 2 main parts: a front-end where electrodes will be “worn” by the test participant in order to make electrical contact with the skin; and a back-end where an EIS instrument will collect bioimpedance data of the subject via the electrodes. The main design considerations include:
Front-end: Placement of the electrodes; method of securing the electrodes; electrode contact area; wet or dry contact with the skin; materials to be used; and
Back-end: Resolution of the EIS instrument; frequency range of interest for measurement; observed impedance range; reliability of bioimpedance measurements in complex moving systems such as the human body.

In order to determine whether using non-invasive bioimpedance measurements through skin contact would provide distinguishable readings to different blood glucose concentrations, the present inventors required an EIS instrument with good accuracy, a wide measurement frequency range and a wide measurable impedance range. As for measurement frequencies, there are no precise studies from literature showing what information could be obtained at lower frequencies. Therefore, for the selection criteria, low frequencies were measured, and can be later processed to determine its usefulness. For measurements below 1 Hz, a long duration is required to capture the data which may be unrealistic for a non-invasive device. At higher frequencies the intracellular and extracellular electrolytic solutions in the body can act as a short for impedance measurements, therefore the required measurement frequency should not need to exceed beyond the kHz, preferably 1 MHz range.

Table 1 summarises some of the general-purpose EIS instruments available on the market today.

TABLE 1
Shortlisted EIS instrument available on the
market for consideration of this project.

| | | Keysight | Newton4th | |
|---|---|---|---|---|
| | | E4990A-010 | PSM1735 + IAI | |
| Brand/ | Solartron | (Keysight | (Pacific Test | |
| Model | 1260A | Technologies | Equipment | BioLogic |
| (Distributor) | (Ametek) | Australia) | Pty Ltd) | MTZ-35 |
| Price | USD | AUD $20,883 + | AUD $16,031 + GST | AUD $45,990 |
| | $33,550 =>> | GST Refurbished | (PSM1735 + IAI) | AUD + GST |
| | AUD | | | |
| | $48,550 | | | |
| Leadtime | — | 2-3 Weeks | 4-5 Weeks | 6-8 Weeks |
| Measurement | 10 μHz to 32 MHz | 20 μHz to 10 MHz | 10 μHz to 35 MHz | 10 μHz to 35 MHz |
| Frequency | | | | |
| Range | | | | |
| Measurable | 10 mΩ-100 MΩ | 25 mΩ-40 MΩ | 10 mΩ-100 MΩ | 1 mΩ-500 MΩ |
| Impedance | | | | |
| Range | | | | |

EIS instruments that met criteria were: Solartron 1260A, Keysight E4990A-010, Newton4th PSM1735+IAI and BioLogic MTZ-35.

After consideration of cost, performance and lead-time, Keysight E4990A was selected as the main general-purpose EIS instrument for the non-invasive device. For additional bioimpedance measurements that are purposely-built to give body composition readings such as fat-free mass (FFM), fat mass (FM), total body water (TBVV), intracellular fluid (ICF), extracellular fluid (ECF), the ImpediMed SFB7 were used. The ImpediMed SFB7 could also be used to make general-purpose EIS measurements but the frequency range was limited to 4 KHz to 1 MHz and the measurable impedance range is below 1.1 KΩ.

The testing protocol of the Keysight E4990A-010 general-purpose EIS instrument was as follows:
1. Measuring a high-precision resistor and determine its performance.
Performance validation: determine how close the measured impedance, |Z|, is to the resistor value, and how close the measured phase was to zero which is the theoretical phase value for a pure resistor independent of frequency.
2. Measure a known Max-Wagner (MW) circuit as the sample and determine its performance. A Max-Wagner circuit is made up from multiple R//C (resistor//capacitor) elements that are in series.
Performance validation: determine how close the reconstructed circuit (from using measured EIS data) was to the known circuit.
3. Measure impedance of a human arm using gel electrodes from ImpediMed (ImpediMed 292-STE) in a 4-terminal configuration.
Performance validation: comparing the results to the same arm measurement obtained using ImpediMed SFB7.

### Performance of EIS Instruments

Keysight E4990A

Test Circuit

The Keysight E4990A comes with a calibration certificate and a 100Ω test box. The test box was firstly used to become familiar with the system and determine its performance.

When measuring resistors or MW circuits using 1-meter cables, significant Z and phase errors were observed. After conducting phase and load compensation as per instructed in the Four-terminal pair configuration section of the manufacturer's Impedance Measurement Handbook (Keysight Technologies 2016), the performance of the system was improved. With a few more test runs on known samples such as resistors and MW circuits, the Keysight E4990A instrument was validated and deemed to be performing well as a general-purpose EIS system which provided a measurement frequency range of 20 Hz to 1 MHz.

Gel Electrodes

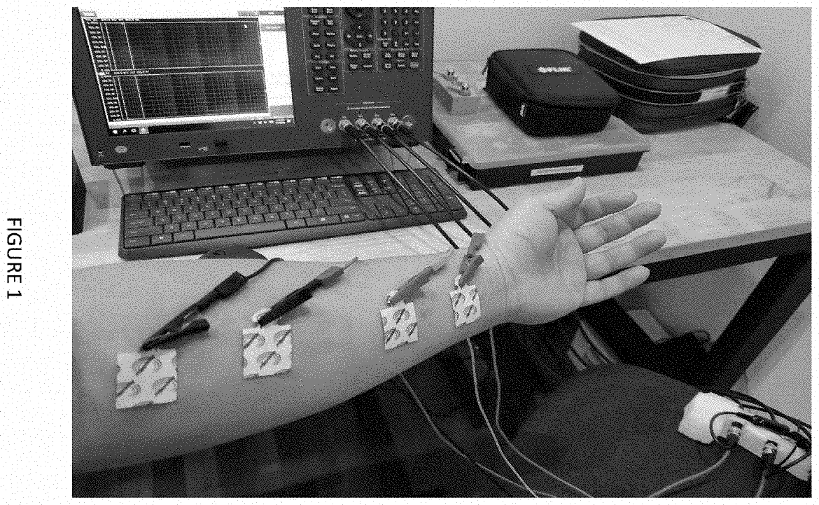

The Keysight E4990A instrument was then used to measure the impedance of a human arm using ImpediMed gel electrodes. The Keysight E4990A instrument was deemed very low risk when generating zero DC bias and an AC amplitude of 1 V maximum. The ImpediMed gel electrodes were used because they are already FDA (US Food and Drug Administration) and TGA (Australian Therapeutic Goods Administration) approved and the results could be compared to that obtained using the ImpediMed SFB7 instrument. The setup is shown in FIG. 1.

ImpediMed SFB7

Test Circuit

Due to the limited measurable impedance range of the ImpediMed SFB7 instrument (up to 1 KΩ), a 100Ω resistor was measured for validation, not Max-Wagner circuits. The ImpediMed SFB7 was used primarily for its intended usage in this application which provides body composition measurements.

Inphaze High Resolution EIS

Test Circuit

The Inphaze EIS instrument is a general-purpose EIS system. It was designed for making high resolution measurements and therefore the measurement time is long. A typical 1 Hz to 1 MHz scan (3 spectra) takes approximately 10 minutes. Due to its useful capability to explore samples with unknown impedances, it was used for evaluating various electrode designs for the non-invasive device of the present invention. Wherever comparable, the Inphaze system was also used to cross-validate results from other EIS devices.

The Inphaze Impedance Analyser software was used to automatically reconstruct Max-Wagner circuits and also to plot the impedance, phase and Nyquist curves. Converter utilities were developed to convert data files generated by Keysight E4990A and ImpediMed SFB7 into the “.izx” file format which is compatible with the Inphaze Impedance Analyser software.

Gel Electrodes

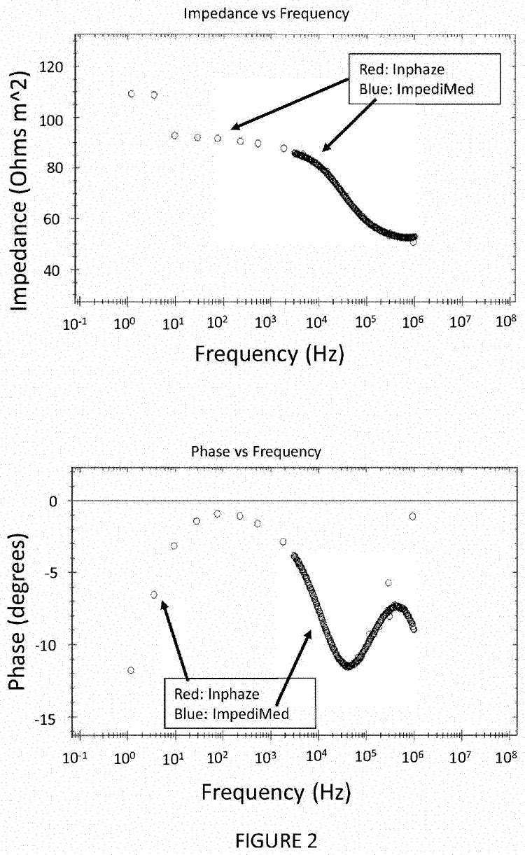

Measurements using the Inphaze system on a human subject was deemed very low risk when the DC bias is zero and the AC amplitude is 1 V maximum. FIG. 2 shows the result of the 4T (four electrode) human arm measurement obtained by both the Inphaze instrument and ImpediMed SFB7. A good overlap can be observed in general but ImpediMed SFB7 was giving a large phase error at higher frequencies and this can be confirmed by its 100Ω resistor validation run (2 degrees error). Further, the shape of the phase profile from the Inphaze measurement (as shown in FIG. 2) appears closer to what one would expect in this type of multi-layer (electrically equivalent) sample.

EIS Instrument

The present inventors also used an EIS system that had the same working theory to the Inphaze system.

Test Circuit

The EIS solution had a very similar performance to the Inphaze high resolution system. The measurement time to scan from 1 Hz to 1 MHz (3 spectra) was in the order of about 1-2 minutes, which was suitable for measurements in human subjects.

Gel Electrodes

Similar to the Keysight E4990A and Inphaze instruments, the EIS instrument for measurement on human subjects was deemed very low risk when the DC bias was zero and the AC amplitude was 1 V maximum. Two of the unique features of both the Inphaze system and the system were (i) the ability to observe the actual measurement AC waveforms and (ii) see the real-time signal-to-noise ratio (SNR) value in the data acquisition software. This enabled us to see the quality of the electrodes, if they were making contacts properly, if they were causing distortions in the signal, or if there were interferences that cause distortions in the signal. The waveforms (not shown) indicated very clean signals with no distortion and also the SNR values in the measurement data was very good.

EIS systems that were general-purpose and sufficiently accurate to explore various non-invasive device (wearable) configurations (materials, placement, surface area and the like) with unknown impedances and unknown frequency ranges of interest were evaluated. Several EIS systems on the market were evaluated and the Keysight E4990A instrument was chosen. The inventors also used an EIS system that performed well for this application and met all the requirements. Additionally, the system featured a very useful utility to see the actual measurement signal waveforms and SNR real-time, assisting in assessing electrode performance.

### EXAMPLE 2

### Electrode Design

Research and development was undertaken to design a suitable front-end for the non-invasive wearable device prototype for human subjects. The front-end of the non-invasive wearable device is where EIS electrodes make contact with test participants in order to collect bioimpedance data non-invasively via skin. Design considerations for such development included: placement of the electrodes; securing of the electrodes; electrode contact area; wet or dry contact with the skin; material to be used, etc. These factors can affect the ability of the electrodes to measure a subject's blood glucose concentration by correlating non-invasive bioimpedance measurements.

Electrode Design Considerations

Number of Electrodes

The non-invasive devices used for impedance analysis had 4 channels (2 for current and 2 for voltage). When making EIS measurements, the key benefit of separating the current injecting electrodes from the voltage sensing electrodes was that any loading or polarisation of the current injecting electrodes would not affect the voltage sensing performance. There should be no current flowing in or out of the voltage sensing paths as only the voltage, or potential, response of the sample due to the stimulating current should be sensed. Optionally, an additional reference electrode to address signal drift issues if encountered could be used.

Electrode Placement

Bioimpedance measurements are typically performed over large segments (i.e., surface area) on the body, however some devices have functioned on smaller areas such as the wrist. Obtaining a high-quality signal requires good contact over as large a possible surface area. However, this may need to be balanced depending on the form factor. For example, if a ring is desired as the non-invasive wearable device, the size of the electrode will be determined by the minimum electrode size that can obtain a high-quality signal.

Electrode Orientation

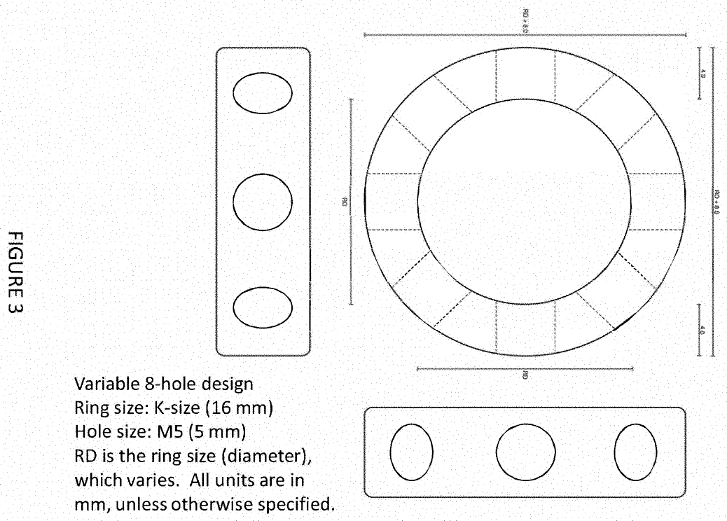

The current source and sink were placed on opposing ends of the wearable. Electrodes that could be selected as current source/sink (C) or voltage sense (V) were considered in order to ensure the measurement was reliable and accurate. This configuration is shown in FIG. 3, where any of the 8 positions could be used to insert electrodes. Ideally voltage sense would be placed at points between the two regions of current stimulation.

The electrodes could all be facing the finger, or electrode(s) may be placed on the exterior in order to facilitate a path from ring exterior, to right hand, to chest, to left hand, to ring interior. A reference voltage may also be useful for drift correction.

Manufacturing

The present inventors manufactured electrodes for the present invention. The ideal electrode specifications were small, dry and could be placed in a housing.

Wet/Gel Electrode Contact

For both bioelectrical monitoring (EEG, ECG) and stimulating (FES, tES, TENS) purposes, the use of gel electrodes to maintain contact can be used. In regard to stimulation, this is the result of gel electrodes typically exhibiting less broadband noise in contrast to dry electrodes. In regard to assessing the viability of bioimpedance corresponding to blood glucose concentration, gel electrodes are excellent as they remove any unknowns in measurements due to factors that may influence dry contact electrodes.

Gel electrodes were ordered from the same manufacturer and were assessed for consistency and reproducibility as well as to assess the viability of measuring any meaningful signal from relatively localised regions of the body (such as the forearm or a finger) when validating the development of the non-invasive device of the present invention.

Electrode Materials

Base Materials (Singular)

Materials of a relatively uniform composition were considered for their potential ease-of-use in manufacturing. Several materials were examined for their efficacy as electrodes. Electrode materials are listed in Table 2 as potentially being suitable for electrode-skin contact. The entire electrode can be composed of the same material at the proof-of-concept stage, potentially simplifying the manufacturing process.

TABLE 2
Materials considered for electrode surface contact.

| | | | Oxidises/ | |
|---|---|---|---|---|
| | Relative | Relative | Interacts | |
| Material | Cost^{a} | Conductivity | with Skin | Comments |
| Gel | $ | Very High | No | Good contact, |
| Ag/AgCl | | | | need to reapply |
| | | | | regularly, wet |
| | | | | electrode |
| Copper | $ | Very High | Yes | |
| Silver | $$ | Very High | No | |
| Conductive | $ | Low | No | Exact conductivity |
| Rubber | | | | varies |
| Conductive | $$ | Low | No | Exact conductivity |
| Carbon | | | | varies |
| Platinum | $$$ | High | No | |
| Stainless | $$ | Low | No | |
| Steel | | | | |
| Titanium | $$ | Low | No | |
| Gold | $$$ | High | No | |
| Aluminium | $ | Medium | Yes | |

^{a}$ = lowest cost, $$ = intermediate cost, $$$ = highest cost.

As seen in Table 2, there are a few important attributes associated with the materials. For example, copper oxidises readily with the skin when an electrical current is applied, jeopardising the repeatability of measurements. It is for this reason that copper was not considered as a direct electrode contact material. On the other hand, elements like gold are particularly suitable due to their non-reactivity, however the cost of producing a singular piece of gold is expensive. As gold was desired as a contact material, alternative methods of coating cheaper conductive materials were examined.

Sputter-Coating

Sputter-coating was investigated. This method was cheaper than using pure gold as the electrode material and would enable a wider variety of electrode shapes.

Electroplating

Electroplating gold onto other conductive materials was investigated. Several base materials were considered, including aluminium, stainless steel and copper. Soft-plating of 24K gold was chosen over hard plating, as soft plating has a purer gold content on plate, despite being thinner. Typically, medical applications use soft plating for skin contact due to higher purity. Primarily this coating method was considered due to the high price of gold.

The only issue encountered with electroplating would be the quality of the plated product. Likely due to issues with plating techniques, this varied between well plated material (high reflective appearance) and “dulled” plated material. Some of the finishes were scratch-prone, whereas others had a very robust finish.

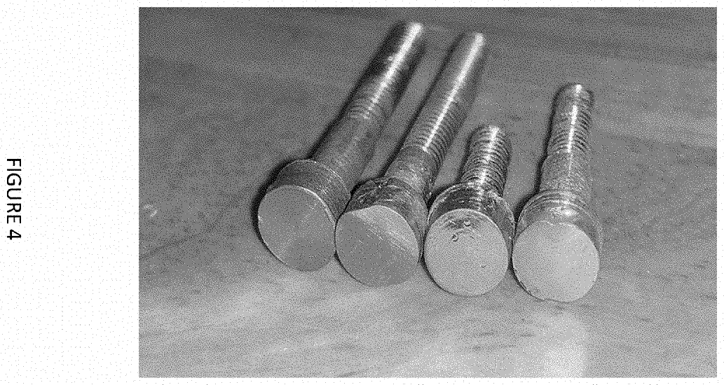

FIG. 4 shows some variability in the plating process, particularly on the contact side (closest to the camera).

Adhesion

Adhesion coating was also considered. Most adhesive options were placed between a conductive metal and gold sheet. Silver epoxy, conductive paint and similar materials were used. Some of these conductive adhesives were not durable, however, the durability can be optimised and improved. Solder was the best performing in terms of durability for joining metals when coating electrodes of the present invention. When using gold plate and copper wires, the two were joined and then secured with glue on a hollow plastic (nylon) screw.

Comparison Between Coating Methods and Materials

The two electrode designs that appeared to have the most promise were gold electroplated copper and gold electroplated on a nylon screw with a banana connector on one end and gold sheet on the other.

In relation to the prototyping and subsequent stages of product development of the non-invasive device, the contact electrode material is an important component. However, the gold electroplated on a nylon screw with a banana connector configuration was selected for further validation because given that the electrode material is pure gold, any risk of imperfect coverage of copper was alleviated.

Electrode Requirements

Repeatability of Use

Repeatability of measurements is important, particularly in this initial stage where the importance of relevant variations in the signal are unknown. Therefore, robust electrodes were needed for further validation of impedance measurements for blood glucose concentration determination. Particularly, electrodes that could withstand several months of testing without significant variations due to changes in the electrode were desired.

Size

The selection of an appropriate electrode size has two competing factors. From a physics standpoint, the electrode-skin contact area should be as large as permissible, from a wearable standpoint it should be as small as possible.

For the embodiment of a ring as the non-invasive device, electrode contact areas of between 19 mm² and 36 mm² were considered for each electrode. This was for either circular or square configurations between approximately 5 mm and 6 mm. These sizes were chosen as they were large enough to produce a signal, but small enough as to not overlap for a range of potential standard ring sizes. IEC 60601 provides international standards, limiting current for DC and AC frequencies less than 1 kHz to 10 μA, and for AC currents above 1 kHz as per equation 1 discussed above.

### EXAMPLE 3

### Housing Requirements

Similar Devices on the Market

As a point of comparison, a selection of commercially available smart rings were procured. This included the Motiv and GO2SLEEP smart rings. The Motiv ring tracks various metrics around fitness, whereas the GO2SLEEP ring tracks metrics associated with sleep quality. Another similar product (non-invasive, glucose monitor) that could not be procured was the GlucoTrack that performs measurements with an ear clip.

Full Body Analysis

Full body analysis was examined for bioimpedance measurements of the present invention. Standard EIS measurements were taken on the full body using the ImpediMed device.

Localised

A non-invasive device that measured a localised region of the body was examined. The electrode configurations tested were typically on areas of the forearm, hand or finger. This can be ideal as a localised non-invasive device can be passive in operation by the user.

### EXAMPLE 4

### Wearable Prototypes

Ring

Design

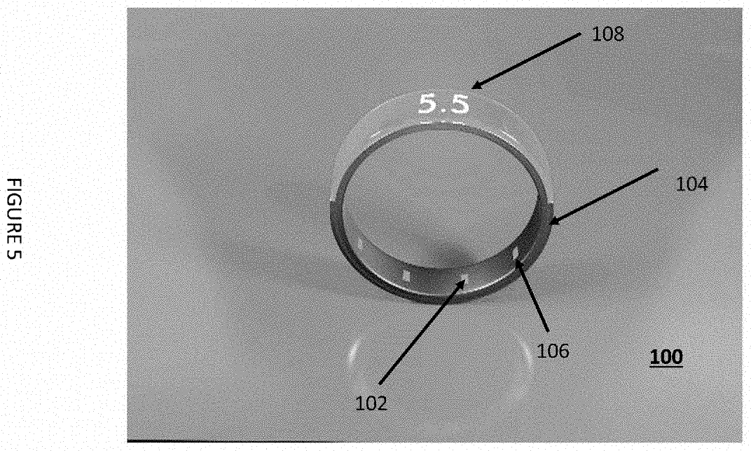

The non-invasive device as a wearable ring in one embodiment of the present invention. Given the rings small form factor, the success of its functionality was most desired in contrast to the other wearable designs. FIG. 5 shows the representation of a ring device.

Referring to FIG. 5, the non-invasive device 100 in the form of a ring comprises eight electrodes 102 made of gold for contacting skin. The electrode 102 is adapted to be connected to a receiver (not shown) which is housed within the ring 100 to process the impedance signals. A housing 104 in the form of a ring has eight apertures 106 to receive the eight electrodes 102. The electrodes are configured and spaced apart at about 45° around the internal periphery of the ring 100 such that in use an electrical current passes through a portion of a subject (i.e., a finger) in use.

In this embodiment, four electrodes are current injecting (stimulating) electrodes and four electrodes are voltage measurement (sensing) electrodes to measure impedance.

In use, a battery (not shown) is placed in the housing 104. The battery can be non-rechargeable and installed/removed through a slot of the housing 104. In other configurations, a rechargeable battery can be used which is integral to the ring 100. A charging and/or data port (not shown) can be connected to the ring 100 to allow for charging and/or sharing data with a mobile electronic device such as a computer, tablet or smart phone.

The ring 100 has a notification indicator 108 to display the blood glucose concentration as well as other physiological parameters.

In use, the device can also in some embodiments wirelessly transfer data to the mobile electronic device such as a smart phone for external signal processing and measurements.

A total of 15 distinct ring designs were trialled in this example by the present inventors. These designs can be grouped into 6 separate major design revisions, with design similarities between a few of the designs.

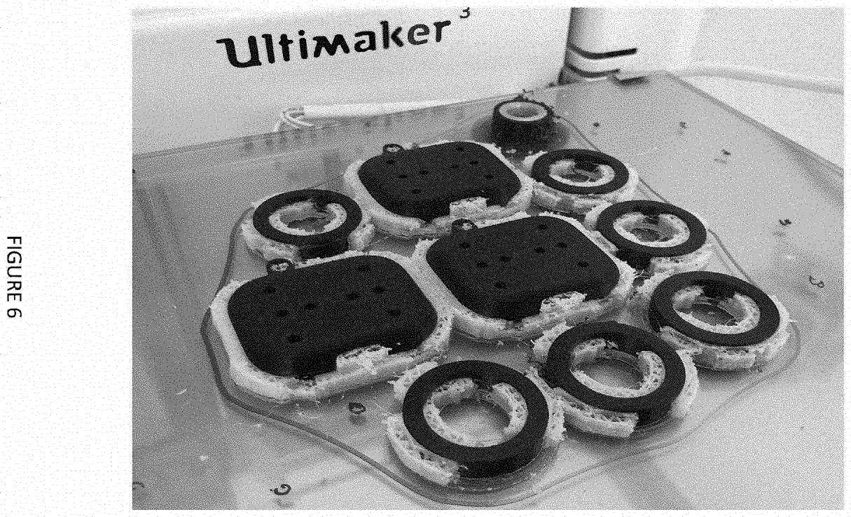

The first design incorporated 8 holes with later designs focusing on the required number of electrodes and different angle offsets. Later designs included space for a thermocouple. The later designs focused on electrode placement instead of ring sizing, as was the case for FIGS. 5 and 6.

FIG. 6 shows different designs for wearable housings which were 3D printed for rapid-prototyping.

Manufacturing and Assembly

The exact manufacture process of the housing was typically dependant on the electrode it was adapted to fit. Typically, different housing configurations were printed on the Ultimaker 3 3D printer in Black CPE+ with water-soluble polyvinyl alcohol (PVA) supports. Dependent on the electrode to be used and inserted, premade holes may undergo subsequent threading. Subsequently, the ring may be sanded and refined. The exact manufacturing process is dependent on which ring model is to be manufactured. Following manufacture of the housing, the relevant electrodes can then be inserted.

EIS Performance

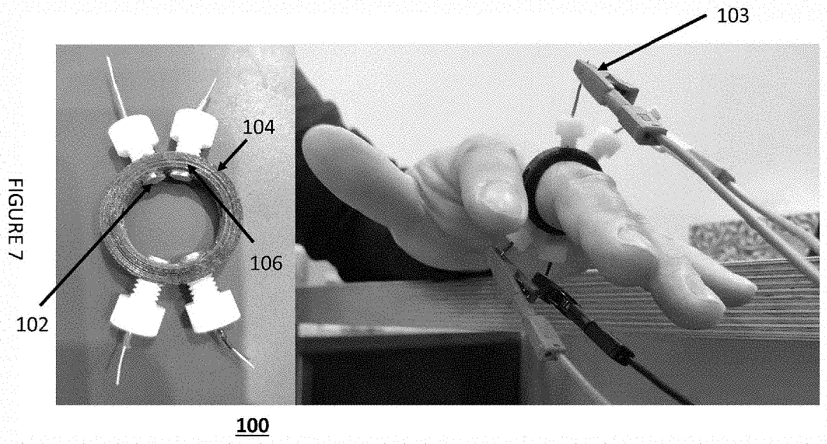

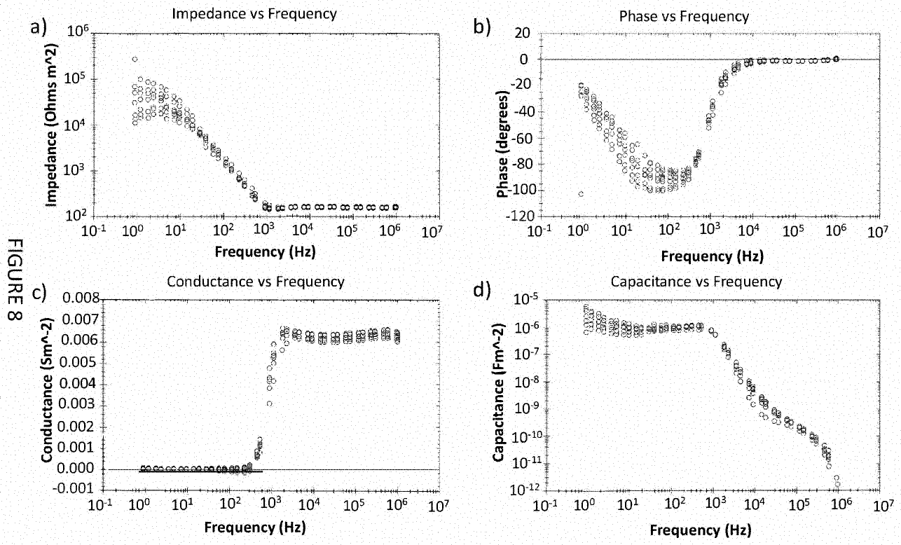

An alternative embodiment of the non-invasive device in the form of a wearable ring having a 4T (four electrode) ring configuration is shown in FIG. 7 and its EIS performance of 7 repeat runs is shown in FIG. 8.

For convenience, the numbering of FIG. 7 showing an alternative configuration has been maintained as per FIG. 5.

Referring to FIG. 7, the non-invasive device 100 in the form of a ring comprises four electrodes 102 which are gold plated for contacting skin. The electrode 102 is adapted to be connected to a receiver (not shown) via alligator clips 103 to process the impedance signals. A housing 104 in the form of a ring has four apertures 106 to receive the four electrodes 102. The electrodes are configured and substantially opposed to each other around the internal periphery of the ring 100 such that in use an electrical current passes through a portion of a subject (i.e., a finger) in use.

In this embodiment, two electrodes are current injecting (stimulating) electrodes and two electrodes are voltage measurement (sensing) electrodes to measure impedance.

In use, the electrodes 102 are powered by the external receiver which is an EIS instrument.

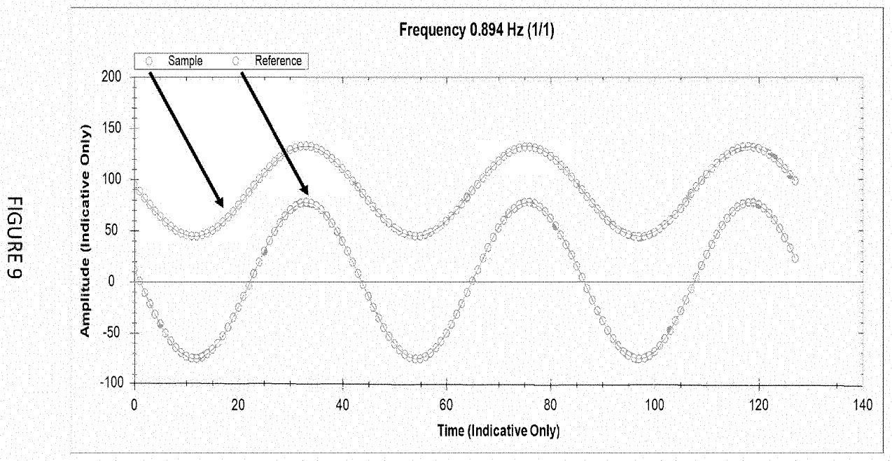

The embodiment of FIG. 7 showed good measurement signal quality, as shown in FIG. 9.

Bracelet

Design

A non-invasive device in the form of a wearable bracelet was also developed as an alternative embodiment, measuring signals through the wrist of a subject. A ‘clamp’ bracelet design was rigid and allowed for fixed positioning of electrodes on either side of a wrist for high quality signals.

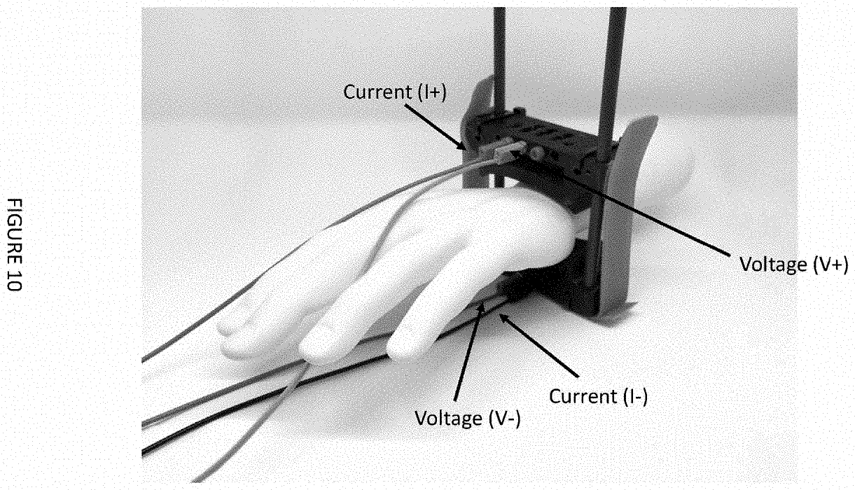

The bracelet design allowed for the largest electrode-skin contact area. Larger electrodes were able to be integrated into the bracelet. Notably, unlike the ring or a watch design, the electrodes in this embodiment are fixed to the housing and cannot be removed without disassembling the electrode for one embodiment of the invention. The bracelet embodiment is shown in FIG. 10.

Manufacturing and Assembly

Similar to the ring, the bracelet is initially printed on the Ultimaker 3 3D printer. Depending on the model, either there are pre-made apertures for the electrodes, connectors, supports and Velcro® (hook and loop fastener), or there are only pre-made apertures for the supports and the remaining apertures can be drilled manually.

After the bracelet has the appropriate cuts, banana connectors soldered to a copper wire are inserted into the housing. The loose end of the copper wire is threaded through and joined to a solid gold piece that acts as the electrode. Adhesive is then applied to the gold piece and it is joined to the housing. The supports are subsequently added with the Velcro® and after the adhesive has dried the bracelet is ready.

EIS Performance

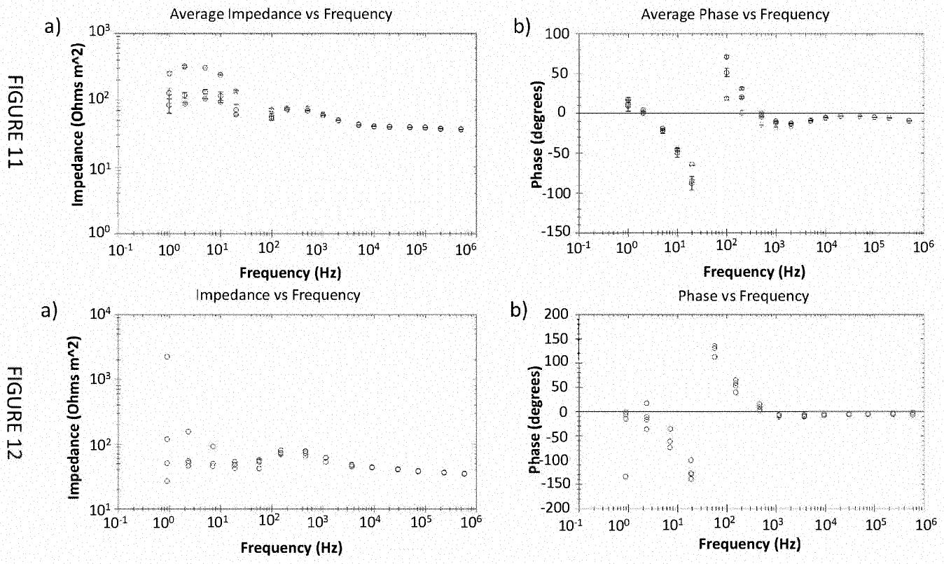

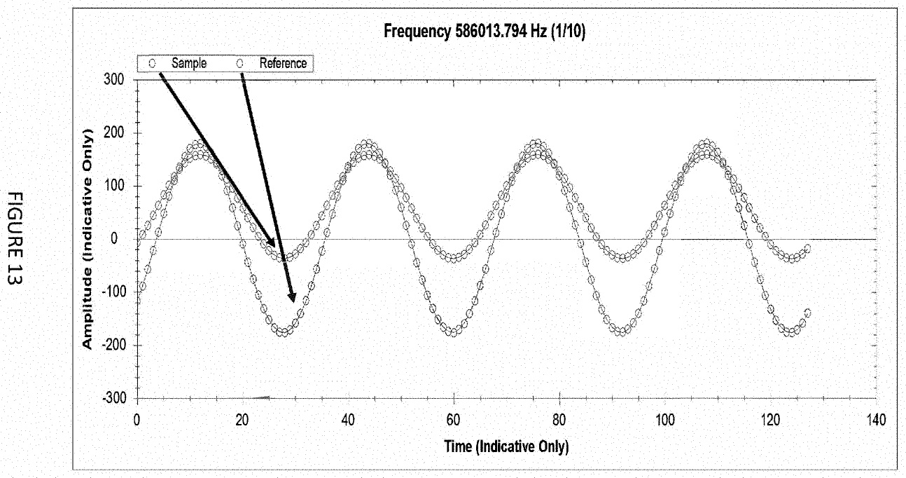

The result of the 4T bracelet measurement (4 repeat runs) using an EIS system is shown in FIG. 11. FIG. 12 shows the same measurement but taken by the Inphaze system (4 repeat runs) for cross-checking.

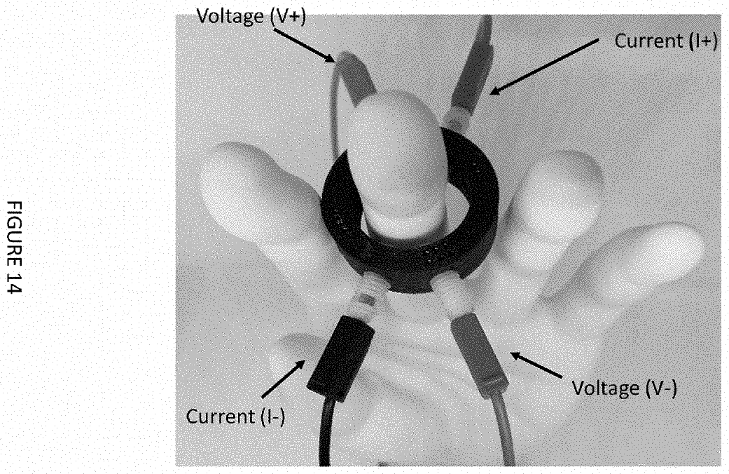

In the bracelet design of FIG. 10, observed with both EIS systems, at around 30-40 Hz there appeared to be an asymptote in the phase profile. The signal quality was good in the measurements, as shown in FIG. 13.

Watch

Design

A non-invasive device in the form of a watch was also developed as an alternative embodiment, measuring signals through the wrist of a subject. The watch provides an adjustable strap.

The watch and bracelet share many similarities, however there are a few notable differences. The watch was designed for usage on only one side of the wrist, whereas the bracelet has the ability for both. The watch was designed for the removable electrodes, whereas the bracelet with fixed electrodes. As smart-watches are prevalent on the market, the pathway for integration to a smartwatch is clear. Additionally, the watch has a snug-fitting adjustable strap that is convenient to adjust and remove.

In respect to the watch itself, there are 8 apertures for removable electrodes that would be compatible with the ring. These 8 apertures enabled the potential for different electrode configurations. Near-flush electrodes were desired as to not protrude too far into the wrist, thus inserts were made for electrodes that did not screw into the body of the watch. In addition to the electrodes, watch straps from an existing watch can be inserted to hold the watch in place. In later designs, inserts for a thermocouple and another small sensor to measure of physiological parameters were incorporated.

Manufacturing and Assembly

The watch is convenient to manufacture and assemble. After the housing is 3D printed, the straps can be added and the relevant electrodes can be inserted.

Conclusion

Various electrode configurations and electrode materials had been developed and tested for the non-invasive device. Several versions of ring, bracelet and watches have been developed, typically with a 4-terminal (4T) electrode configuration to maximise measurement sensitivity.

It was found that the ring with pure gold electrodes performed well in measuring the bioimpedance through the finger. It was comfortable to wear and was able to be worn repeatably at the same location each time.

### EXAMPLE 5

### Electrical Impedance Spectroscopy (EIS) System Testing on Human Subjects

This example outlines some hard and soft requirements for measurements on human subjects for clinical trials. The trial was a protocol-demanding, labour-intensive and time-consuming procedure.

Human Trial Protocol and Requirements of Wearable

Typically, during the human clinical trial, every 10 minutes the test participant would undertake a burst of 10 back to back measurements. This included (not in order) multiple blood glucose concentration measurements, temperature and pH measurements, heart rate and blood pressure measurements and 3 types of bioimpedance measurements. The 3 types of bioimpedance measurements were:
ImpediMed full body measurement using ImpediMed SFB7 and ImpediMed gel electrodes;
4-Terminal wrist measurement using ImpediMed SFB7 and ImpediMed gel electrodes; and
4-Terminal finger measurement using an EIS instrument and ring.

The requirements of the non-invasive device to be used for the context of the trial were:
1. Measurement time: ideally under 1 minute due to the intense measurement protocol;
2. Signal quality: better the measurement signal quality, the more reliable the data;
3. Repeatability: under the same condition, how well can the result be reproduced;
4. Ease of use and comfort: due to the intense measurement protocol the non-invasive device should be comfortable to wear and easy to connect/operate for extended periods of time; and
5. Distinguishability of EIS data for different blood glucose concentrations.

In order for the EIS instrument to achieve the targeted measurement time of 1 minute, one could either decrease the number of frequencies in the scan, especially the low frequencies (<10 Hz), or reduce the number of spectra. In most commercial EIS instruments, usually only 1 spectrum is measured. To be statistically sound, at least 3 spectra should be taken—that is, each frequency should be measured 3 times. After fine-tuning the EIS system, the measurement time was reduced to a satisfactory 63 seconds (5 Hz-500 KHz, 3 spectra).

Performance of Prototype Wearables

The data acquisition software of the EIS instrument featured a unique tool in the form of a “soft oscilloscope” where the acquired discrete data points were plotted against their respective theoretical waveforms (continuous sinusoidal). Good measurement signal quality meant the discrete points fell right onto their respective theoretical curves. While the soft oscilloscope provided instant visual representation of the signal quality, noise (mV) provides numerical information on the signal quality.

Use of these parameters provides a point of reference for evaluating the performance of the non-invasive device of the present invention according to the requirements described above.

Ring

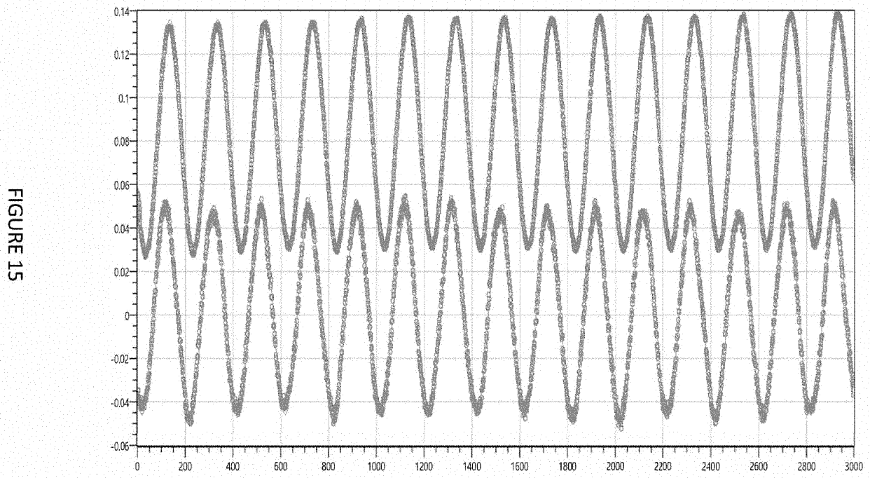

Several ring designs and electrode configurations had been developed and tested in order to obtain the optimum performance for bioimpedance measurement through the finger. The chosen design for further investigation is shown in FIG. 14.

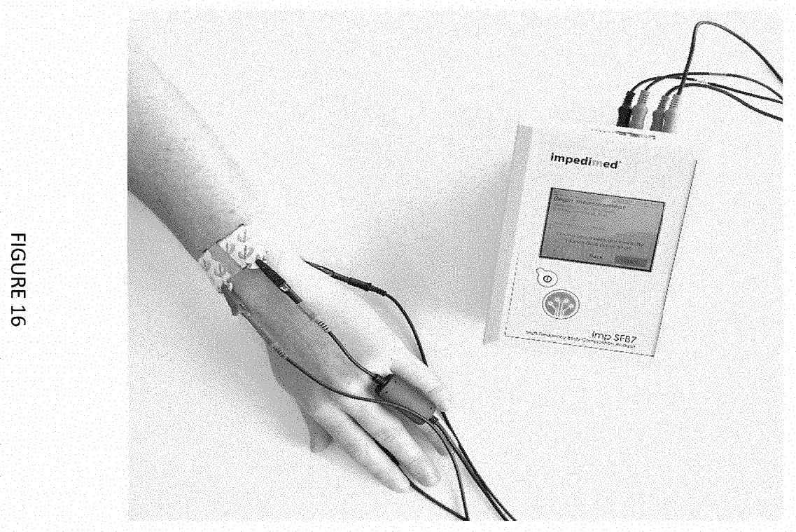

In another embodiment, a 6-Terminal ring configuration was also investigated. The 6 terminals were i+, v+, i+ on the top side and i−, v−, i− on the bottom side, where i=current and v=voltage. This design had extra current injecting electrodes but did not yield any noticeable improvement in signal quality and repeatability which indicated that the distribution of the current field was already sufficient without the extra pair, therefore a 4T configuration was sufficient for human trials as it is physically more convenient to use and operate. FIG. 15 shows the EIS performance of the optimised 4T ring that was used for the human trial.

To ensure consistency in the trials, before each measurement the electrodes of the ring were cleaned with isopropyl alcohol (isopropanol) and the finger was shaven and also cleaned with individually packed skin alcohol wipes.

Bracelet

Bracelet for bioimpedance measurements on a wrist

FIG. 10 shows a photograph of an embodiment of the non-invasive device in the form of a bracelet where the current injecting electrodes were 5 mm×20 mm pure gold strips, 1 mm thick, and the voltage sensing electrodes were 5 mm×5 mm pure gold squares, 1 mm thick.

As with the ring, a 6-Terminal bracelet configuration had also been developed. The 6 terminals were i+, v+, i+ on the top side and i−, v−, i− on the bottom side, where i=current and v=voltage.

Gel Electrodes for Bioimpedance Measurements on Wrist

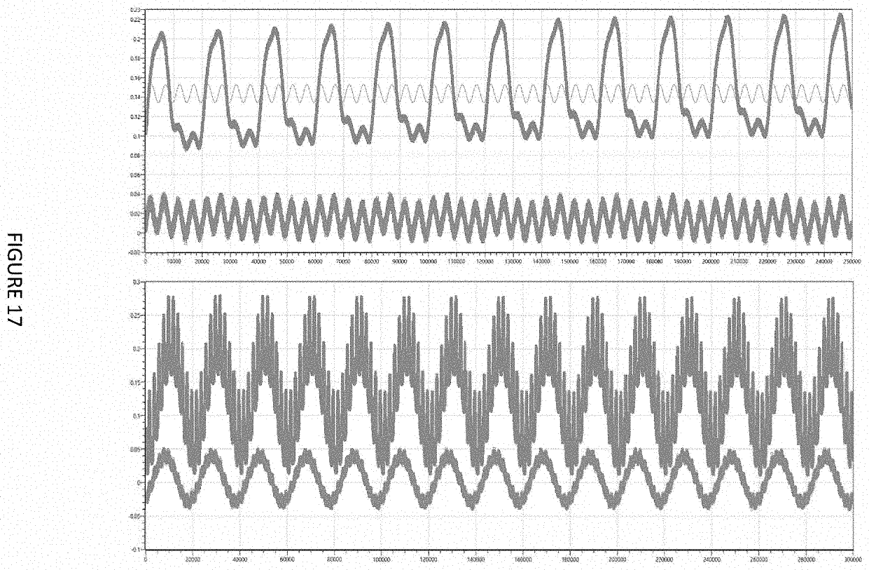

Gel electrodes from ImpediMed were also used for measuring the impedance on a wrist during the trials. The configuration is shown in FIG. 16. The ImpediMed SFB7 as a general-purpose EIS instrument was used, not its intended full body setup, thus the body composition calculations such as FFM, TBW etc were discarded, only the EIS raw data will be kept and used.

Electrical Interference

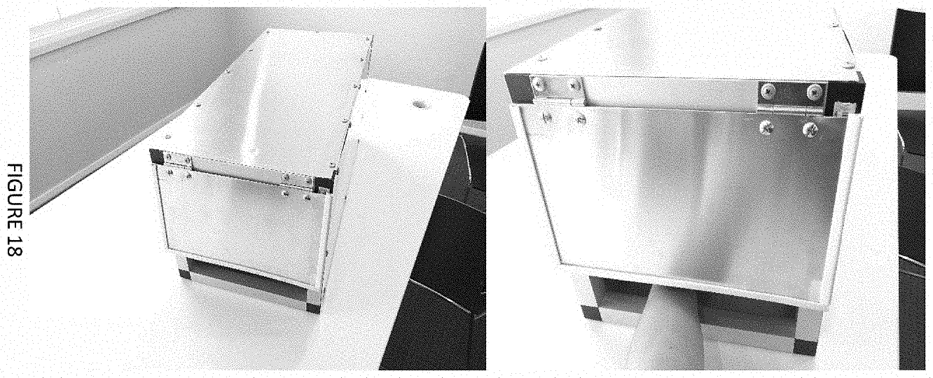

During system testing, electrical interferences can be seen in certain embodiments such as the “soft oscilloscope”. After a series of systematic experiments, it was found that the source of interference came from extension power boards and power adapters for computers and monitors. The impacts of the interferences are shown in FIG. 17. It was clear that the signal data points did not follow their theoretical sinusoid waveforms and the changes real-time can be observed by moving the power cords closer to and then away from the measurement or subject.

To prevent or ameliorate interference, the power cords and laptop chargers can be repositioned away from the measurement during trials.

Faraday Cage

Design and Assembly

The Faraday cage was in one embodiment was a metal box that was large enough to accommodate the EIS instrument and also for a portion of the subject such as a forearm to fit to make bioimpedance measurements of the finger/wrist inside the cage. All sides of the Faraday cage should be well shorted together (electrically), including the door. The cage had a simple small opening at the back for USB and power cables to pass through.

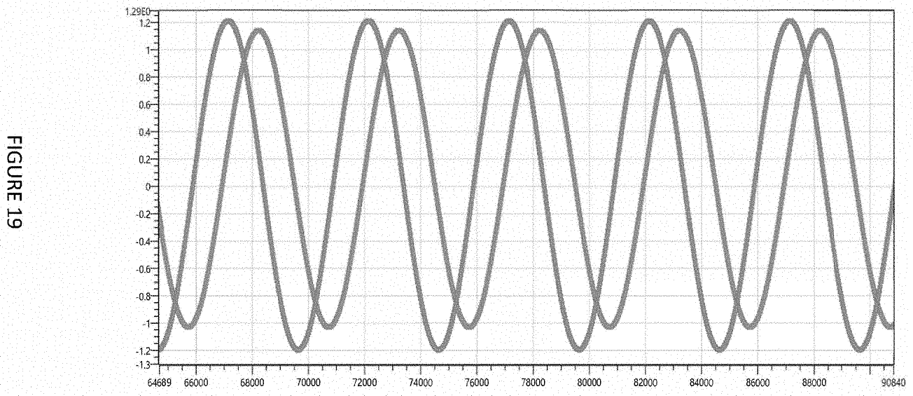

The frame was constructed using aluminium bars while sides and door were constructed using aluminium sheets. Metal screws and metal butterfly hinges were used to bolt the pieces together whilst ensuring good electrical contact and conduction. Finally, the Faraday cage had a couple of connection points exposed for connecting itself to the analogue earth of the EIS instrument. The Faraday cage is shown in FIG. 18.

Effectiveness of Faraday Cage

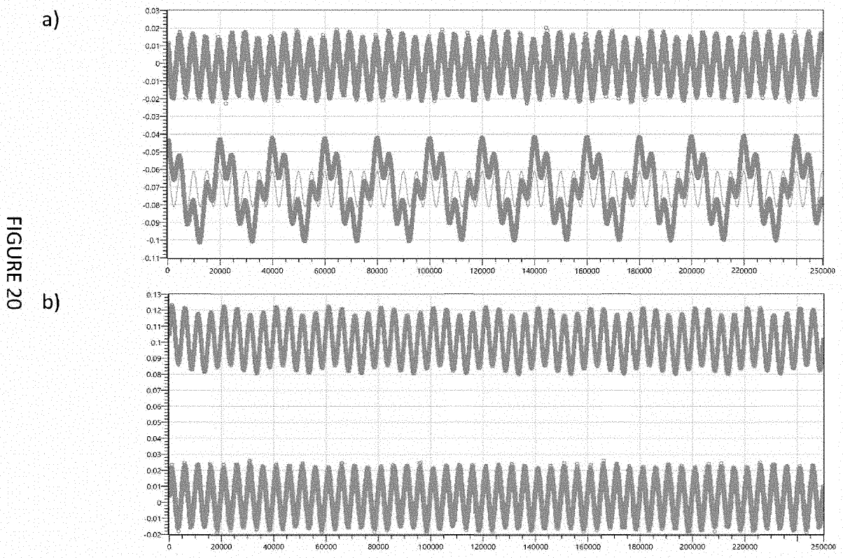

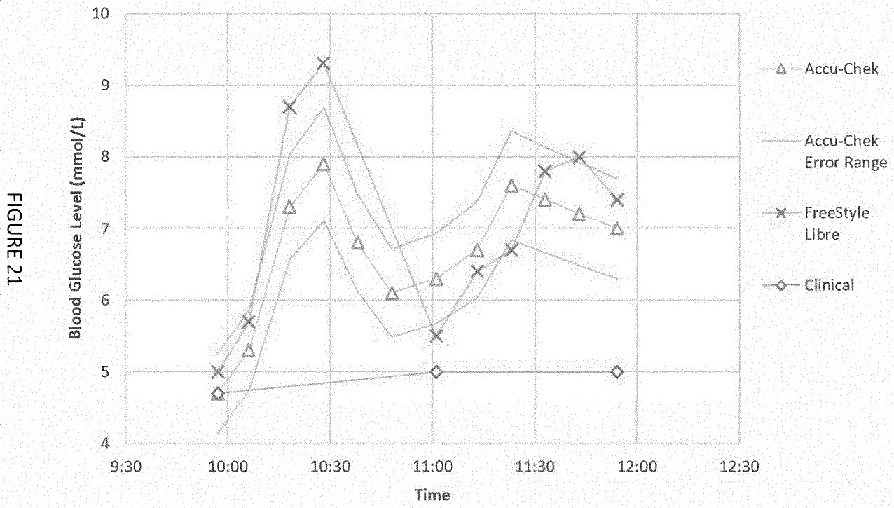

After a series of test runs, it was found the Faraday cage did provide electrical shielding from power cord interferences. When measuring passive components such as resistors and test circuits, the power cord did not have an impact to the signal as shown in FIG. 19. The power cord only had impacts on ring and bracelet measurements and the Faraday cage did provide shielding to a noticeable extent as shown in FIG. 20.

The use of a Faraday cage can prevent or ameliorate signal interferences. However, by carefully positioning all the power cords and computers in a controlled environment, the impact of electrical interferences can be minimised even without a Faraday cage.

Conclusion

According to the protocols followed for the human trial, the requirements of the non-invasive device were:
1. Measurement time;
2. Signal quality;
3. Repeatability;
4. Ease of use and comfort; and
5. Distinguishability of EIS data for different blood glucose concentrations.

A wearable ring was developed and performed satisfactorily for measuring finger impedance during the human trial. Electrical interferences from power cords were also identified and avoided by re-arranging computers and equipment. Robust system testing ensured all components were optimised and the human trial workflow was as smooth as possible.

### EXAMPLE 6

### Human Validation

Intent

A predictive model was developed using a neural network, to predict the blood glucose concentrations of a participant using bioimpedance recorded using electrodes housed in a wearable position. Initially, electrochemical impedance spectroscopy (EIS) devices currently on the market were used as the medical device to record bioimpedance in the configuration defined by the manufacturers. These bioimpedance results were then matched with blood glucose concentration measurements to develop a preliminary predictive model for blood glucose concentration prediction based on bioimpedance results alone.

Participant Selection

Three participants volunteered to be involved in experiments in this study. As per the Australian Code for the Responsible Conduct of Research 2018 and the National Statement on Ethical Conduct in Human Research, ethical review by a human research ethics committee (HREC) is not necessary if the research being undertaken has been determined to be low risk as part of a formal risk assessment procedure. A formal risk assessment was conducted on each process which involved a human participant before any experiments were permitted.

HREC review was determined to not be necessary as each process involved in these human baseline experiments was deemed to be low risk. Each participant involved in this project volunteered to participate and provided verbal consent prior to an experiment being conducted. The validity of this process was confirmed in writing by the National Health and Medical Research Committee (NHMRC) Ethics and Integrity section and the Human Ethics office at the University of Sydney. Considerations were made around the personal data collected in this project. All data collected cannot be published publicly as an HREC review has not been conducted. Limitations were placed on the amount of personal information that was collected and appropriate security measures were in place for privacy reasons. All data in this study was redacted where possible, with each the participants referred to as participant 1, participant 2, or participant 3. A single, secure document was available to match up the participant number with their name if this was necessary. Some relevant information about each these participants is shown in Table 3.

TABLE 3
Participant information about each of the 3 subjects.

| | | | Height | Weight |
|---|---|---|---|---|
| Participant | Age | Diabetic | (cm) | (kg) |
| 1 | 36 | No | 172 | 76.5 |
| 2 | 28 | No | 178 | 70 |
| 3 | 39 | No | 180 | 80 |

Glucose Tolerance Test

Background

Glucose is the body's main energy source. Consumed carbohydrates are broken down into glucose, are absorbed by the small intestine, and are circulated throughout the body. Insulin is produced by the pancreas to control glucose transport into the body's cells or to the liver for storage as glycogen (short-term storage) or to promote synthesis of fats (long-term storage). Insulin is usually released to combat elevated blood glucose concentration after a meal.

Glucagon, another hormone, can be released from the pancreas to release liver glucose stores if blood glucose concentration drops too low. As discussed above, diabetes mellitus is a condition where the body's ability to produce or respond to insulin is impaired, resulting in poorly regulated glucose levels in the blood. Severe and sudden hypoglycaemia (low blood glucose) or hyperglycaemia (high blood glucose) can be life threatening, causing organ failure, brain damage, coma, or death.

Chronic high blood glucose, which can occur with improperly managed diabetes, can cause progressive damage to organs such as the kidneys, eyes, blood vessels, heart, and nerves. Undiagnosed gestational diabetes may lead to babies with a high birth weight, low blood glucose concentration, and nerve or brain damage.

Oral Glucose Tolerance Test (OGTT)

A three-step procedure is used to diagnose type 2 diabetes: (i) initial risk assessment, (ii) measurement of fasting or random glucose levels, and (iii) an oral glucose tolerance test (OGTT). An OGTT is the current gold standard for diabetes diagnosis and is ordered when the results of a fasting or random blood glucose test are equivocal (see Table 4). All pregnant women are tested at 24-48 weeks for gestational diabetes using an OGTT, while women with one or more risk factors (e.g. >40 years of age, familial diabetes history, certain ethnicities) are tested immediately after pregnancy confirmation and again at 24 weeks.

TABLE 4
Fasting or random blood glucose
concentrations required to categorise
a participant as non-diabetic,
requiring an OGTT, or diabetic.

| | Fasting | Random |
|---|---|---|
| | Blood | Blood |
| | Glucose | Glucose |
| Clinical | Level | Level |
| Outcome | (mmol/L) | (mmol/L) |
| Non-diabetic | <5.5 | <5.5 |
| OGTT required | 5.5-6.9 | 5.5-11.0 |
| Diabetic | ≥7.0 | ≥11.1 |

OGTT Procedure

The OGTT participant should consume a regular diet for 3 days and then fast for 8 hours immediately before the test. Only water can be consumed during this fasting period. Smoking is not allowed nor consumption of caffeinated drinks, and medications must be noted as some (e.g. corticosteroids, beta-blockers, diuretics, and antidepressants) can interfere with the test results. A blood test (via venesection) is performed after fasting to record the participant's baseline (or fasting) blood glucose concentration. A glucose drink, manufactured by Point of Care Diagnostics in Australia (product #GTT75), containing 75 g of glucose in filtered water, is consumed within 5 minutes. Further blood is taken at 1 hour and 2 hour timepoints. Minimum exercise should be performed during the test and only small volumes of water should be consumed. The blood glucose concentration is recorded in a pathology laboratory using high pressure-liquid chromatography techniques. Results are typically obtained within 2 business days.

OGTT Principles

Blood glucose concentration reflects the balance between carbohydrate absorbed from the gut, hepatic glucose uptake or output, and peripheral (largely muscle) glucose uptake. Following fasting, an OGTT participant baseline blood glucose concentration represents the hepatic glucose output.

Assuming the participant rests during the OGTT, the blood glucose concentration at 1 hour and 2 hour post-drink consumption represents the combination of glucose load and any hepatic glucose output during the test. Fasting and 1 hour and 2 hour post-drink consumption blood glucose concentration associated with the onset of specific microvascular complications (retinopathy, nephropathy, and neuropathy) and macrovascular complications (atherosclerotic vascular disease) of diabetes have been identified and these values are used as the diagnostics levels for the absence or presence of diabetes.

OGTT Results

Diabetes is diagnosed if the fasting and/or 2 hour post-drink consumption blood glucose levels exceed 7.0 or 11.1 mmol/L, respectively, in the presence of symptoms typical of diabetes (see Table 5). In the absence of symptoms, a second abnormal blood test on a separate day is required. The criteria for gestational diabetes diagnosis for fasting and 2 hour post-drink consumption blood glucose concentrations are 5.5-6.9 and 8.0-11.0 mmol/L, respectively.

TABLE 5
Fasting or 2-hour post-drink consumption blood
glucose concentrations required to categorise
a participant as non-diabetic, prediabetic,
or diabetic in an OGTT

| | | 2 h Post- | |
|---|---|---|---|
| | | Glucose | |
| | Fasting | Challenge | |
| | Blood | Blood | |
| | Glucose | Glucose | |
| Clinical | Level | Level | |
| Outcome | (mmol/L) | (mmol/L) | Implications |
| Non- | ≤6.0 | <7.8 | No excess micro- |
| diabetic | | | nor macro- |
| | | | vascular risk |
| Pre- | 6.1-6.9 | 7.8-11.0 | Excess macro- |
| diabetic | | | but not micro- |
| | | | vascular risk |
| Diabetic | ≥7.0 | ≥11.1 | Excess macro- and |
| | | | micro-vascular risk |

The OGTT will not differentiate between the type of diabetes, predict responses to hypoglycaemic therapy, or indicate current or future risks of diabetes complications. Although the test is the gold standard, it is sensitive to incorrect participant preparation, test administration, and intra-individual variability. Repeating an OGTT may be considered if the results are marginally abnormal and there are potential influences of incorrect participant preparation or test administration.

Glycated Haemoglobin (HbA1c)

Diabetes can also be diagnosed by measuring glycated haemoglobin (HbA1c) levels in human blood. This measurement is standard with modern OGTTs in Australia. HbA1c is a form of haemoglobin covalently linked to a glucose molecule. The HbA1c levels in blood reflect the average blood glucose concentrations over the previous 8-12 weeks rather than at a specific timepoint, with increased levels consistent with prolonged increased blood glucose concentrations.

HbA1c levels can therefore be measured at any time, even if a participant is not in a fasting state. HbA1c testing is the preferred method for assessing glycaemic control in diabetics. The utility and convenience of the test is balanced by the limited availability in many countries, poor standardisation, and higher relative cost. The accepted threshold for diabetes diagnosis is ≥6.5% (or ≥48 mmol/mol), with a repeat test used to confirm diagnosis in the absence of clinical diabetes symptoms and elevated blood glucose concentration. HbA1c levels in the 5.7-6.4% range are deemed high risk.

Clinical Oral Glucose Tolerance Test Results

Clinical OGTTs were performed on participant 1 and participant 2 throughout this study as a method to understand their responses to glucose challenge over time and to compare the accuracy of blood glucose concentration measurements made using an Accu-Chek device to the clinical blood glucose concentration results.

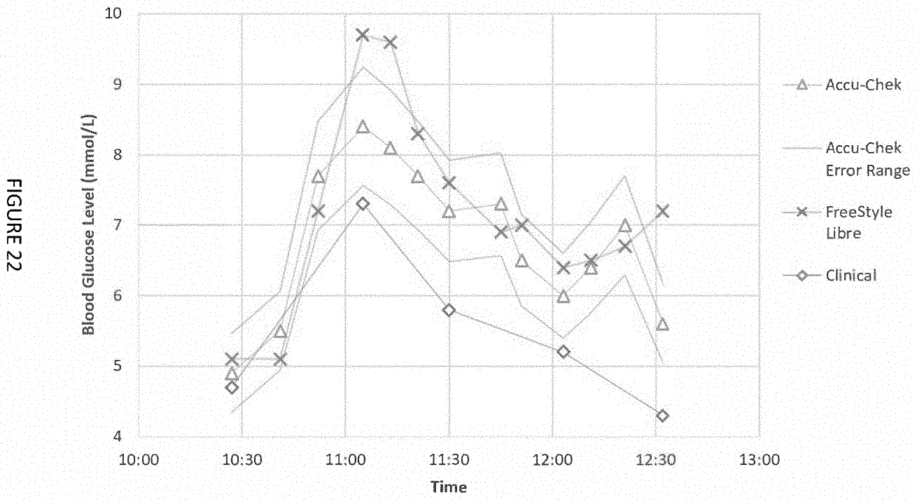

The clinical OGTTs were ordered through iMedical (an online platform that enables private, customisable blood tests) and conducted at Laverty pathology centres. Limited information was gleaned from the first glucose tolerance test performed on participant 1 as the routine OGTT testing procedure only involves blood tests at 1 hour and 2 hour timepoints while the initial blood glucose concentration rise and fall as measured using an Accu-Chek device was within the 0-1 hour period as shown in FIG. 21.

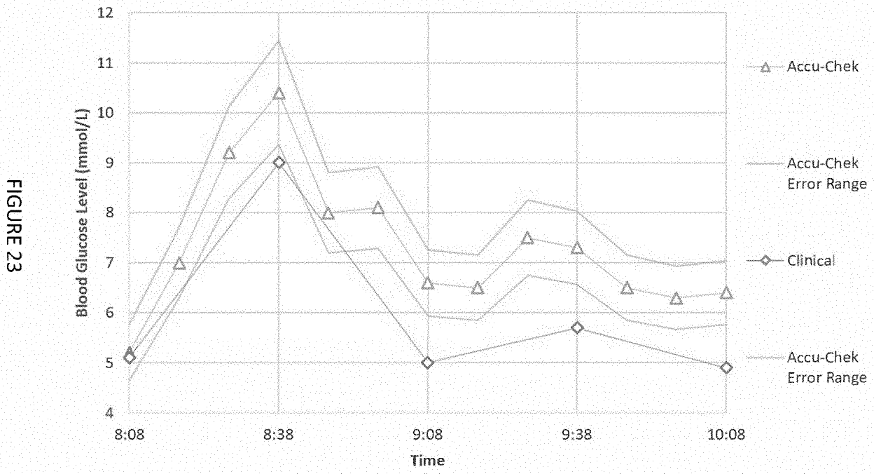

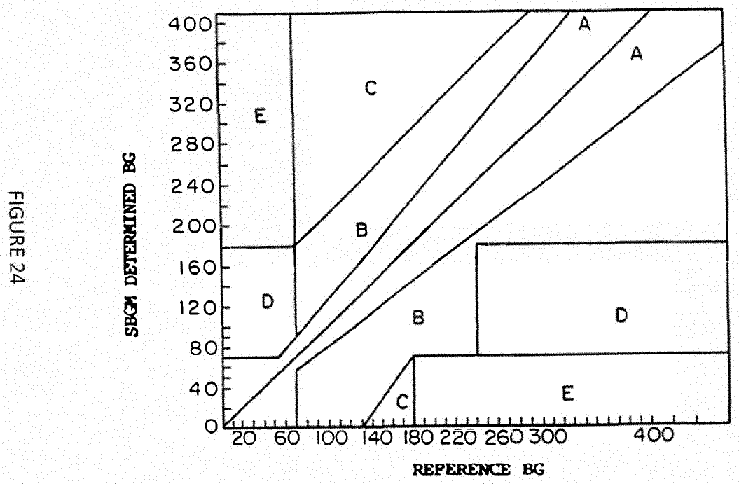

A repeat clinical OGTT was thus performed on participant 1 and was modified to incorporate blood tests every 30 min during the 2 hour testing procedure as shown in FIG. 22. This same, modified OGTT was also performed on participant 2 as shown in FIG. 23.

In each case, the blood glucose concentration value given by the Accu-Chek device trended higher than the blood glucose concentration value given by the clinical result, often falling outside the error range given by the Accu-Chek. The trend of a sharp blood glucose concentration rise and fall back to roughly fasting levels within the first 1 hour period was consistent among OGTTs.

Dual Energy X-Ray Absorptiometry (DEXA)

Dual energy X-ray absorptiometry (DEXA) is the gold standard method for determining bone mineral density for diagnosis of conditions such as osteoporosis. It is a non-invasive scan that determines the density of bones and other tissues by sending two low dose X-rays into the body which are absorbed differently by bones and soft tissues. DEXA has been commercialised as the gold standard method for determining body composition, providing information on body weight, body fat percentage and location, and muscle mass and location.

A DEXA scan was performed on participant 1 as the gold standard method for determining body composition which could be then compared to the body composition analysis performed using the ImpediMed impedance device. The key result from this DEXA scan that could be used to compare to the ImpediMed impedance result is that the total body fat percentage of participant 1 was calculated at 19.7% as shown in Table 6.

TABLE 6
Body composition analysis of participant
1 as determined by DEXA.

| Body | BMC | Fat | Lean | Lean + | Total | % |
|---|---|---|---|---|---|---|
| Region | (g) | Mass (g) | Mass (g) | BMC (g) | Mass (g) | Fat |
| L Arm | 194.25 | 660.2 | 3196.8 | 3391.0 | 4051.2 | 16.3 |
| R Arm | 196.26 | 667.0 | 3306.2 | 3502.5 | 4169.5 | 16.0 |
| Trunk | 757.40 | 6974.0 | 29481.0 | 30238.4 | 37212.4 | 18.7 |
| L Leg | 462.54 | 2700.6 | 9603.6 | 10066.1 | 12766.7 | 21.2 |
| R Leg | 465.49 | 3003.1 | 9590.2 | 10055.7 | 13058.8 | 23.0 |
| Subtotal | 2075.94 | 14004.9 | 55177.8 | 57253.7 | 71258.6 | 19.7 |
| Head | 503.67 | 1052.9 | 3526.3 | 4029.9 | 5082.9 | 20.7 |
| Total | 2579.61 | 15057.9 | 58704.0 | 61283.7 | 76341.5 | 19.7 |

Blood Glucose Concentration Monitoring

Error Grid Types

Monitoring blood glucose concentration is an essential component of diabetes management, informing treatment decisions to improve the prognosis of people with diabetes. Many different devices exist for monitoring blood glucose concentration, each of which must be validated by multiple metrics before being eligible to be taken to market.

ISO 15197:2013 defines that compared to a reference laboratory method, 95% of the blood glucose results of a device need to be within ±0.8 mmol/L for glucose concentrations less than 5.5 mmol/L or ±15% at glucose concentrations at or above 5.5 mmol/L. In addition to this requirement, 99% of the blood glucose concentration values must be within zones A and B on a Parkes Error Grid (PEG) produced for type I diabetes management. There are 3 main error grid types used to monitor device performance for BGL monitoring, as described below.

Converting mg/dL to mmol/L

Two units exist for quantifying blood glucose concentration: mg/dL and mmol/L. Different parts of the world use different systems. The Australian convention is mmol/L whereas in the USA, the convention is mg/dL. This means that the bulk of diabetes literature is in mg/dL which needs to be converted to mmol/L for use in Australia. The conversion from mg/dL to mmol/L is shown using Equation 2.

$$BGL ( m mol / L ) = \frac{BG L ( mg / dL )}{18} \qquad ( 2 )$$

where BGL is blood glucose level (equivalent to blood glucose concentration).

Clarke Error Grid (CEG)

The Clarke Error Grid (CEG) was produced by 5 experts from the University of Virginia, based on their clinical practice, as the original error grid produced for monitoring device performance. This error grid compares a reference BGL (x-axis) to a BGL determined with a monitoring device (y-axis) to qualify device performance, where each zone in which a datapoint can fall has a defined meaning:

Zone A: represents no effect on clinical action;

Zone B: represents altered clinical action but little or no effect on clinical outcome;

Zone C: represents altered clinical action and likely effects on clinical outcome;

Zone D: represents altered clinical action that could have a significant medical risk; and

Zone E: represents altered clinical action that could have dangerous consequences.

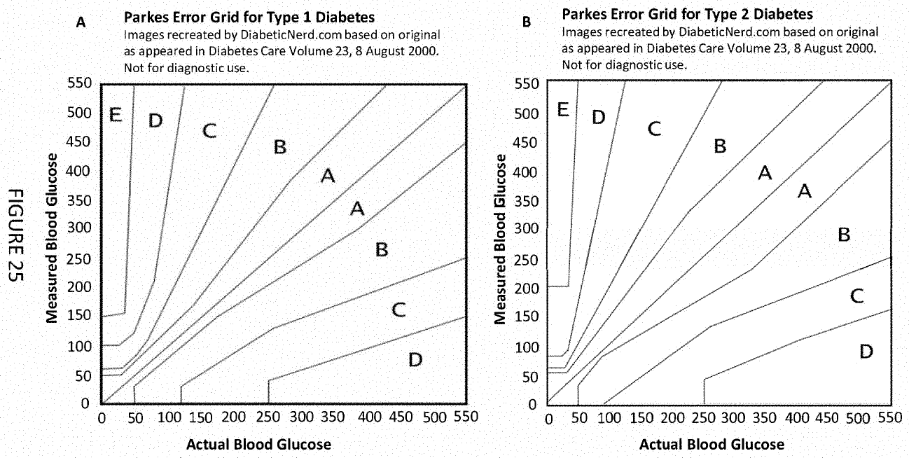

A CEG is shown in FIG. 24.

Parkes Error Grid (PEG)

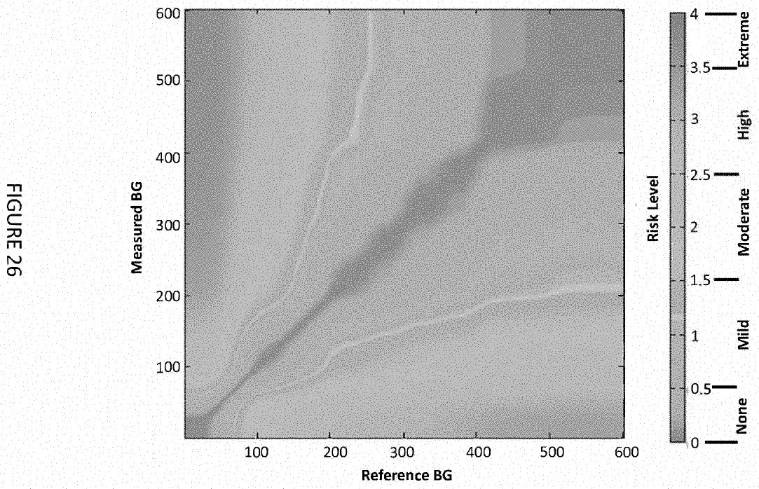

The Parkes Error Grid (PEG), also known as the Consensus Error Grid, was introduced in 2000 to supersede CEG. It was put together by 100 physicians at an annual meeting of the American Diabetes Association to solve some of the previous issues with the CEG: (i) the CEG was introduced by only a small number of experts, (ii) there were discontinuous transitions between zones, and (iii) there was no differentiation between type 1 and type 2 diabetes. Two different PEGs were created, one each for type 1 and for type 2 diabetes with the principle differences being in zones A and B at low blood glucose concentration values as shown in FIG. 25. Despite this, the PEG for type 1 diabetes is most commonly used and is the only error grid covered in ISO 15197:2013. This is the error grid type used throughout this study.

Surveillance Error Grid (SEG)

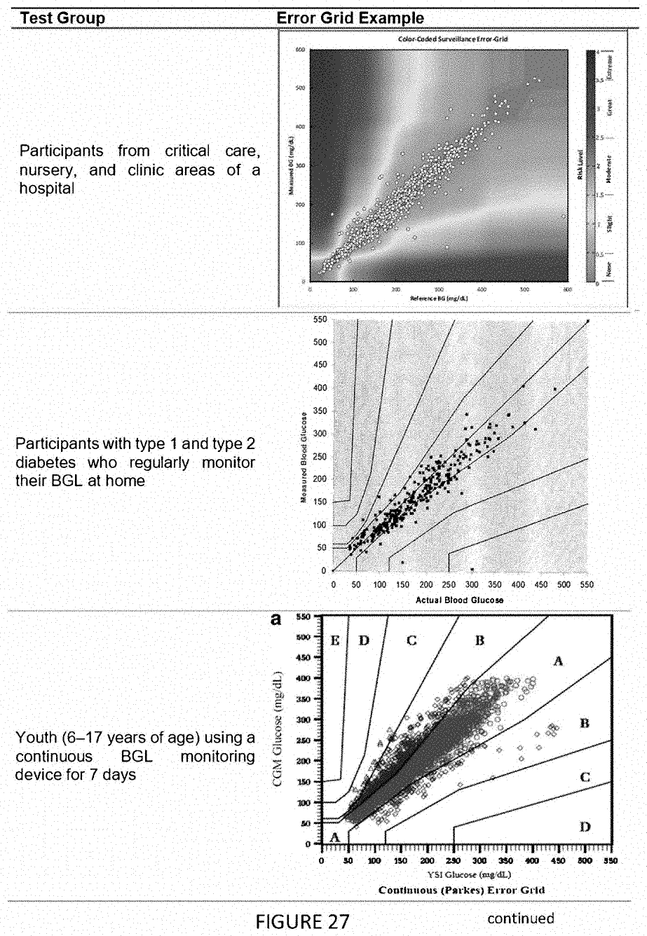

The surveillance error grid (SEG) was introduced in 2014, developed by several authors from academia, industry, and regulatory agencies. It was constructed using a survey by a panel of 206 clinicians and 28 non-clinicians, where each person created their own error grid, and these were then merged. It provides a continuous scale of risk from hypoglycaemia or hyperglycaemia from green (low risk) to red (high risk), with intentions to assist regulatory authorities and manufacturers in assessing the risks from blood glucose concentration monitoring systems that encounter problems in the post-market environment as shown in FIG. 26. Although this is the most recent error grid developed, it does not allow quantification of datapoints within distinct zones and was thus not used in this study.

Blood Glucose Concentration/Level (BGL) Monitoring in Existing Studies

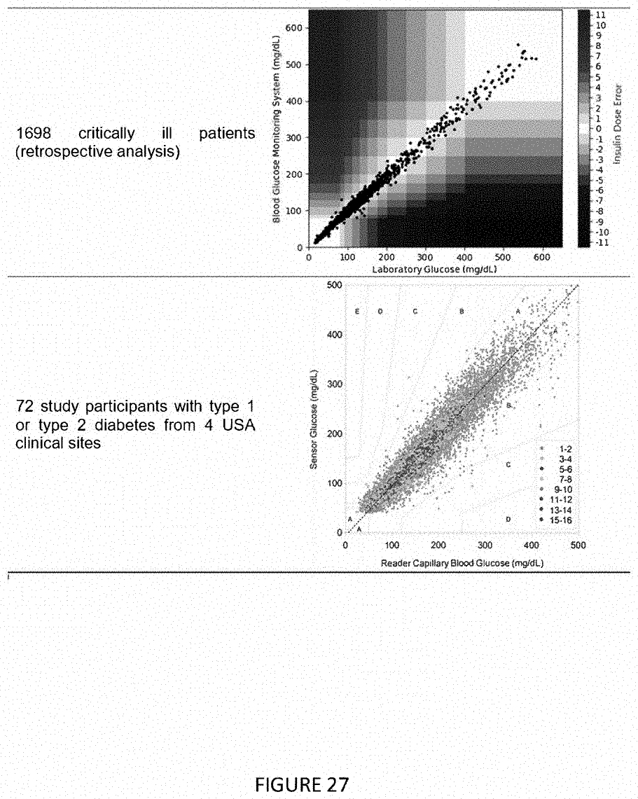

Some published studies were analysed to gain an understanding about the range of BGL values achieved during their testing process with distinct groups of people. In each case, the actual and measured BGL ranged from 0 mg/dL (0 mmol/L) to 500-600 mg/dL (28-33 mmol/L), commonly with a concentration of datapoints in the 50-300 mg/dL (3-17 mmol/L) range. Some representative plots and the participant conditions are included in FIG. 27. Since all 3 participants involved in this project are non-diabetic, the expectation was that a limited range of BGLs would be attained and that BGL manipulation (e.g. using a GTT) would be required to maximise the possible range.

Preliminary Blood Glucose Concentration/Level (BGL) Monitoring in this Study

Initial experiments in this study aimed to provide a sense of the results expected from routine BGL monitoring before BGL measurements were matched up with bioimpedance data to generate the preliminary predictive model. BGL measurements were initially made in isolation on participant 1 with both the Accu-Chek and FreeStyle Libre devices simultaneously over a 4-day period to compare device performance with respect to each other.

This data was then combined with the initial BGL data collected on participant 1 and participant 2 from when BGL and bioimpedance measurements were made simultaneously to broaden the dataset for Accu-Chek and FreeStyle Libre device comparisons.

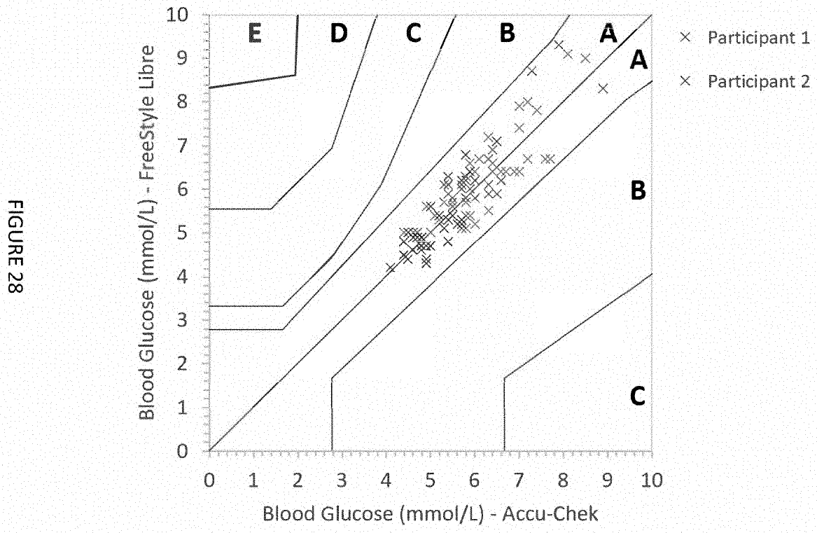

When comparing the BGL results taken at the same time from each of the two devices on a PEG plot, all datapoints fell within the A region as shown in FIG. 28. This demonstrated that each device was performing well with respect to each other, despite the expected time delay with the FreeStyle Libre device.

Preliminary Bioimpedance Results in this Study

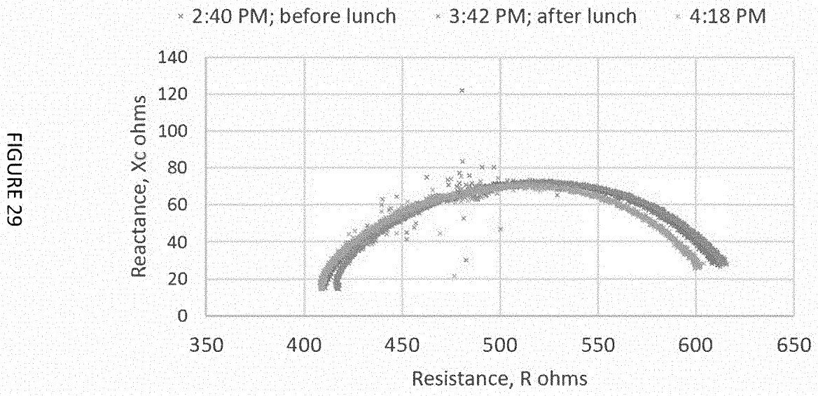

All bioimpedance measurements made as part of the human baseline experiments in this study were made using the ImpediMed impedance device. Within these human baseline measurements, the bulk of the data was collected through the full body using the as per the intended use of the ImpediMed device. The first set of measurements were made to evaluate device consistency. Measurements were made on participant 1 at timepoints surrounding consumption of a meal. Five measurements were made at each timepoint, automated to be taken at 5 second intervals. The reactance and resistance results were consistent within all 5 measurements made at a single timepoint but differed between measurements taken at different timepoints as shown in FIG. 29.

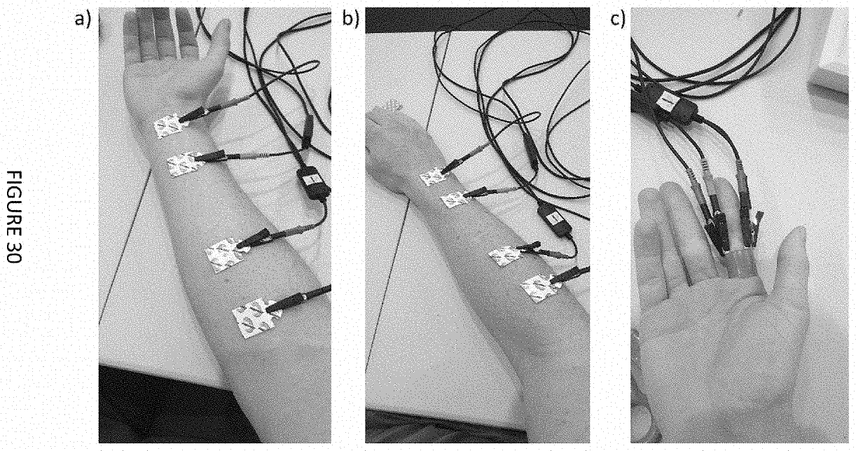

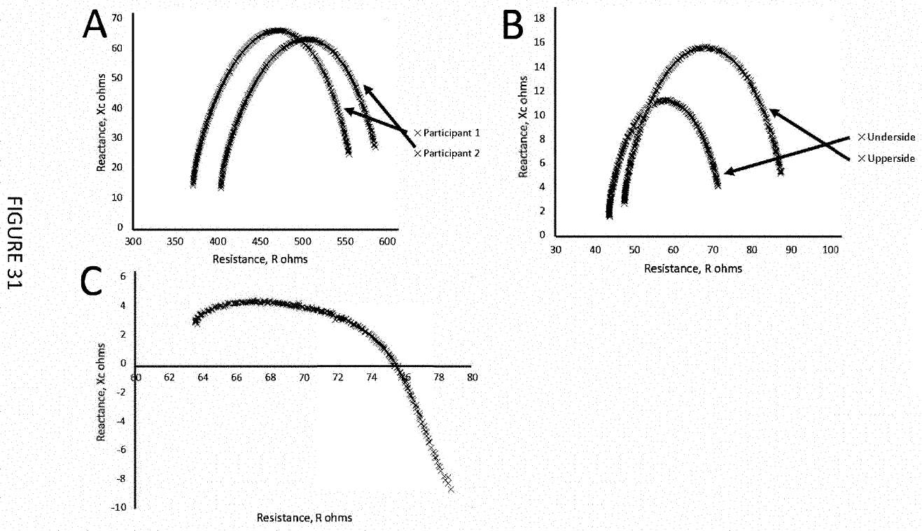

Additional baseline data was collected with the ImpediMed device through the full body, with the ImpediMed gel electrodes attached to the forearm (upper-side and under-side), and with 1 cm strips of the ImpediMed gel electrodes attached to a finger as shown in FIG. 30. In each configuration, the Cole plot (resistance vs reactance) and reactance v frequency and resistance v frequency plots were generated and displayed to provide an example of what typical plots should look like to ensure similar results were obtained during device use as shown in FIG. 31.

Simultaneous Blood Glucose Concentration and Bioimpedance Measurements

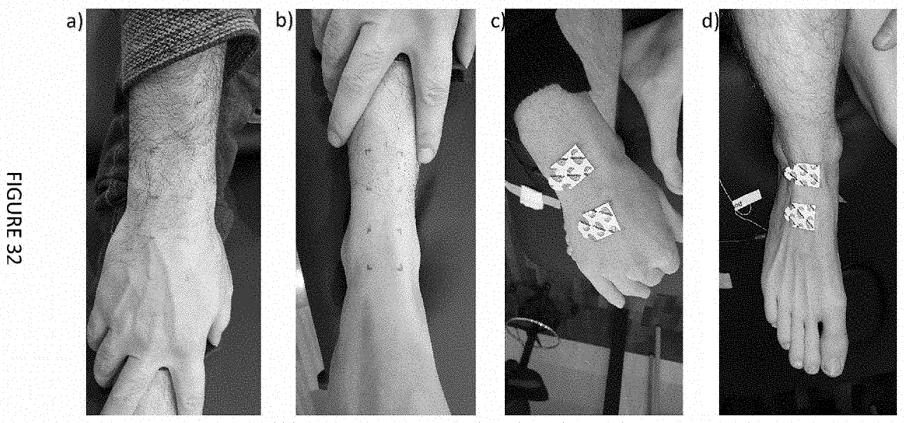

To develop a preliminary predictive model to predict blood glucose concentration based on bioimpedance measurements, bioimpedance data had to be collected in parallel with blood glucose concentration data. Full body bioimpedance was recorded using an ImpediMed device and corresponding gel electrodes placed as per the device operating instructions as shown in FIG. 32.

The site of electrode placement was kept consistent between measurements and new gel electrodes were used for each measurement, even if the measurements were recorded at a narrow timeframe apart from each other. The outline of each electrode was traced with permanent marker to ensure the electrodes were always placed in the same locations. The sites of electrode placement were not shaved prior to placement. For each timepoint, 5 distinct measurements were made with the ImpediMed device at automated 5 second intervals.

Skin temperature was recorded as an ancillary physiological parameter during these bioimpedance measurements. Thermocouples were placed at locations adjacent to electrode placement and the skin temperature was recorded using a ThermaQ device. The thermocouples were placed 2 cm below the bottom electrode on both the hand and feet, towards the fingers or toes. The thermocouple was covered with a folded tissue for insulation.

Blood glucose concentration measurements were made with both the Accu-Chek and FreeStyle Libre devices at times as close as possible to the corresponding bioimpedance measurement. This data was input into a master Excel document alongside the bioimpedance results (both the Cole plot-fitted parameters and the raw data for each of the 5 measurements taken at a single timepoint) and temperature measurements, with each timepoint corresponding to the time at which the Accu-Chek measurement was made. The initial round of data was collected over 7 days on both participant 1 and participant 2. A second round of data collection was completed as a time distinct set of data to be used for independent validation of the preliminary predictive model developed using the results from the first round of data collection. Data was collected from participant 1, participant 2, and participant 3 during these measurements, with the participant 1 and participant 2 data serving as this time distinct set for independent validation and the participant 3 data serving as a test for how well the preliminary predictive model worked on a participant whose data had not been exposed to the neural network.

This data was used as inputs into the preliminary predictive model to enable blood glucose concentration prediction based on the full body bioimpedance results which could then be compared to the actual blood glucose concentration measurements.

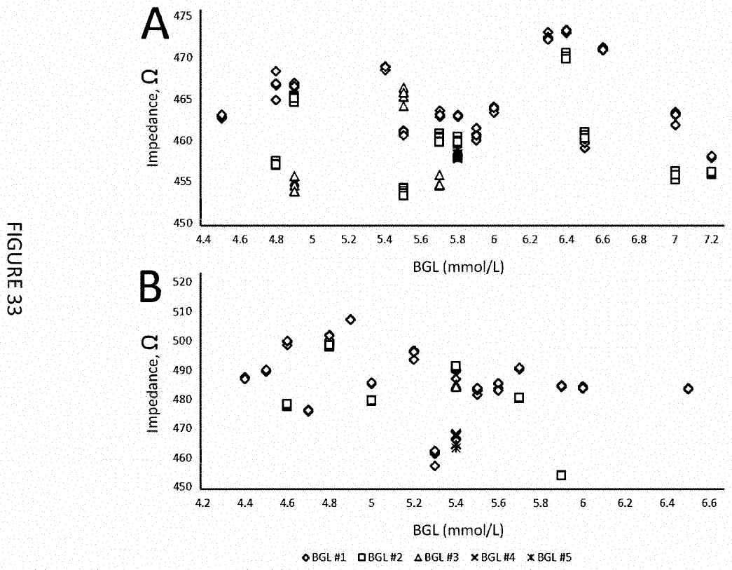

Plots were made for each participant comparing blood glucose concentration (Accu-Chek) to bioimpedance at a range of frequencies over all the data collected in this experiment. No clear trend could be identified to indicate a direct relationship between blood glucose concentration and bioimpedance, which supported the use of a neural network model for the correlation of these inputs. An example plot is shown below for each participant 1 and participant 2 as shown in FIG. 33.

Once all the data had been collected for this dataset, the average values and range for each parameter were determined to inform the range over which these parameters should be expected in future experiments. These were calculated and shown for blood glucose concentration (Accu-Chek, FreeStyle Libre), temperature (hand, feet), body water content, and fat percentage.

### EXAMPLE 7

### Preliminary Predictive Model

Intent

A preliminary predictive model was constructed to determine if it was possible to correlate bioimpedance measurements (BI) with blood glucose concentration/levels (BGL) in study participants from the human baseline study described in Example 6. An Artificial Neural Network modeling approach was selected for this purpose as neural networks are able to identify hidden correlations and were deemed to have sufficiently flexible architecture and parameters for continuous model improvement.

A minimum requirement for the model success criteria was the model being able to predict BGL such that at least 70% of results were in zones A and B based on Parkes Error Grid (PEG) zones. Additionally, it was also desirable that the model be able to track with the BGL trend over time.

Initial modeling work is based on impedance measurements obtained from an ImpediMed® device using as-supplied standard gel electrodes and evaluating through the full body as standard for assessing body composition. The Accu-Chek® SMBG meter as used to obtain pin-prick BGL readings served as reference values for training and testing the model.

The modeling conducted during this phase was work was then used to inform the design of the human trials and the development of the predictive model described in Example 9.

Using R for Data Analysis

The R language was selected for this study due to benefits including open-source licence availability, relatively few cross-platform issues, extensibility via multiple package options, useful supporting documentation, and strong user-community support. The RStudio IDE was used for coding in R.

The “neuralnet” package was used for the neural network modeling and the additional packages employed within the code are described in Table 8.

TABLE 8
R-packages used in model development
obtained from source code

| Package Name | Purpose |
|---|---|
| dataPreparation | for data processing |
| data.table | for handling data tables |
| Dplyr | for data wrangling |
| doBy | utility functions including groupwise |
| | summary statistics |
| Ega | package with Clarke Error Grid functions |
| GGally | for regression modelling |
| lubridate | to work with dates |
| Plyr | for data functions |
| readr | to import csv files |

Source Data

Description of Data Used for Modeling

Bioimpedance data was obtained from the ImpediMed® device includes raw values and subsequently processed fitted-parameters. Further, the following ancillary data was measured during the testing:
Participant Height;
Participant Weight;
Participant Age; and
Participant Gender.

Bioimpedance data available from the ImpediMed® device are as follows:
Raw data of Reactance and Resistance at 256 frequencies:
   Frequency (kHz), also referred-to as F in the raw data;
   Resistance (Ohms), also referred-to as R in the raw data; and
   Reactance (Ohms), also referred-to as X in the raw data.
Device-processed data resulting in fitted parameters:
   Frequency (kHz), also referred-to as F in the raw data;
   Cole fit centre X, ohms;
   Cole fit centre R, ohms;
   Cole circle radius, ohms;
   SEE of radius, % R (zero), ohms;
   R (infinity), ohms;
   Re, ohms;
   Ri, ohms;
   Z characteristic, ohms;
   f characteristic, kHz; and
   Membrane capacitance, nF.

The human baseline data used was collected over a period of 2 weeks. Data was collected for the Full Body configuration. It is to be noted that during coding in this initial stage, there was a need to remove the Patient Height, Patient Weight, Patient Age, and Body Mass Index columns from the datasets because of scaling errors when attempting to use the Subject 3 dataset for Testing. This scaling issue was due to scaling the Training and Testing datasets separately, and later circumvented by the use of a Standardisation scaling approach (discussed subsequently).

There were 250 rows of data in total, with 5 impedance results associated with every BGL data point obtained due to repetitions during data collection. The dataset made use of the device-processed data fitted parameters described above.

Data Scaling

The dataset needs to be numerically scaled to work well with the neural network package due to the algorithms used. In preliminary modeling, the scaling method is as follows:
1. Maximum and minimum values of dataset are identified;
2. Data range calculated as difference between maximum and minimum values identified above; and
3. Dataframe scaled with minimum values set as center using the range calculated above.

Two improved methods of scaling (Normalisation and Standardisation) were explored during subsequent stages of model development, as described later in Example 9.

Neural Network Design

Description of Model and Architecture

A two-layer neural network model with 3 nodes per layer was used in the neural network model. Two hidden layers were used to allow for greater model flexibility and increase likelihood of finding correlations in the data compared to using one hidden layer. Two layers were used as the upper limit to avoid overfitting. Three nodes per layer were selected for initial testing and these were later varied during the phase to evaluate model performance.

Activation Function

The activation function is a differentiable function used for smoothing the result of the cross product of the covariate or neurons and the weights. By default, the linear activation function is used (“linear.output=TRUE” in neuralnet code), however hyperbolic tangent and logistic functions can also be used.

Training and Testing

The full data set was split approximately 80:20 between training and testing sets, and the data points were selected at random. There was no assessment of the distribution of the data to ensure the test set was representative of the model at this stage. For example, we did not assess the number of test values used for each participant.

The model training employs a modified K-fold cross-validation technique to improve the robustness of the model. A 5-fold cross-validation was conducted where the model was trained and validated five times. For each round of training, the training set was randomly split 60-40 between training and validation. That is 60% of the data used for training, 40% for validation, with a different set of data for each round of training.

This is slightly different to a standard K-fold implementation which, for a 5-fold validation is split 80:20 with the 20% systematically chosen such that at the conclusion of training all datapoints have been used for both training and validation. The present method used does not ensure this—but increases the validation set to minimise the amount of data that might not be used for validation.

Randomness within the model was seeded to ensure reproducibility of results between test runs and also for different computers the testing was conducted on.

Assessing Model Performance

Mean Squared Error (MSE)

The Mean Squared Error (MSE) measures the average squared difference between the predicted and actual values and is calculated as shown in Equation 3.

$$MSE = \frac{\sum {( Predicted Value - Actual Value )}^{2}}{Number of Actual Values} \qquad ( 3 )$$

It is to be noted that the positive sign of the MSE due to the squaring of the numerator removes potentially useful information regarding the skew of predicted results compared to their actual counterparts.

Mean Absolute Relative Difference (MARD)

Mean Absolute Relative Difference (MARD) is an overall measure of accuracy typically used in blood glucose measurement analysis and is commonly referenced when describing the performance of CGMs. It is the average difference between the predicted and actual values, relative to the actual value itself, and is calculated as shown in Equation 4.

$$MARD = \frac{\frac{\sum \vert Predicted Value - Actual Value \vert}{Number of Actual Values}}{Number of Actual Values} \qquad ( 4 )$$

There are a number of limitations and drawbacks with using the MARD as a primary indicator of model performance. It is to be noted that while lower MARDs indicate better fitting than higher MARD values within a dataset, it is difficult to compare MARD values directly between different sets of data due to factors such as spread of data ranges and collection methods.

It is also possible that during comparison, results with a lower MSE may have a higher MARD, requiring additional comparison bases such as the Parkes Error Grid as discussed below.

Parkes Error Grid (PEG) Zones

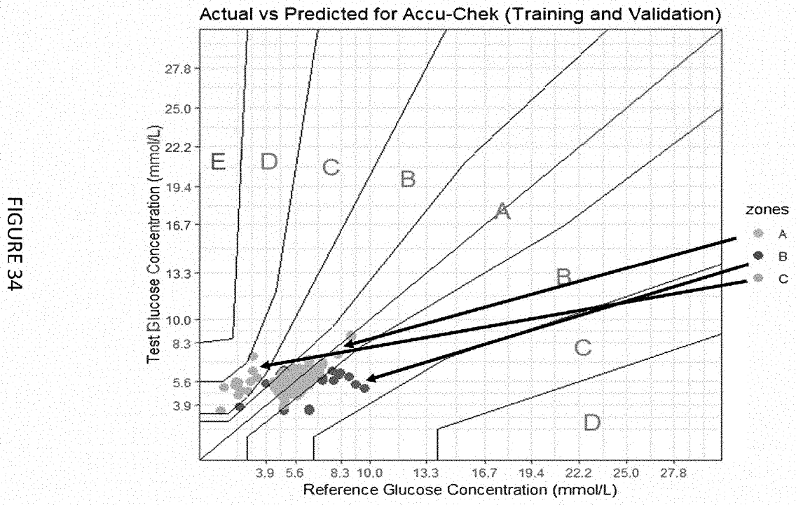

Ideally, the predicted blood glucose concentration values from the model would match near-exactly with relatively small error to the actual blood glucose concentration values obtained from the pin-prick device. The Parkes Error Grid (PEG) is a conventional way of depicting the comparison of predicted versus actual values for blood glucose concentration measurements. Data will fall within one of five zones, A-E based on potential clinical impacts, where zone A is no-impact and zone E has a significant clinical impact. The proportion of points in each zone is quantified during reporting. For example, in FIG. 34, the data points are 89% in A Zone, 7% in B Zone, and 4% in C Zone.

A detailed discussion on the use of error grids for blood glucose concentration predictions is provided under the heading “Error grid types” above.

Model Analysis (Results)

Basic Implementation

The work in this study refers to initial human baseline modeling carried out to assess suitability of using bioimpedance data for modeling blood glucose concentration.

The model uses two hidden layers with three nodes per layer, 5-fold cross-validation and a Training-Validation proportion split of 60-40. Results are summarily tabulated in Table 9. Overall, the model is able to use bioimpedance (fitted parameters) to predict blood glucose concentrations when attempting to model with measurements from the Full Body and Wrist: 100% of PEG Points were located in the A+B zones for both Training/Validation and Testing.

TABLE 9
Implementation of the neural network model
for Sprint 3 data for Full Body.

| | Training + | Training + | | | |
|---|---|---|---|---|---|
| Training + | Validation | Validation | | Testing | Testing |
| Validation | PEG A | PEG B | Testing | PEG | PEG |
| MSE | (%) | (%) | MSE | A (%) | B (%) |
| 0.18 | 100 | 0 | 0.178 | 88 | 12 |

Using Raw Frequency Data for Modeling

The neural network model implementation was modified to predict blood glucose concentration from raw bioimpedance (BI) values from ImpediMed®, specifically:
Frequency (kHz), also referred-to as F in the raw data;
Resistance (Ohms), also referred-to as R in the raw data; and
Reactance (Ohms), also referred-to as X in the raw data.

R and X values between 10-500 kHz were used, in keeping with the frequency range values used by the ImpediMed® when post-processing results for user viewing. Total number of points is 256, 84 rejected outside of the frequency limits, resulting in 172 R and 172 X points used for modeling. Results are shown in Table 10.

TABLE 10
Results from using raw frequency data for modelling.

| Modelling | | | | PEG | |
|---|---|---|---|---|---|
| Stage | Participant(s) | MSE^{a} | MARD^{b} | Zone(s)^{c} | |
| Training | 1 | 0.78 | 7.9 | A | 95.1%, |
| | | | | B | 4.9% |
| Testing | 2 | 1.19 | 18.2 | A | 73.6%, |
| | | | | B | 26.4% |
| | 3 | 0.36 | 8.1 | A | 100% |
| Training | 2 and 3 | 0.30 | 3.6 | A | 99.3%, |
| | | | | B | 0.67% |
| Testing | 1 | 2.60 | 23.1 | A | 61.3%, |
| | | | | B | 36.2%, |
| | | | | C | 2.5% |

^{a}MSE = mean squared error; ^{b}MARD = mean absolute relative difference; and ^{c}PEG = Parkes Error Grid.

Results suggested that the concept of using bioimpedance to predict blood glucose levels within the parameters of this study and dataset continues to be potentially workable when attempting to model with raw output results (viz. F, R, X) instead of device-processed values (e.g. Cole Resistance): majority of points were located within the A and B PEG Zones for all cases studied.

Based on the findings above, a further assessment of predicting variables was attempted, specifically subsetting the frequency points used for Training/Validation and Testing. Results are shown in Table 11.

TABLE 11
Results from using subsets of raw frequency data for modelling

| | Subset | | | | | |
|---|---|---|---|---|---|---|
| Data | Modelling | | | | PEG | |
| Proportion | Stage | Participants(s) | MSE | MARD | Zone(s) | |
| 100% | Training | 1 | 0.78 | 7.9 | A | 95.1% |
| | | | | | B | 4.9% |
| 100% | Testing | 2 and 3 | 1.11 | 17.5 | A | 75.3% |
| | | | | | B | 24.7% |
| 75% | Training | 1 | 0.99 | 9.3 | A | 90.8% |
| | | | | | B | 9.2% |
| 75% | Testing | 2 and 3 | 1.66 | 19.9 | A | 68.7% |
| | | | | | B | 31.3% |
| 50% | Training | 1 | 0.67 | 9.6 | A | 96.3% |
| | | | | | B | 3.7% |
| 50% | Testing | 2 and 3 | 1.54 | 20.0 | A | 66.7% |
| | | | | | B | 33.3% |
| 25% | Training | 1 | 1.17 | 9.7 | A | 93.3% |
| | | | | | B | 5.5% |
| | | | | | C | 1.2% |
| 25% | Testing | 2 and 3 | 2.53 | 26.1 | A | 48.7% |
| | | | | | B | 48% |
| | | | | | C | 3.3% |

The concept of using bioimpedance to predict blood glucose concentration within the parameters of this study and dataset continued to be potentially workable when attempting to model with raw output results (viz. F, R, X) instead of device-processed values (e.g. Cole Resistance): majority of points are within the A and B PEG Zones for the cases studied. Interestingly, utilising less data improved model predictions in some cases, possibly due to outlier values at some frequencies.

Using Subset of Raw Frequency Data: Median Values

Overall, findings suggest that using a median value for each unique combination of date, time, participant, and blood glucose concentration, provides for reasonable prediction of blood glucose concentration values. Training results for the Median have higher MSE, MARD and lower PEG zone accuracies than when using all values without only taking the Median.

On the other hand, testing results when using Median has slightly lower MSE but slightly higher MARD than the former. Further, testing when using Median has slightly higher B zone percentage, slightly lower A zone percentage, and no C zone percentage (whereas not using Median has a small C zone percentage). Results are shown in Table 12.

TABLE 12
Results from using subset of raw
frequency data (median values)

| Modelling | | | | PEG | |
|---|---|---|---|---|---|
| Stage | Participant(s) | MSE | MARD | Zone(s) | |
| Training | 1 | 1.34 | 12.7 | A | 85.7% |
| | | | | B | 14.3% |
| Testing | 2 and 3 | 1.11 | 16.5 | A | 73.3% |
| | | | | B | 26.7% |
| Training | 2 and 3 | 0.67 | 8.8 | A | 93.3% |
| | | | | B | 6.7% |
| Testing | 1 | 2.40 | 22.2 | A | 60% |
| | | | | B | 40% |

Consequently, the approach of obtaining median values within each data subset prior to modeling was deemed potentially non-ideal, and instead utilising all rows of the data obtained was found to be a preferable approach for future modeling.

Assessing Impact of “Noisy” Data

Studies were performed to understand the impact of introducing “noise” into the data by studying the resultant changes to model prediction capability. To this end, 10% of the dataset was modified (i.e. introducing errors) by replacement with random blood glucose concentration values between 1 and 10 mmol/L. Results from this analysis shown in Table 13.

TABLE 13
Results from introducing “noise” into the dataset

| | Modelling | | | | PEG | |
|---|---|---|---|---|---|---|
| Noise | Stage | Participant(s) | MSE | MARD | Zone(s) | |
| 0% | Training and | 1 | 0.41 | 7.1 | A | 97.5% |
| | Validation | | | | B | 2.5% |
| 0% | Testing | 2 | 2.52 | 26.7 | A | 48.8% |
| | | | | | B | 51.2% |
| 0% | | 3 | 1.13 | 15.4 | A | 76% |
| | | | | | B | 24% |
| 10% | Training and | 1 | 1.21 | 13.8 | A | 87% |
| | Validation | | | | B | 11% |
| | | | | | C | 2% |
| 10% | Testing | 2 | 0.71 | 11.4 | A | 91.2% |
| | | | | | B | 8.8% |
| 10% | | 3 | 0.78 | 11.3 | A | 68% |
| | | | | | B | 32% |
| 0% | Training and | 1 and 2 | 0.57 | 8.7 | A | 92% |
| | Validation | | | | B | 8% |
| 0% | Testing | 3 | 0.85 | 13.6 | A | 68% |
| | | | | | B | 32% |
| 10% | Training and | 1 and 2 | 1.27 | 14.9 | A | 89% |
| | Validation | | | | B | 7% |
| | | | | | C | 4% |
| 10% | Testing | 3 | 0.33 | 8.9 | A | 100% |

Overall, the concept of using bioimpedance to predict blood glucose concentration within the parameters of this report and dataset continue to be workable even when introducing errors in the Training/Validation stage. Without noisy data during Training/Validation, 100% of points continue to fall within the A and B zones of PEG plots for both Training/Validation and Testing (using data previously not shown to the model) modeling within the cases tested.

With noisy data during Training/Validation, predictability decreases as expected with some points falling into the C zone of PEG plots, and error values increasing compared to without noisy data. However, both expected and unexpected behaviour was observed for the test cases, with Participant 2 results improving compared to when tested using a model which had been trained on the original data.

It was also found that the MSE and MARD increased for Participant 3 while retaining similar PEG Zone proportions when tested using a model which had been Trained and Validated using data from Participants 1 and 2 compared to when using noisy data from just Participant 1. Accordingly, Training and Validating using one Participant can lead to different degree of blood glucose concentration predictability in other participants.

Conclusion

Overall, the concept of using bioimpedance (BI) to predict blood glucose concentration within the parameters of this study and data collected is found to be workable. It is possible to predict blood glucose concentration using both raw output results (viz. F, R, X) and device-processed values (e.g. Cole Resistance) from the ImpediMed® device, generally with 100% of predictions within the A and B Parkes Error Grid (PEG) Zones for cases reported.

It was found that the neural network model constructed is fit for the intended purpose, exceeding the study success criteria of 70% of predicted points within the A and B zones on a Parkes Error Grid.

### EXAMPLE 8

### Human Testing

Intent

A preliminary predictive model was developed to predict blood glucose concentration/levels (BGLs) as discussed previously based on bioimpedance measurements made across the full body with an ImpediMed SFB7 device and corresponding gel electrodes.

The purpose of human testing experiments in Example 6 was to develop a refined predictive model following learnings from the human baseline experiments and development of the preliminary predictive model. This included incorporating a wider range of ancillary parameters (such as physiological parameters) as inputs into the neural network, to move the locations of electrode placement on the body to places suitable for a non-invasive wearable device (wrist, finger), and to use a EIS instrument for bioimpedance recording.

Methodology

Skin pH Measurement Development

Trials were conducted prior to establishing the final human testing protocol to get a sense of the skin pH of a participant over time and under different conditions. It was found that the pH of different areas on the body (foot, hand, arm) were roughly consistent over a time course extending towards 3-4 h.

The pH measurements on the arm were roughly consistent across different days, while the pH of the hand seemed to fluctuate the most due to different treatments of the hand (e.g. washing) and the foot seemed to fluctuate with sweat production.

Addition of some creams (e.g. moisturiser) had minimal effect, while the addition of others (e.g. sunscreen) had a larger effect. Cleaning the site of pH measurement with an isopropanol wipe led to the most consistent results and was preferred before taking measurements in the human testing protocol.

Wrist Bioimpedance Development

Prior to deciding upon using the ImpediMed with gel electrodes on the wrist, experiments were conducted to confirm that this configuration was viable and produced reproducible results. The gel electrodes were placed in positions below the prominent bone sticking out from the wrist, as placing the electrodes on this bone led to poor results.

Measurements were taken over an extended time course over subsequent days, removing and replacing the electrodes in the same positions over different measurements. These results demonstrated that the acquired data was broadly (visibly) reproducible over each measurement and it was decided that this configuration was suitable for use moving into the human testing.

Final Human Testing Protocol

The human testing protocol was designed to include a broader range of parameters to be measured than with the human baseline experiments. The same 3 participants previously described were involved in these experiments. The parameters measured were:
1. Bioimpedance;
   a. Full body (ImpediMed, gel electrodes);
   b. Wrist (ImpediMed, gel electrodes); and
   c. Finger (EIS instrument, dry electrodes in a ring device).
2. BGL (blood glucose concentration/level);
   a. Accu-Chek; and
   b. FreeStyle Libre.
3. Skin temperature (4 sites);
4. Skin pH (4 sites);
5. Blood pressure (systolic and diastolic); and
6. Heart rate.

Full body bioimpedance was recorded using the ImpediMed device and gel electrodes for the purpose of benchmarking results from this experiment to the preliminary predictive model.

Bioimpedance was recorded across the wrist using the ImpediMed device and gel electrodes for the purpose of transitioning towards a location on the body viable for a non-invasive wearable device but using an EIS instrument and electrode system demonstrated to be amenable to BGL prediction using a neural network model as shown in FIG. 16.

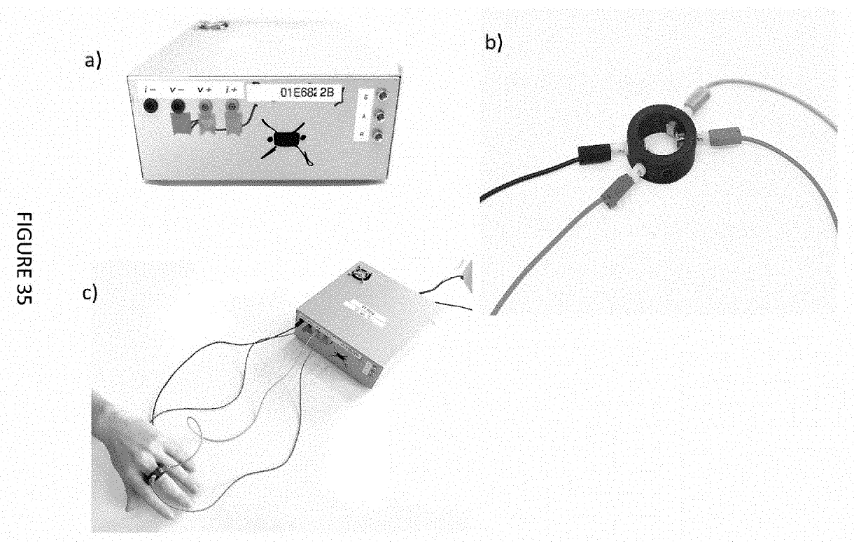

Bioimpedance was recorded across the finger using an EIS instrument and prototype dry electrodes for the purposes of moving towards a location on the body viable for a wearable device and using a prototype EIS device and dry electrode combination that could be adapted into a non-invasive wearable device as shown in FIG. 35.

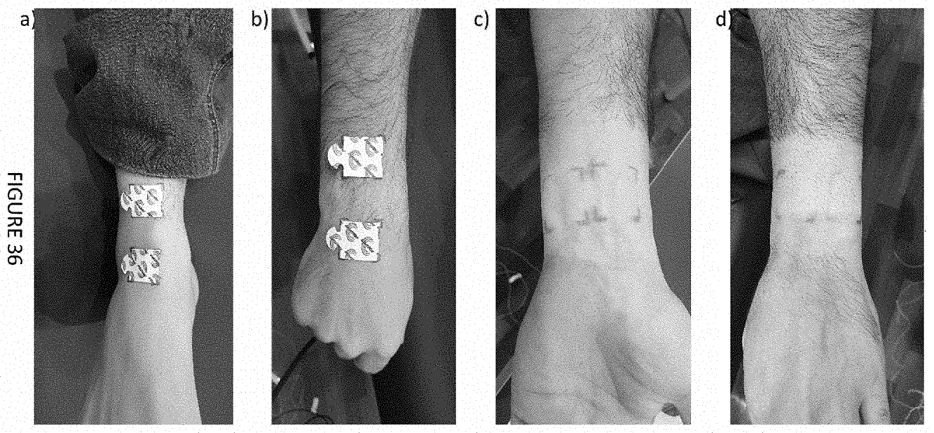

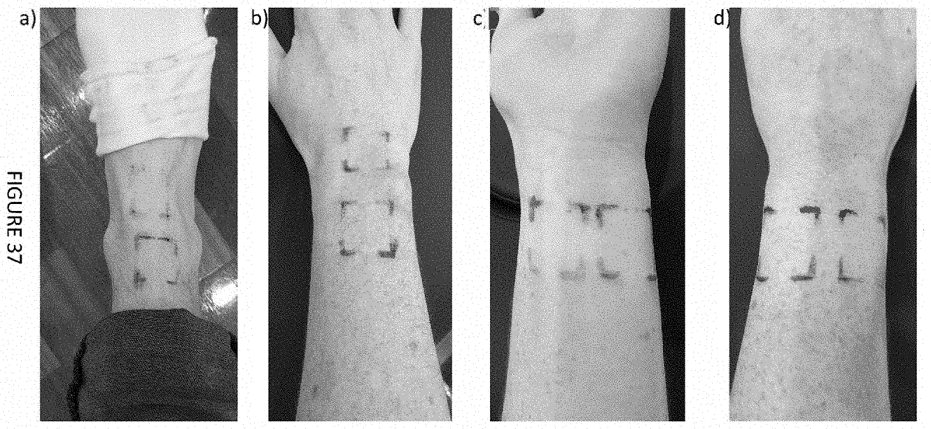

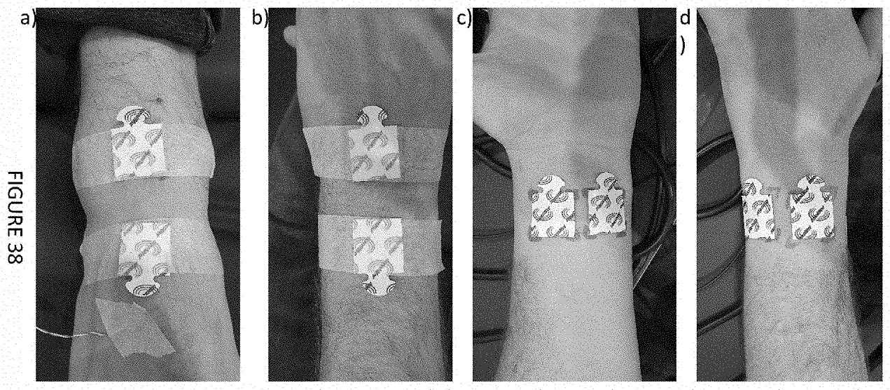

All electrodes were left in place throughout all bioimpedance measurements made within a single run through of the testing protocol. The sites of electrode placement were kept consistent between different run throughs of the testing protocol. The outline of each gel electrode was traced in permanent marker and photos were taken of each site of electrode placement to enable this as shown in FIGS. 36 to 38.

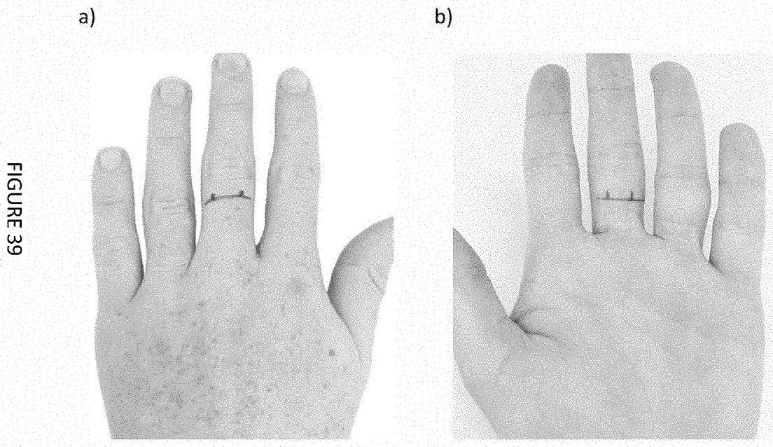

The position of the ring for bioimpedance measurements across the finger was kept consistent by placing it as far down the middle finger as it would go and ensuring the electrodes were making complete contact with the same places on the finger of a participant. Permanent marker was used to guide the placement of the ring and to indicate where the electrodes were placed as shown in FIG. 39.

Before placing the gel electrodes on the wrist and the ring on the finger, the locations of electrode contact were shaved to remove all hair. The sites of electrode placement for full body measurements were not shaved as this had not previously been completed during the human baseline experiments. The gel electrodes were all taped into place with paper tape to ensure they did not move throughout the testing protocol and to maintain proper electrode contact. A small length of tape (approx. 5 cm) was placed through each gel electrode on the hand and foot where full body measurements were taken and a length of tape was wrapped around the entire wrist where gel electrodes were placed for measurements through the wrist.

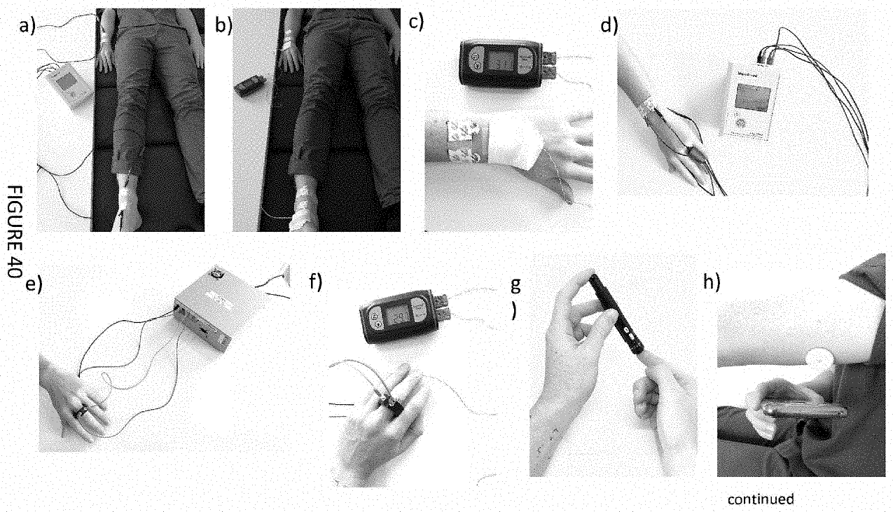

A range of ancillary physiological parameters were measured alongside bioimpedance as these may have influenced the bioimpedance data collected: skin temperature adjacent to the sites of electrode placement, skin pH adjacent to the sites of electrode placement, systolic and diastolic blood pressure, and heart rate as shown in FIG. 40.

For skin temperature and skin pH, where measurements were taken adjacent to the site of electrode placement, the measurements described below were taken as shown in Table 14.

TABLE 14
Skin temperature and skin pH measurements were taken

| Configuration | Skin Temperature | Skin pH |
|---|---|---|
| Full Body | 2 cm below bottom | Between the two |
| | electrode on the feet, | electrodes on the feet |
| | towards the toes | Between the two |
| | 2 cm below bottom | electrodes on the hand |
| | electrode on the hand, | |
| | towards the fingers | |
| Wrist | Immediately below the | Immediately below the |
| | gel electrodes, towards | gel electrodes, towards |
| | the finger; underside | the fingers; upper- |
| | of the arm | side of th earm |
| Finger | Underneath the ring | Immediately below the ring |
| | housing, RHS of the L3 | housing, towards the torso; |
| | finger, towards the thumb | underside of the finger |

Data was recorded from each of the 3 participants during 8 OGTTs (OGTT total=24) over a 5 day testing period. This OGTT procedure involved consumption of a glucose solution (75 g glucose in 300 mL water; from POCD Scientific) and measurement of all parameters at 0, 10, 20, 30, 40, 50, and 60 min timepoints post-consumption.

Measurements were taken for the 0 min timepoint at a recorded time before the drink was consumed. A timer was started immediately after the drink was consumed and each set of measurements were taken after the timer reached 10, 20, 30, 40, 50, and 60 min. The measurements were taken in the following defined order, such that the timepoint at which each measurement of a particular parameter was approximately equal:
1. Skin pH (at 0 min);
   a. Foot;
   b. Hand;
   c. Wrist; and
   d. Finger.
2. Bioimpedance—full body;
3. Skin temperature—full body;
4. Bioimpedance—wrist;
5. Skin temperature—wrist;
6. Bioimpedance—finger;
7. Skin temperature—finger;
8. BGL—Accu-Chek;
9. BGL—FreeStyle Libre;
10. Blood pressure and heart rate; and
11. Skin pH (at 60 min);
   a. Foot;
   b. Hand;
   c. Wrist; and
   d. Finger.

Due to the variable length of time required for taking skin pH measurements, these were only taken at 0 min and 60 min as there was not enough time in the testing procedure to reliably accommodate measurements every 10 min. This meant that the data could not be used as an input in the neural network model.

No caffeine was consumed before or during the testing procedure (unless indicated) and, where possible, the first measurement in a day was recorded on a participant who had been fasting overnight. The data from each measurement was input into a table manually which was then scanned and backed up onto the cloud. For bioimpedance measurements, the data file name was recorded, while the data values were recorded for every other parameter. The finger which was lanced for Accu-Chek measurements was recorded in the appropriate column and any relevant comments were made into the final column. The exact time at which each measurement was made was also recorded into the appropriate field.

Following the testing procedure, data from each OGTT was input into the master Excel document. The ImpediMed data files were uploaded from the device and copied onto the cloud. The data was processed as a batch in the ImpediMed software to generate a spreadsheet with all calculated parameters relevant to each data file included and an additional spreadsheet per data file that included the raw data. The calculated data was copied manually into the master Excel document and the raw data corresponding to each data file (resistance and reactance values at each frequency analysed) were copied into the master Excel document using a macro embedded into this document. The data generated by the EIS instrument was also backed up onto the cloud and the data from these files were likewise copied into the master Excel document using a macro embedded into this document. At the end of each day, the following tasks were completed:
1. ImpediMed data was uploaded onto the cloud;
2. EIS instrument data was uploaded onto the cloud;
3. All data from the printed data sheets was input into Excel and these were scanned and stored;
4. The Accu-Chek and FreeStyle Libre data from each participant were downloaded and backed up;
5. Devices that needed charging were plugged in;
6. pH probe was stored overnight;
7. Testing area was tidied;
8. Rubbish bin was emptied; and
9. Consumables for the following day were prepared.

At the start of the day, the following tasks were completed:
1. ImpediMed was tested using the test cell (required to be passed before the device could be used on a human participant);
2. EIS instrument was tested with a standard resistor (required to be passed before the device could be used on a human participant);
3. ThemaQ devices were tested;
4. Blood pressure meter was tested; and
5. pH probe was calibrated.

Before each test, the following tasks were completed. To speed up the testing process, tasks 2-5 were completed while the testing process was finalised on the prior participant:
1. Dry electrodes were cleaned with isopropanol. This was to ensure there was no residue built up on the electrodes from the previous round of testing.
2. Participant visited the bathroom. This was to empty their bladder which may otherwise affect bioimpedance measurements. Ideally this should have been completed before each full body bioimpedance measurement throughout the testing process, but this was not possible during this experiment due to time constraints.
3. All items were moved from the participants' pockets and any jewelry/metal was removed (e.g. belt with a metal buckle). Objects or metal items may affect the bioimpedance measurements.
4. Participants skin was cleaned with isopropanol wipes at the sites of electrode placement.
5. Gel electrodes were placed and taped in place.
6. Blood pressure meter cuff was fixed. This was placed on the opposite arm of the participant to the one onto which the FreeStyle Libre device was applied.
7. The ring and electrodes were fixed to the finger.
8. Thermocouples were attached. These were taped in place and covered with a folded tissue (also taped in place) to add insulation. The thermocouple placed underneath the ring was not insulated.
9. The EIS instrument was tested for an appropriate response.

The following checklist was used to ensure all tasks had been completed at the start of the day, before each test, during each test, and at the end of the day.

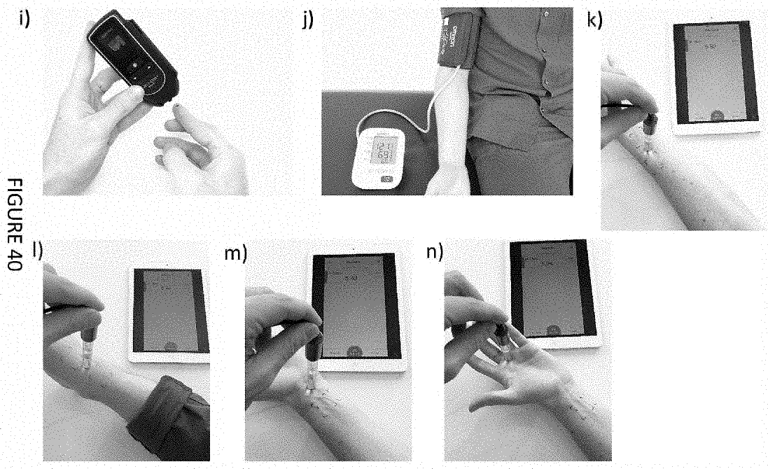

To avoid issues of incorrect lead placement, guides such as FIG. 41 can be provided to illustrate correct lead placement and configuration.

Results

Using OGTTs to manipulate participant BGL, a wider range of BGLs were attained compared to data input into the preliminary predictive model as shown in Table 15. The range, mean, median, and rate of changes of the BGLs varied between participants.

TABLE 15
Range, mean, median, and rate of changes of the BGL varied made
using an Accu-Chek or Freestyle Libre device on participants 1, 2, and 3.

| Blood Glucose | | | |
|---|---|---|---|
| Monitoring Device | Participant 1 | Participant 2 | Participant 3 |
| Accu-Check ® | RANGE (mmol/L) | RANGE (mmol/L) | RANGE (mmol/L) |
| | 5.7 (4.4-10.1) | 3.5 (4.2-7.7) | 7.7 (5.0-12.7) |
| | MEAN (mmol/L) | MEAN (mmol/L) | MEAN (mmol/L) |
| | 7.3 | 5.8 | 8.1 |
| | MEDIAN (mmol/L) | MEDIAN (mmol/L) | MEDIAN (mmol/L) |
| | 7.3 | 5.9 | 7.9 |
| | RATE OF CHANGE | RATE OF CHANGE | RATE OFCHANGE |
| | ((mmol/L)/min) | ((mmol/L)/min) | ((mmol/L)/min) |
| | −0.17-0.23 | −0.17-0.22 | −0.27-0.40 |
| Freestyle Libre | RANGE (mmol/L) | RANGE (mmol/L) | RANGE (mmol/L) |
| | 5.2 | 3.5 | 5.5 |
| | MEAN (mmol/L) | MEAN (mmol/L) | MEAN (mmol/L) |
| | 6.7 | 6.1 | 7.2 |
| | MEDIAN (mmol/L) | MEDIAN (mmol/L) | MEDIAN (mmol/L) |
| | 6.7 | 6.2 | 7.1 |
| | RATE OF CHANGE | RATE OF CHANGE | RATE OF CHANGE |
| | ((mmol/L)/min) | ((mmol/L)/min) | ((mmol/L)/min) |
| | −0.17-0.19 | −0.16-0.28 | −0.11-0.16 |

Once all the data had been collected for this dataset, the average values and range for each parameter were determined to inform the range over which these parameters should be expected in future experiments. These were calculated and shown for BGL (Accu-Chek, FreeStyle Libre, OGTT), skin temperature (hand, foot, wrist, finger), skin pH (hand, foot, wrist, finger), blood pressure, heart rate, body water content, and fat percentage.

### EXAMPLE 9

### Predictive Model

Intent

As discussed above, it was found that it was possible to use predictive models to correlate bioimpedance measurements (BI) with blood glucose concentration in study participants. The Artificial Neural Network modeling approach used in this study was continued and modified due to their ability to identify hidden correlations, as well as having sufficiently flexible architecture and parameters for continuous model improvement.

The minimum requirement for the model success criteria continued being the model's ability to predict BGL such that at least 70% of results were in zones A and B based on Parkes Error Grid (PEG) zones.

This modeling work is based on impedance measurements obtained from both an ImpediMed® device and an EIS instrument across the full body, wrist, and finger configurations. For consistency, the Accu-Chek® SMBG meter was used to obtain pin-prick BGL readings to serve as reference values for training and testing the model. This modeling work used data obtained during human trials and has been used to inform subsequent model-development decisions.

Source Data

Bioimpedance data obtained for the Full Body and Wrist configurations from the ImpediMed® device includes raw values and processed fitted-parameters determined by the device. Data obtained from the ImpediMed® device, included:
Raw data of Reactance and Resistance at 256 frequencies:
   Frequency (kHz), also referred-to as F in the raw data;
   Resistance (Ohms), also referred-to as R in the raw data; and
   Reactance (Ohms), also referred-to as X in the raw data.
Device-processed data resulting in fitted parameters:
   Cole fit centre X, ohms;
   Cole fit centre R, ohms;
   Cole circle radius, ohms;
   SEE of radius, % R (zero), ohms;
   R (infinity), ohms;
   Re, ohms;
   Ri, ohms;
   Z characteristic, ohms;
   f characteristic, kHz; and
   Membrane capacitance, nF.

From the EIS instrument, the following information was used for modeling for the Finger configuration:
Z (impedance), ohms;
Phase (angle), degrees;
G (conductance), Siemens; and
C (capacitance), Farads.

In addition to bioimpedance data, the following ancillary data was also recorded during the testing:
Participant height;
Participant weight;
Participant age;
Participant gender; and
Skin surface temperature.

The data was collected over a period of 3 days. There were 42 samples each for participants 1 and 2, and 28 samples for participant 3. For every BGL data point obtained from Full Body and Wrist, there were 5 impedance results associated with that value due to repetitions during data collection. There were 3 impedance results per BGL value.

Data Scaling

The dataset can be numerically scaled to work well with the neural network package due to the algorithms used. Two potential methods were explored for scaling:

Normalisation: rescaling the values into a range of 0 to 1, inclusive. This approach is typically useful when parameters need to have the same positive scale, however outliers from the data set would be lost; and

Standardisation: rescaling the data to have a mean of 0 and standard deviation of 1 around the mean.

Standardisation was selected to avoid discarding outliers automatically as it was deemed necessary to visualise and make manual decisions judging from the spread of data. To this end, the “dataPreparation” package in R was selected.

Within this package, the “build_scales” function was used to compute the scale to be used based on the Training dataset. From that point, the “fastScale” function was used to scale the Training dataset and later the Testing dataset based on the former.

Model Information

As with the preliminary model, a two-layer neural network model with 3 nodes per layer was used in the present neural network model. A linear activation function was used. The full data set was split approximately 80:20 between training and testing sets for each participant, and the data points were selected at random.

Randomness within the model was seeded to ensure reproducibility of results between test runs and also for different computers the testing was conducted on.

A 5-fold cross-validation was conducted where the model was trained and validated five times. For each round of training, the training set was randomly split 60-40 between training and validation. That is 60% of the data used for training, 40% for validation, with a different set of data for each round of training.

Model performance was assessed via calculation of the Mean Squared Error (MSE), Mean Absolute Relative Difference (MARD), and proportion of predicted points within the Parkes Error Grid (PEG) Zones.

Model Implementation

Modeling results are shown in Table 16.

TABLE 16
Results from neural network modelling for data from Study
Participants 1, 2, and 3, for full body, wrist and finger.

| | | Training + | Training + | Training + |
|---|---|---|---|---|
| | | Validation | Validation | Validation |
| Study Area | Dataset Used | MSE | MARD | PEG A (%) |
| Full Body | Cole Fitted Parameters (Cole fit | 0.67 | 9.69 | 93.1 |
| | centre X, ohms Cole fit centre R, | | | |
| | ohms Cole circle radius, ohms | | | |
| | SEE of radius, % R (zero), ohms | | | |
| | R (infinity), ohms Re, ohms Ri, | | | |
| | ohms Z characteristic, ohms f | | | |
| | characteristic, kHz Membrane | | | |
| | capacitance, nF) | | | |
| Full Body | Cole Fitted Parameters with | 0.26 | 6.00 | 100.0 |
| | Additional Metrics (Temperature | | | |
| | (Upper Body) (° C.), Skin | | | |
| | Temperature (Lower Body) (° C.), | | | |
| | Systolic Blood Pressure, | | | |
| | Diastolic Blood Pressure, Heart | | | |
| | Rate) | | | |
| Wrist | Cole Fitted Parameters (Cole fit | 0.71 | 9.87 | 93.1 |
| | centre X, ohms Cole fit centre R, | | | |
| | ohms Cole circle radius, ohms | | | |
| | SEE of radius, % R (zero), ohms | | | |
| | R (infinity), ohms Re, ohms Ri, | | | |
| | ohms Z characteristic, ohms f | | | |
| | characteristic, kHz Membrane | | | |
| | capacitance, nF) | | | |
| Wrist | Cole Fitted Parameters with | 0.53 | 7.99 | 93.3 |
| | Additional Metrics (Temperature | | | |
| | (Upper Body) (° C.), Skin | | | |
| | Temperature (Lower Body) (° C.), | | | |
| | Systolic Blood Pressure, | | | |
| | Diastolic Blood Pressure, Heart | | | |
| | Rate) | | | |
| Finger | EIS Instrument Ouputs | 0.54 | 7.36 | 93.9 |
| | (Z[Ohms], Phase[Deg], G[S], | | | |
| | C[F]). | | | |
| Finger | EIS Instrument Ouputs with | 0.42 | 6.92 | 94.3 |
| | Additional Metrics (Temperature | | | |
| | (° C.), Systolic Blood Pressure, | | | |
| | Diastolic Blood Pressure, Heart | | | |
| | Rate) | | | |

| Training + | Training + | | | | | |
|---|---|---|---|---|---|---|
| Validation | Validation | Testing | Testing | Testing | Testing | Testing |
| PEG B (%) | PEG C (%) | MSE | MARD | PEG A (%) | PEG B (%) | PEG C (%) |
| 6.9 | 0.0 | 1.21 | 11.69 | 84.5 | 15.5 | 0.0 |
| 0.0 | 0.0 | 2.71 | 19.83 | 100.0 | 0.0 | 0.0 |
| 6.9 | 0.0 | 2.76 | 15.39 | 69.1 | 30.9 | 0.0 |
| 6.7 | 0.0 | 1.43 | 13.58 | 79.1 | 20.9 | 0.0 |
| 5.7 | 0.4 | 2.96 | 21.52 | 52.8 | 47.2 | 0.0 |
| 5.7 | 0.0 | 3.88 | 24.64 | 44.4 | 51.4 | 4.2 |

Overall for the case of data from the ImpediMed®, the model is able to use bioimpedance (fitted parameters) to predict blood glucose concentration when attempting to model with measurements from the full body and wrist: 100% of PEG Points were located in the A+B zones, with the inclusion of ancillary physiological parameter measurements beyond BI (e.g. Temperature, Heart Rate) improving prediction capabilities.

Further to this, for the case of data from the EIS instrument for the finger, the model is also able to use raw bioimpedance values to predict blood glucose concentrations: almost 100% of PEG Points were located in the A+B zones, with the inclusion of ancillary measurements beyond BI (e.g. Temperature, Heart Rate) improving prediction capabilities for the Training dataset but not the Testing dataset.

Conclusions

Overall, the concept of using bioimpedance (BI) to predict blood glucose concentration within the parameters of this study and data collected continues to be workable for the full body, wrist and finger configurations using data from the ImpediMed® and the EIS instrument.

It can be concluded that the neural network model constructed is fit for the intended purpose, exceeding the study success criteria of 70% of predicted points within the A and B zones on a Parkes Error Grid.

### EXAMPLE 10

### Pre-Market Pilot/Early Feasibility Study

Intent

After collecting BGL data from participant 1 and participant 2 as part of the human baseline measurements, it was noted that only a limited range of BGL values were achieved (4.1-8.9 mmol/L for participant 1 and 4.4-6.5 mmol/L for participant 2, as per Accu-Chek), all of which were in the range of people who did not have diabetes.

Although modifications were made to the testing procedure to broaden the range of BGL values achieved prior to human testing, the ideal pathway would have been to recruit people with poorly managed diabetes that would be expected to produce a far wider range of BGL values.

Exploratory investigations were undertaken about the possibility of conducting a pre-market pilot/early feasibility study in this current phase of the study, considering the timeframe to complete all requirements.

Background—Clinical Trials of Medical Devices

Clinical trials of medical devices proceed through “stages” rather than “phases”, which include:

Pre-market pilot: 10-30 participants. Exploratory investigations to gather preliminary clinical safety and performance information to guide device modifications or provide support for a future pivotal study. Conducted following other non-clinical testing (engineering analysis and testing, computational simulation, biocompatibility testing, and, where appropriate, animal testing). Includes first in human and feasibility or proof of concept studies.

Pre-market pivotal: 100+ participants. Confirmatory investigations to evaluate device performance and safety for a specified intended use to satisfy pre-market regulatory requirements.

Post-market: 1000+ participants. Confirmatory investigations to establish performance and safety in broader populations or observational investigations to gain a better understanding of device safety, long-term outcomes, and health economics.

Experiment Details

The details of the pre-market pilot/early feasibility study that were investigated as a possibility during the current phase of the study are below:

Purpose: to achieve a wider range of BGL recordings by recording data from participants with diabetes to strengthen the neural network model correlating BGL and bioimpedance.

Participants: 10 adults aged between 18-50 years who have been diagnosed with type 2 diabetes in the last 10 years. Preference for participants with suboptimal oral management as indicated by a HbA1c range between 7-8.5%.

Experiment: Simultaneously record participant BGL (using an Accu-Chek Mobile device) and bioimpedance every 15 min over a 5 h period during which participants are non-fasting, consume a meal, and are using their usual BGL manipulation regime. A similar procedure is used in: Staal, O. M., et al. (2018). Biosensors 8(4): 93.

Considerations: (1) how to recruit participants, (2) whether our study is considered a clinical trial, and (3) whether ethics approval is required.

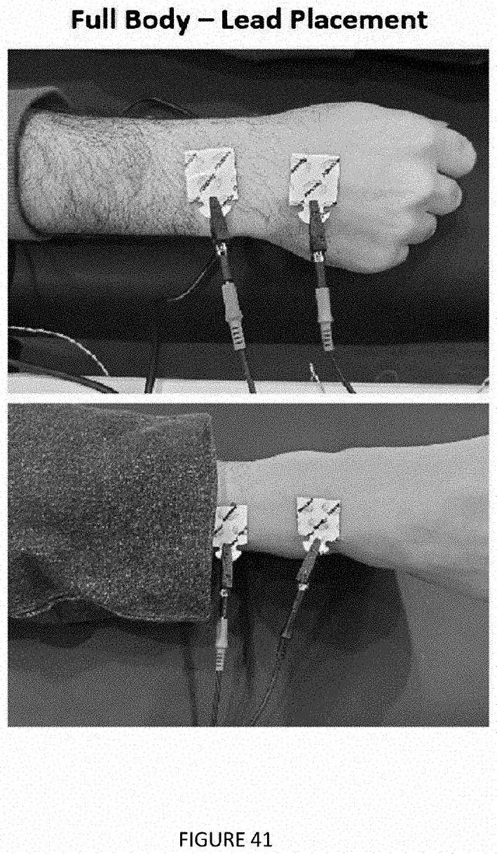

A summary of the potential devices that were to be used and the required clinical trial/ethics considerations are shown in FIG. 42. The middle-dashed option is the one that was relevant at this stage. The left-dashed option is the one that was possible to do however it did not provide any further value to the study at the current stage.

### EXAMPLE 11

### Additional Developments

Intent

The non-invasive device of the present invention can be further developed, for example, optimising device sensitivity and accuracy, and studying the use of the wearable in more ‘real world’ situations.

EIS Instrument

Signal Leakage

When measuring bioimpedance using the device's electrodes, the generated stimuli signal appeared to have a leakage path. The signal leakage did not affect the quality of the measurement. The generated stimuli signal is an important indicator in showing how much of the stimuli signal that had been applied through the portion of the body was being measured by the voltage sensing electrodes.

This can only be determined once it has been ensured that the internal electronics of the EIS instrument do not provide any path for leakage and that the signal is instead leaking through a path across the body where the wearable's voltage sensing electrodes are not detecting it. The implications around this issue could be (i) inefficient electrode design causing a large portion of the applied signal being unmeasurable; and (ii) potential device safety compliance issues if the applied signal levels have to be increased to overcome the design inefficiency.

Wearable Ring

Although acceptable performance of the EIS instrument was achieved using a ring design for the non-invasive wearable device, optimisation of sensitivity and accuracy can still be performed.

Electrode Configuration

A single ring configuration was tested with the EIS instrument before moving forward with human testing experiments.

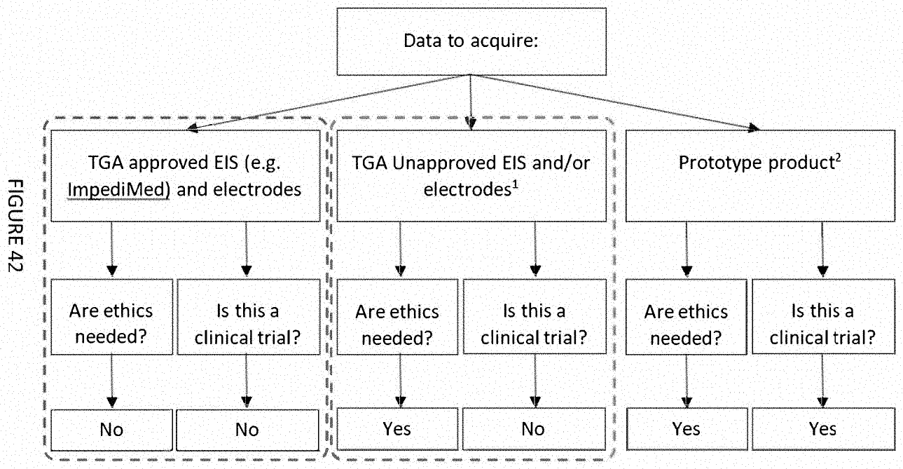

Without being bound by anyone theory, the present Applicant believes the ideal configuration will be a 4-terminal configuration with the position of the voltage sensing electrodes being in the middle of a uniformly distributed current field. FIG. 43 illustrates an alternative embodiment of the electrode configuration of the ring.

Electrode Surface Area

Electrodes with smaller surface areas can be developed. In one embodiment of the ring current configuration, large capacitances are seen at low frequencies. Reducing the surface area of the voltage sensing electrodes may be address this issue and can improve sensitivity.

Wearable Watch

Conventional watch design for the wearable devices differs from a bracelet in that a watch only measures bioimpedance across one side of the wrist whereas a bracelet measures bioimpedance through the portion of the body (being the wrist). However, a wearable watch can be developed to measure bioimpedance through the portion of the body (such as the wrist).

Interferences

External Interferences

During system testing, impact of external electrical interferences can occur affecting the quality of the EIS instrument measurements. A Faraday Cage can be used to shield the EIS instrument from these interferences, generating high quality and repeatable measurements.

Although a Faraday Cage can be used to maintain signal quality in a controlled lab environment, it may not be practical for the non-invasive device depending on the configuration. However, an optimised Faraday cage could be developed to provide a portable non-invasive wearable device.

Internal Interferences

Another type of interference affecting the quality of bioimpedance measurements was generated from movement of the subject who had the device fitted.

Bioimpedance Signal Quality

Baseline readings and “smart” troubleshooting

The wearable can detect baseline readings and provide basic troubleshooting to the user wherever possible. An abnormally low bioimpedance measurement, for example, may mean that the electrodes have been shorted by moisture or water on the skin. An abnormally high bioimpedance, conversely, may indicate poor electrode contact.

The non-invasive device of the present invention can be “smart” such that it can distinguish between poor quality measurements and for it to provide informative warnings to the subject instead of providing erroneous blood glucose concentration readings.

Stress Testing

All experiments in this study were highly controlled and bioimpedance measurements were made in consistent positions and conditions (e.g. participant lying down, limited movement, no electrical devices nearby, and the wearable and EIS instrument in the exact same position).

These highly controlled experiments do not represent the ‘real world’ use of a wearable device. A wearable device will be exposed to consistent movement, uncontrolled changes in homeostasis (e.g. hydration level, sweating), and external influences such as washing, cream application (e.g. sunscreen, moisturiser), and hair growth.

Each of these influences must be explored to determine their effect on bioimpedance measurements. For the existing ring or bracelet wearables, the influences of body position (laying down, standing up with arm in the air, standing up with arm pointing down), activity (before, during, and after), and hydration (before drinking, after drinking, full bladder, drained bladder), for example, can be further studied.

Wearable Position

The position on which each electrode is position can be optimised. The ring embodiment, for example, has only been investigated in a single position on a single finger (L3) of each participant in the Examples. Different orientations and use on different fingers can be used.

A non-exhaustive listing of some of the novel and/or inventive features of the present invention comprises:

a) Using bio-impedance to measure the blood glucose on a finger continuously and non-invasively;

b) Using bio-impedance in combination with other biometrics (including body temperature, pH, blood pressure) to measure the blood glucose on a finger continuously and non-invasively;

c) Using bio-impedance to measure the blood glucose on a human body part continuously and non-invasively;

d) Using bio-impedance in combination with other biometrics (including body temperature, pH, blood pressure) to measure the blood glucose on a human body part continuously and non-invasively;

e) Using artificial neural network (ANN) model to correlate the measured biometrics (including but not limited to bioimpedance, body temperature, pH, blood pressure) to blood glucose;

f) Using different ANN architecture for different form factors (whether it is a ring or a bracelet or in other form);

g) Using a dynamic adaptive ANN, which enables the ring to adapt to the specific biometrics patterns of the user, and thus increased accuracy as the user keeps wearing it;

h) Using a wide range of frequency, from 0.1 Hz to 1 MHz, to measure the bio-impedance;

i) Using high-quality signals to feed the ANN model: the measurement method enables checking of the quality of the output electrical current signals before using it. As a result, filtering of the noisy and low-quality signals could be achieved and only the high-quality ones for the ANN could be used to increase the model's accuracy or enable model's functionality;

j) Positioning of the electrodes in the ring: The existing devices place electrodes only on one side of the body part (i.e. wrist), whereas in our proposed device (i.e. ring) electrodes are placed in a configuration to allow electrical current passing through the body part rather than just through the skin;

k) Adjustable electrode contact mechanism to ensure receiving high-quality signals while maintaining comfort: The contact areas of electrodes are automatically being adjusted to ensure there is a proper contact between the electrode and skin to receive high-quality signals;

l) Adjustable electrode configurations to ensure receiving high-quality signals. That is position of current source and sink, and voltage sensing electrodes can be changes in PCB (not physically) to ensure receiving high-quality signals; and

m) Electrodes can be fitted in gadgets or come in form of patches suitable for use in mobile electronic devices (such as mobile phones, iPad, iPod, etc.).

### EXAMPLE 12

### Alternative Device Configurations

Optimising Signal Quality

Different parameters of a non-invasive device of the present invention can be adjusted to optimise bioimpedance data depending on the desired wearable device and configuration. The attributes of high signal quality, low data variability, and a low magnitude of bioimpedance are preferred for a non-invasive device of the present invention such that the device can be sensitive to biological systems. The electrode design system can be sensitive to biological systems to identify variation in biological parameters, for example blood glucose level.

Bioimpedance signal quality: Signal quality was qualified by measuring the noise and distortion levels of the bioimpedance signal. These parameters were evaluated from the bioimpedance sensor's raw waveform output and the Discrete Fourier Transform Quality of Fit (DFT QOF) output.

Magnitude of bioimpedance: Any electrode system can make significant contributions to the magnitude of bioimpedance of the sample being analysed. Minimising this contribution is recommended to maximise the relative contributions of changes in the biological (e.g. changes in BGL). This enables overall greater sensitivity. The magnitude of bioimpedance was examined across the full frequency range.
Repeatability: Ensuring repeatability when measuring a single sample under stable conditions is preferable to minimise the error potentially introduced into bioimpedance measurements which would otherwise affect the workability of a predictive BGL model. Examining deviations in the bioimpedance measurements when examining single samples under stable conditions provides a measure of repeatability.

Electrode Arrangement and Spatial Positioning

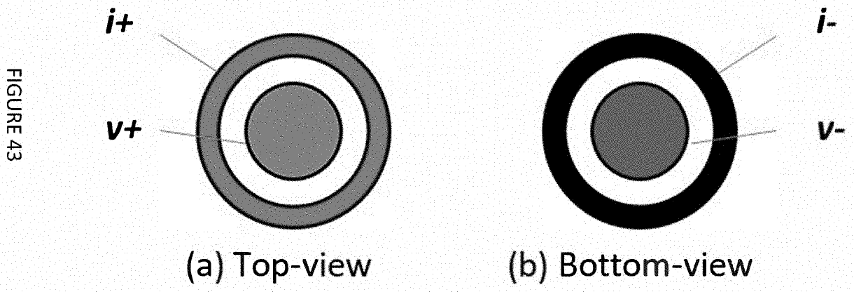

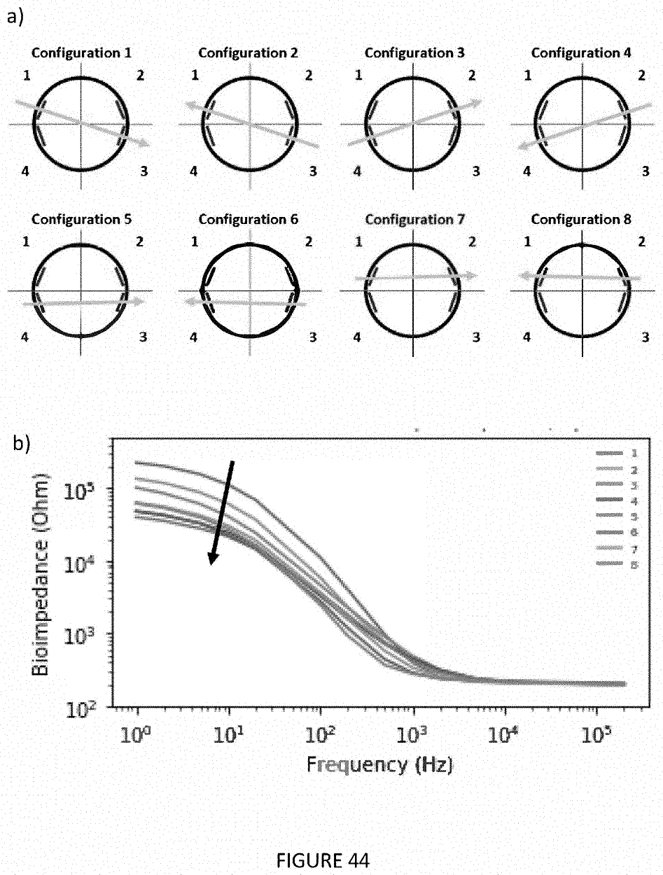

A non-invasive wearable device in the form of a four-electrode ring was evaluated to compare bioimpedance results generated with sensing electrodes placed on the same or opposite side of the current path as shown in FIG. 44. The arrows indicate the flow of current passing through a portion of the body between the current injecting electrodes. The remaining two electrodes are voltage measurement electrodes. The two current injecting electrodes are configured to be substantially opposed and the two voltage measurement electrodes are configured to be radially spaced between about greater than about 30° to about 60° relative to each of the current injecting electrodes and are substantially opposed to each other. An example of the four-electrode ring device is shown in FIG. 45a and an exemplary non-invasive device is shown in FIG. 45b.

Bioimpedance data was recorded using an EIS instrument using the 8 different configurations as shown in FIG. 44. These were intended to evaluate both the position of the sensing electrodes and the direction of current flow. A representative bioimpedance result for the 8 different configurations is shown in FIG. 44. The configuration with sensing electrodes on the opposite sides of the current path are configurations 1-4 and sensing electrodes on the same side of the current path are configurations 5-8. Greater variability and higher bioimpedance was observed using configurations 1-4, while lower variability and lower bioimpedance were seen using configurations 5-8. These configurations demonstrate the arrangement of the i−, i+, v−, and v+ electrodes (i.e. fixed electrode positions, but changing which electrode is i−, i+, v−, and v+)

Lower bioimpedance was observed with sensing electrodes on the same side of the current path (configurations 5-8). The spatial position of these electrodes can be important as the position can determine how current flows through the user's finger while wearing the ring. Optimal spatial electrode position was determined by testing eight different current and voltage positions on the ring as shown in FIG. 44. Positions were chosen as they are all possible permutations and combinations for current and voltage electrodes in a four-electrode system. Positions were assessed on the magnitude of bioimpedance, and quality of waveforms generated.

A four electrode device was then divided into 2 current injecting (i−, i+) and 2 voltage measurement (v−, v+) electrodes (configurations 5-8).

Bioimpedance was then measured using an EIS machine on one participant. It was consistently shown that current injecting electrodes (i− and i+) on the same side and sensing electrodes (v−, v+) opposite the current electrodes produce consistent and reliable bioimpedance data (configurations 5-8). Across three repetitions, the data had a lower magnitude of impedance, favourable waveform data and ‘gold standard’ type phase angle data. The present inventors surprisingly found that placing current injecting and voltage measurement electrodes opposite each other (configurations 5-8) produced better signal quality; making the device more suited to detecting changes in a biological system.

Another factor to be considered is the spacing and/or spatial positioning between the electrodes. This is because, the actual physical placement of the electrodes and distance between can determine which part of the finger makes contact with the ring. Preferably, electrodes should be spaced far enough apart to reduce the risk of a short circuit in the device and also be sufficiently far enough apart to ensure the current passes through a large flow path of a subject to maximise the amount of tissue over bone.

This was tested by having a participant taking bioimpedance measurements using an EIS machine with a voltage measurement electrode that was spaced 30° apart and 60° apart from the current injecting electrode in a four electrode device as shown in FIG. 45. The current flow path was horizontal through the finger, that is the plane parallel to the plane of the palm of a hand.

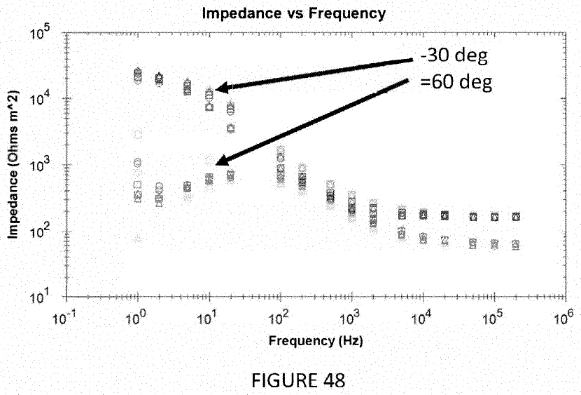

FIG. 48 shows the effect of electrode spacing on bioimpedance measurements. The 30° ring device had higher impedance but less variation between repetitions than the 60° ring device. It was also observed that the signal amplitude for the 30° ring device was higher than the 60° ring device. The 60° ring device had a very low signal amplitude causing high variability in bioimpedance measurements. The 30° ring device had a higher signal amplitude than the 60° ring device and also had higher impedance which lowered sensitivity to bioimpedance changes. The ring device with 30° electrode spacing was preferred.

These relative angles were chosen as any angle smaller could potentially increase the probability of a short circuit, and too far apart would can potentially be uncomfortable. However, as would be appreciated by a skilled addressee, angles less than 30° and angles greater than 60° can still be used in the present invention. The present inventors surprisingly found that electrodes at a 30° angle had a better signal quality and lower impedance compared to electrodes spaced at a 60° angle. The 30° embodiment also had lower magnitude of bioimpedance, which increased its sensitivity to bioimpedance changes.

It was also found that if the ring device is maintained at a 30° angle, if the size of the ring is decreased, the likelihood of a short circuit can increase. The present inventors surprisingly found that a ˜1 mm gap between two electrodes can provide more flexibility to ring device design as the gap can be used for all ring sizes. This can provide the angle between two electrodes to change while maintaining a suitable distance between the electrodes as the ring size varies.

The following calculations also can provide an indication that the angle remains between 24° to 33° for rings with a size of 17-24 mm (see below).

θ=angle between electrodes

θ=(1.5 mm×360°)/2πr for a ring size=17-24 mm

r=(ring size)/2πr

Therefore, angle of 33°-24° between two electrodes.

Electrode Shape

Different electrode shapes were used to compare the bioimpedance results generated. Electrodes were either square or circular-shaped. Bioimpedance data was recorded using an EIS instrument using square (5×5 mm diameter, 25 mm²) or circular (5 mm diameter, 19.63 mm² surface area) electrodes. Data was acquired in sequence across 3 positions and 3 replicates on 1 participant. Two current flow path configurations (configurations 1 and 5) were tested.

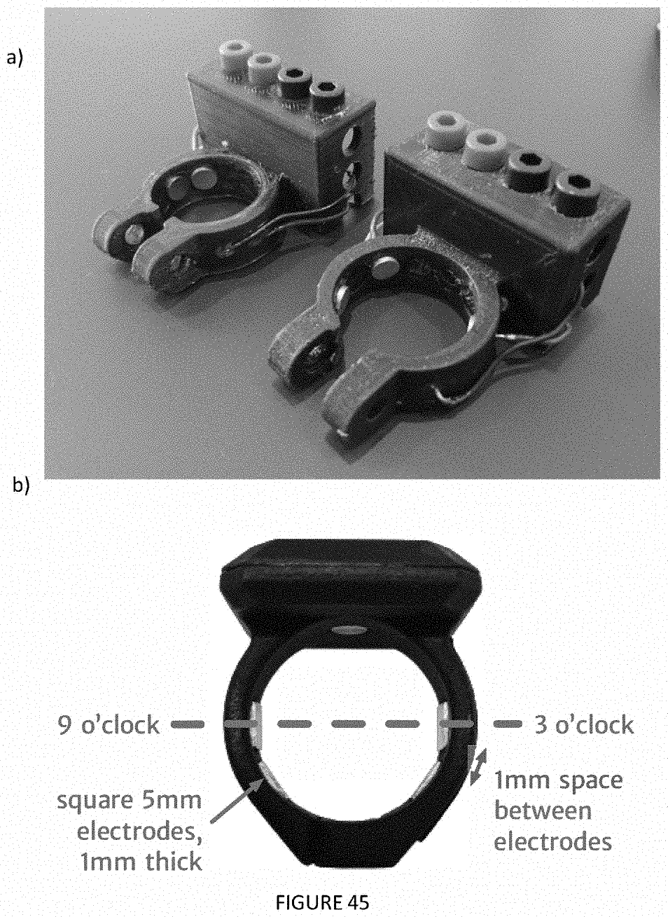

A representative bioimpedance result for a square and circular electrode is shown in FIG. 46. A substantially lower magnitude of bioimpedance was observed with a square electrode. A typical, “stepped” shaped plot was observed with a square electrode demonstrating higher sensitivity of the wearable device to the layers of a finger.

The present inventors surprisingly found that square electrodes reliably decreased the magnitude of impedance and produced much lower variability compared the circular electrodes. The Applicant believes that this is the first time that it has been shown that square electrodes produce better results compared to circular electrodes and demonstrates that square electrodes can be more sensitive in detecting changes in biological parameters associated with blood glucose levels.

Electrode Size/Surface Area

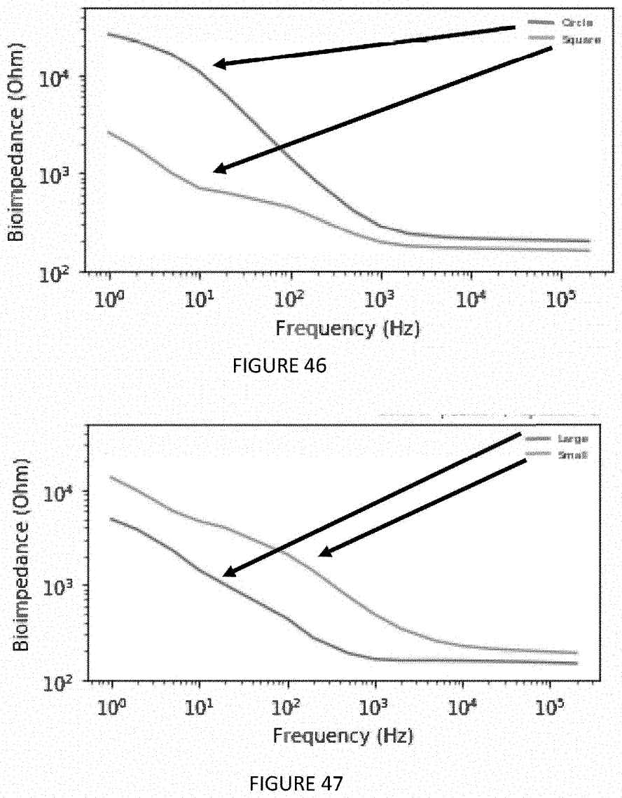

Different electrode sizes/surface areas were used to compare the bioimpedance results generated. Bioimpedance data was recorded using an EIS instrument using 2 different sizes using configuration 5 where the voltage measurement electrode was adjusted between the two devices (5 mm×5 mm square electrode ‘large’ and 2.5 mm×2.5 mm ‘small’ square electrode) but the current injecting electrodes were the same size (5 mm×5 mm square electrode). Data was acquired in sequence across 3 positions and 3 replicates on 2 participants. A representative bioimpedance result using electrodes of different sizes is shown in FIG. 47.

As would be appreciated by a skilled addressee, the size and therefore surface area of the electrodes include the voltage measurement electrodes can affect the contact area between the skin of a subject and electrode. This can influence the signal quality, since a greater size increases the contact area, and hence electrical contact, with the skin.

Despite good waveforms for both the large and small square electrode rings, it was observed that the large voltage electrodes reduced the magnitude of bioimpedance and hence the sensitivity in detecting blood glucose level changes was increased. The large voltage measurement electrodes of 5 mm by 5 mm were preferred in the design of the ring in one embodiment for their increased sensitivity over smaller electrodes. The present inventors believe that prior to the subject invention, the specific size for electrodes used for optimising bioimpedance measurements on a non-invasive device had not been identified.

Smaller electrodes (down to 1 mm) had higher contributions to the bioimpedance magnitude. While increasing the size of electrodes (up to 8 mm or even greater) may have further reduced the magnitude of bioimpedance, this was balanced against material cost and design constraints fitting into a ring. The present inventors surprisingly found that by having the same size current injecting and voltage measurement electrodes provided optimal results.

Ring Position

Different positions of the ring device in use and different electrode configurations (configurations 1, 2, 5 and 6) were used to compare the bioimpedance results generated. It was found that a horizontal current flow path on a finger consistently had a lower magnitude of impedance than a vertical current flow path on a finger. A horizontal placement was preferred. A horizontal current flow path is current flow which runs along the plane parallel to the plane of the palm of a hand and a vertical current flow path is in a plane perpendicular to the plane of the palm of a hand. Without being bound by any one theory, the present inventors believe that when the ring is horizontal the voltage and sensing electrodes have minimal interference with the signal from the bone as the outer parts of fingers are denser with tissue. In contrast, when the ring is vertical, the electrical signals the device produced has to bypass more of the bone in the finger producing a higher impedance. It was found that changing the position of the positive or negative current or voltage sensing electrodes had no effect on the magnitude of impedance.

Ring Tightness/Contact Pressure

Different tightness of the ring device on a subject was used to compare the bioimpedance results generated. Tightness, as a measure of contact pressure, is important in ensuring that the electrodes in the ring device makes adequate contact with the skin of a subject. Bioimpedance data was recorded using an EIS instrument from each of 3 rings with 1 mm size differences. The rings were either fit tight (19 mm), measured fit (20 mm), or loose fit (21 mm). These sizes were chosen as they are ±1 mm from the measured diameter of the participant's middle finger. Data was acquired in sequence across 3 positions and 3 replicates on 1 participant. It was found that the 19 mm and 20 mm ring show typical variability of bioimpedance data after removing and replacing a ring device, while the 21 mm ring device which was loose shows significant variability.

Across all measurements, the tight and measured fit rings produced consistent results (low impedance profile, quality phase angle, reproducible signal quality). It was found that an increase the contact pressure did not result in a change in the quality of bioimpedance data generated, but rather a trade-off with comfort. Poor quality bioimpedance measurements were seen when contact pressure was reduced and comfort was increased with the loose fit ring.

Contact pressure should be tailored for each user, such that it is comfortable enough to generate ideal bioimpedance data that it is sensitive to biological systems.

Appropriate fit can be achieved using a device/ring sizing kit to match a participant's fingers to their most appropriate ring size.

Those skilled in the art will appreciate that the invention described herein is susceptible to variations and modifications other than those specifically described. It is understood that the invention includes all such variations and modifications which fall within the spirit and scope of the present invention.

1-52. (canceled)

53. A non-invasive device for determining blood glucose concentration in a subject, the device comprising:
at least two electrodes for contacting the subject's skin and adapted to be connected to a receiver for measuring an impedance signal; and
a housing adapted to receive the electrodes;
wherein the electrodes are configured such that an electrical current passes through a portion of a subject in use.

54. The non-invasive device according to claim 53, further comprising a probe.

55. The non-invasive device according to claim 53, wherein a single electrode can inject current and measure voltage.

56. The non-invasive device according to claim 53, wherein the electrodes independently inject current and measure voltage.

57. The non-invasive device according to claim 53, wherein the device comprises four electrodes.

58. The non-invasive device according to claim 57, wherein two electrodes inject current and two electrodes measure voltage.

59. The non-invasive device according to claim 53, wherein the electrodes are configured to be radially spaced between about greater than about 20° to less than about 180° about a point of reference.

60. The non-invasive device according to claim 53, wherein two electrodes are substantially opposed to each other.

61. The non-invasive device according to claim 60, further comprising two additional electrodes configured to be radially spaced between about greater than about 5° to less than about 80° relative to the two electrodes.

62. The non-invasive device according to claim 53, wherein the electrodes are substantially square shaped.

63. The non-invasive device according to claim 57, wherein the voltage measurement electrode is spaced to provide a gap of between about 0.2 mm to about 1 cm relative to a current injecting electrode.

64. The non-invasive device according to claim 53, wherein the electrode comprises a coating

65. The non-invasive device according to claim 53, wherein the surface area of an electrode is between about 2 to 100 mm².

66. The non-invasive device according to claim 53, comprising an adjustable electrode contact mechanism.

67. A method for non-invasively determining blood glucose concentration in a subject, the method comprising the steps of:
measuring impedance through a portion of the subject using at least two electrodes in conductive contact with the subject's skin; and
determining the amount of blood glucose in the subject based upon the measured impedance,
wherein the at least two electrodes are in a configuration which passes electrical current through the portion of the subject.

68. The method of claim 67, further comprising measurement of at least one additional physiological parameter of a subject.

69. The method of claim 67, wherein the impedance is measured at a plurality of frequencies.

70. The method of claim 67, wherein the measurement is performed at a frequency range of between about 0.1 Hz to about 1 MHz.

71. The method of claim 67, further comprising use of an artificial neural network.

72. A kit comprising:
at least two electrodes adapted to be connected to a receiver for measuring an impedance signal; and
a housing adapted to receive the electrodes.
